(19) United States
(12) Patent Application Publication
(10) Pub. No.: US 2025/0368713 A1
(43) Pub. Date: Dec. 4, 2025

(54) COMPOSITIONS, SYSTEMS AND METHODS FOR MANIPULATING AREA POSTREMA (AP) NEURONS BASED ON GFRAL SENSING

(71) Applicants: Duke University, Durham, NC (US); Cold Spring Harbor Laboratory, Cold Spring Harbor, NY (US)

(72) Inventors: Z. Josh Huang, Durham, NC (US); Yongjun Qian, Durham, NC (US); Bo Li, Cold Spring Harbor, NY (US); Qingtao Sun, Cold Spring Harbor, NY (US); Daniëlle van de Lisdonk, Cold Spring Harbor, NY (US)

(21) Appl. No.: 19/241,135

(22) Filed: Jun. 17, 2025

Related U.S. Application Data
(63) Continuation of application No. PCT/US23/84968, filed on Dec. 19, 2023.
(60) Provisional application No. 63/433,534, filed on Dec. 19, 2022.
(60) Provisional application No. 63/453,198, filed on Mar. 20, 2023.

(51) Int. Cl. C07K 14/705; A61P 3/04; C07K 14/54; C12N 15/86

(52) U.S. Cl. CPC C07K 14/705; A61P 3/04; C07K 14/5412; C12N 15/86; C12N 2740/15043; C12N 2750/14143

(57) ABSTRACT

Disclosed herein is a readrRNA (RNA sensing by Endogenous ADAR) molecule comprising a modular RNA molecule that facilitates sensing and detection of a cell type or its status, including a cell of a mammalian nervous system, including neurons and/or neuronal cells of the area postrema of the mammalian brain, and/or facilitates delivery of an effector protein to the selected cell. A composition that includes such a modular RNA molecule and another nucleic acid (linked or unlinked to the modular RNA molecule) is a CellREADR (Cell access through RNA sensing by Endogenous ADAR). CellREADR senses the presence of a selected cell RNA in a cell of a mammalian nervous system via readrRNA and leverages RNA editing mediated by ADAR (adenosine deaminase acting on RNA) for coupling the detection of a cell-defining RNA with translation of one or more effector proteins in a cell of a mammalian nervous system.

### CROSS-REFERENCE TO RELATED APPLICATIONS

This application is a continuation application of International Patent Application No. PCT/US2023/084968, filed Dec. 19, 2023, which claims the benefit of U.S. Provisional Patent Application No. 63/433,534 filed Dec. 19, 2022, and U.S. Provisional Patent Application No. 63/453,198, filed Mar. 20, 2023, the contents of each are incorporated herein in their entirety.

### STATEMENT REGARDING FEDERALLY SPONSORED RESEARCH OR DEVELOPMENT

This invention was made with Government support under Federal Grant nos. R01MH101214, R01MH108924, R01NS104944, R01DA050374 awarded by the NIH.

### THE NAMES OF THE PARTIES TO A JOINT RESEARCH AGREEMENT

### Not Applicable

### INCORPORATION-BY-REFERENCE OF MATERIAL SUBMITTED ON A READ-ONLY OPTICAL DISC, AS A TEXT FILE OR AN XML FILE VIA THE PATENT ELECTRONIC SYSTEM

The sequence listing in the attached XML file is hereby incorporated by reference herein in its entirety; the name of the XML file is “123658-12303”, the date of creation of the xml file is “Jun. 17, 2025” and the size of the xml file is 33.2 kb.

### BACKGROUND OF THE INVENTION

The field of the Invention relates to neuronal signalling. More specifically, the invention pertains to Interleukin-6 (IL-6) signaling in neurons in the area postrema (AP) with respect to overeating and cachexia.

Interleukin-6 has long been considered a key player in cancer-associated cachexia¹⁻¹⁵. It is believed that sustained elevation of circulating IL-6 during cancer progression causes brain dysfunctions, which ultimately result in cachexia¹⁶⁻²⁰.

In addition, studies suggest that impaired IL-6 signaling is directly related to obesity⁹⁶. However, how peripheral IL-6 influences the brain and thereby controls both cachexia and obesity is unknown. Further, there are no current methods to specifically control IL-6 signaling in AP neurons in humans.

Cancer-associated cachexia is a devastating metabolic wasting syndrome characterized by anorexia, fatigue, and dramatic involuntary bodyweight loss^{18,19,24,25}. It affects 50-80% of cancer patients, lowering the quality of life, reducing tolerance to anticancer therapies, and drastically accelerating death^{19,20}. The brain is known to have an important role in the pathogenesis of cancer-associated cachexia¹⁶⁻¹⁸. In particular, recent studies implicate the hypothalamus, parabrachial nucleus, area postrema and other hindbrain structures in the development of cachectic phenotypes in animal models of cancer, such as anorexia, weight loss, and accelerated catabolic processes²⁶⁻³². However, how the brain senses and reacts to peripheral cancers, thereby contributing to the development of cachectic phenotypes, is not well understood.

Possible mediators of cancer-associated cachexia that may act as messengers to engage the brain during cancer progression include tumor-derived factors, metabolites from organs indirectly affected by tumor, and immune or inflammatory factors altered by tumor^{16-18,24,33,34} One such messenger is the pleiotropic cytokine IL-6^{18-20,23,24,35,36} Indeed, elevated levels of circulating IL-6 are associated with cancer progression and cachexia in patients and animal models. Systemic administration of antibodies against IL-6 or IL-6 receptor shows anticachectic effects in human case reports^{6,7,37,38} Consistently, cancer-associated cachexia in mouse models can be ameliorated by peripheral administration of antibodies against IL-6⁸⁻¹³ or IL-6 receptor¹⁴, or by deletion of the 116 gene^{9,10}. These findings strongly indicate that IL-6 is a key mediator of cancer-associated cachexia.

Most studies and therapeutic explorations on IL-6 in cancer-associated cachexia have focused on its functions in peripheral organs, including the skeletal muscle, liver, and gut²³ Although previous studies suggest that IL-6 may also influence brain functions-such as the regulation of food intake³⁹⁻⁴¹, fever⁴² and the hypothalamic-pituitary-adrenal (HPA) axis⁴³.

However, it is unclear how peripheral IL-6 is involved in these functions. In principle, IL-6 can activate its receptors on the terminals of peripheral nerves, which then transmit the signals to the brain⁴⁴. Alternatively, circulating IL-6 may cross the blood-brain barrier (BBB) or reach circumventricular organs that lack or have a weak BBB, thereby acting within the brain^{43,45,46}.

### BRIEF SUMMARY OF THE INVENTION

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on studies by the inventors that show that increased IL-6 signaling in neurons in the area postrema (AP) a circumventricular structure in the hindbrain, drives cachexia in tumor-bearing mice while reduction in IL-6 signaling in AP neurons of otherwise healthy mice causes overeating and increased blood glucose, suggesting that IL-6 normally conveys a satiety signal through AP neurons.

The present disclosure comprises a modular RNA molecule comprising, consisting of, or consisting essentially of:
(i) a 5′ region comprising a sensor domain comprising a stretch of consecutive nucleotides that is complementary to a stretch of consecutive nucleotides of a selected cellular RNA of neuron or neuronal cell of the area postrema of the mammalian central nervous systems that encodes the Gfral gene, wherein the sensor domain comprises at least one stop codon editable by ADAR; and
(ii) a 3′ region comprising a domain encoding an effector protein selected from the group consisting of a label, a transcriptional activator, and a transcriptional repressor, wherein the protein coding domain is downstream of and in-frame with the sensor domain, wherein, upon introduction of the modular RNA into the cell of the comprising an Adar enzyme, the stretch of consecutive nucleotides of the sensor domain and the corresponding nucleotide stretch of the cellular RNA form an RNA duplex comprising the stop codon, wherein the stop codon comprised in the RNA duplex is edited by ADAR in the cell, thereby to permit translation of the protein.

In some embodiments, the effector protein comprises a transcription activator that increases the activity of Gfral-expressing (Gfral+) AP neurons. In other embodiments, the transcriptional activator is selected from the group consisting of: IL6a, sodium channel, mutant AMPA receptor, GluA4, and combinations thereof. In some embodiments, the effector protein comprises a sodium channel. In one embodiment, the sodium channel comprises the wild type bacterial Na+ channel (mNaChBac). In another embodiment, the effector protein comprises a mutant AMPA receptor. In one embodiment, the mutant AMPA receptor comprises GluA2-L483Y-R845A.

In another embodiment, the effector protein comprises a transcriptional repressor that decreases the activity of Gfral-expressing (Gfral+) AP neurons. In some embodiments, the transcriptional repressor is selected from the group consisting of: IL6aR, Tetanus Toxin Light Chain (TeLC), a dominant negative Ras, a dominant negative STAT3, GluA4 C-tail, and combinations thereof.

Another embodiment of the present disclosure provides a modular RNA molecule comprising
(i) the 5′ region comprises sensor domains comprising a stretch of consecutive nucleotides of two or more joint sensor domains that are complementary to a stretch of consecutive nucleotides of two or more of a selected cellular RNA of neuron or neuronal cell of the area postrema of the mammalian central nervous system that encodes the Gfral gene, wherein the sensor domain comprises two or more stop codons editable by ADAR; and
(ii) the 3′ region comprises a domain encoding an effector protein selected from the group consisting of a label, a transcriptional activator, and a transcriptional repressor, wherein the protein coding domain is downstream of and in-frame with the sensor domains, wherein, upon introduction of the modular RNA into the cell of the comprising an Adar enzyme, the stretch of consecutive nucleotides of the sensor domains and the corresponding nucleotide stretch of the cellular RNA form an RNA duplex comprising the stop codons, wherein the stop codons comprised in the RNA duplex is edited by ADAR in the cell, thereby to permit translation of the protein.

In some embodiments, the stretch of consecutive nucleotides of the sensor domain is able to form an RNA duplex with at least a portion of an mRNA, the portion comprising a corresponding stretch of consecutive nucleotides.

As used herein, << is able to form a duplex with >> means that the << corresponding stretch of consecutive nucleotides >> can base pair with the stretch of consecutive nucleotides of the sensor domain RNA.

As used herein, “corresponding stretch” means a sequence that is of the same length of nucleotides and matches through base pairing.

“Stretch” indicates a length of consecutive nucleotides that is at least 15 bases or longer; longer includes 20 bases, 25 bases, 30 bases, 40 bases 50 bases, 60 bases, 75 bases, 100 bases, 125 bases, 150 bases, 175 bases, 200 bases, 225, bases, 250 bases, 275 bases, 300 bases, 325 bases, 350 bases, 375 bases, 400 bases, 425 bases, 450 bases, 475 bases, 500 bases, 525 bases, 550 bases, 575 bases, 600 bases, 625 bases, 650 bases, 675 bases, 700 bases, 725 bases, 750 bases, 775 bases, 800 bases, 825 bases, 850 bases, 875 bases, 900 bases, 925 bases, 950 bases, 975 bases, 1000 bases, and longer.

In some embodiments, the effector protein comprises a transcription activator that increases the activity of Gfral-expressing (Gfral+) AP neurons. In other embodiments, the transcriptional activator is selected from the group consisting of: IL6a, sodium channel, mutant AMPA receptor, GluA4, and combinations thereof. In some embodiments, the effector protein comprises a sodium channel. In one embodiment, the sodium channel comprises mNaChBac. In another embodiment, the effector protein comprises a mutant AMPA receptor. In one embodiment, the mutant AMPA receptor comprises GluA2-L483Y-R845A.

In another embodiment, the effector protein comprises a transcriptional repressor that decreases the activity of Gfral-expressing (Gfral+) AP neurons. In some embodiments, the transcriptional repressor is selected from the group consisting of: IL6aR, Tetanus Toxin Light Chain (TeLC), a dominant negative Ras, a dominant negative STAT3, GluA4 C-tail, and combinations thereof.

In some embodiments, the effector protein comprises a Cre recombinase. In some embodiments, the payload comprises a Cas protein. In some embodiments, the payload comprises Cas9. In some embodiments, the payload comprises a transcription factor. In some embodiments, the payload comprises a payload ADAR. In some embodiments, the payload is a reporter for a cellular stress response.

In other embodiments, the molecule further encodes a self-cleaving 2A peptide positioned between the sensor domain and the 3′ protein coding domain. In some embodiments, the self-cleaving 2A peptide is selected from the group consisting of one or more of T2A peptide, P2A peptide, E2A peptide, and F2A peptide.

As used herein, the term “self-cleaving 2A peptide” or “2A peptides” refers to the class of 18-22 amino acid-long peptides which can induce ribosomal skipping during translation of a protein in a cell. These peptides share a core sequence motif of DxExNPGP and are found in a wide range of viral families and help generating polyproteins by causing the ribosome to fail at making a peptide bond. Suitable examples of 2A peptides include, but are not limited to, T2A, P2A, E2A, F2A, and the like (Liu, Ziqing et al. “Systematic comparison of 2A peptides for cloning multi-genes in a polycistronic vector.” Scientific reports vol. 7,1 2193. 19 May. 2017, doi: 10.1038/s41598-017-02460-2). One such self cleaving 2A peptide comprises a T2A peptide.

Several 2A peptides have been identified in picoma viruses, insect viruses and type C rotaviruses. As used herein, T2A is a 2A peptide identified in Thosea asigna virus 2A; P2A is a 2A peptide identified in porcine teschovirus-1 2A; E2A is a 2A peptide identified in equine rhinitis A virus (ERAV) 2A; and F2A is a 2A peptide identified as a self-cleaving 2A peptides foot-and-mouth disease virus (FMDV). The following table provides DNA and corresponding amino acid sequences of representative 2A peptides. Underlined sequences encode amino acids GSG, which are an example of optional additions to the native2A sequence, designed to improve cleavage efficiency; P2A indicates porcine teschovirus-1 2 A; T2A, Thosea Asigna virus 2A; E2A, equine rhinitis A virus (ERAV) 2A; F2A, FMDV 2A. This is adapted from Table 1 of Kim J. H. et al. (High Cleavage Efficiency of a 2A Peptide Derived from Porcine Teschovirus-1 in Human Cell Lines, Zebrafish and Mice) PLOS One. 2011; 6 (4): el8556. Published online 2011 Apr. 29. doi: 10.1371/journal. pone.0018556.

TABLE 1

| P2A | GGA | AGC | GGA | GCT | ACT | AAC | TTC | AGC | CTG | CTG | AAG | CAG | GCT | GGA |
|---|---|---|---|---|---|---|---|---|---|---|---|---|---|---|
| | G | S | G | A | T | N | F | S | L | L | K | Q | A | G |
| | GAC | GTG | GAG | GAG | AAC | CCT | GGA | CCT | SEQ ID NO: 1 | | | | | |
| | D | V | E | E | N | P | G | P | SEQ ID NO: 2 | | | | | |
| T2A | GGA | AGC | GGA | GAC | GGC | AGA | GGA | AGT | CTG | CTA | ACA | TGC | GGT | GAC |
| | G | S | G | E | G | R | G | S | L | L | T | C | G | D |
| | GTC | GAG | GAG | AAT | CCT | GGA | CCT | SEQ ID NO: 23 | | | | | | |
| | V | E | E | N | P | G | P | SEQ ID NO: 24 | | | | | | |
| E2A | GGA | AGC | GGA | CAG | TGT | ACT | AAT | TAT | GCT | CTC | TTG | AAA | TTG | GCT |
| | G | S | G | Q | C | T | N | Y | A | L | L | K | L | A |
| | GGA | GAT | GTT | GAG | AGC | AAC | CCT | GGA | CCT | SEQ ID NO: 25 | | | | |
| | G | D | V | E | S | N | P | G | P | SEQ ID NO: 26 | | | | |
| F2A | GGA | AGC | GGA | GTG | AAA | CAG | ACT | TTG | AAT | TTT | GAC | CTT | CTC | AAG |
| | G | S | G | V | K | Q | T | L | N | F | D | L | L | K |

| | | | | | | | | | | | |
|---|---|---|---|---|---|---|---|---|---|---|---|
| TTG | GCG | GGA | GAC | GTG | GAG | TCC | AAC | CCT | GGA | CCT | SEQ ID NO: 27 |
| L | A | G | D | V | E | S | N | P | G | P | SEQ ID NO: 28 |

As used herein, a “sequence coding for a self-cleaving 2A peptide” is nucleic acid, preferably RNA, encoding a self-cleaving 2A peptide as described above. According to the invention, the sequence coding for a self-cleaving 2A peptide typically is positioned in between the sensor domain and the effector RNA region.

Another aspect of the present disclosure provides a composition comprising, consisting of, or consisting essentially of: i) a first nucleic acid comprising a modular RNA molecule comprising: (a) a sensor domain comprising a stretch of consecutive nucleotides that is complementary to a corresponding stretch of consecutive nucleotides of a selected cellular RNA of neuron or neuronal cell of the area postrema of the mammalian central nervous systems that encodes the Gfral gene,, wherein the sensor domain comprises at least one stop codon editable by ADAR; and (b) a first protein-coding domain encoding an effector protein selected from the group consisting of a label, a transcriptional activator, and a transcriptional repressor, wherein the first protein-coding region is downstream of and in-frame with the sensor domain, and ii) a second nucleic acid comprising a second protein coding domain.

In some embodiments, the first nucleic acid comprises a modular RNA molecule comprising sensor domains comprising a stretch of consecutive nucleotides of two or more joint sensor domains that are complementary to a corresponding stretch of consecutive nucleotides of two or more cellular RNAs, respectively, of a selected cellular RNA of neuron or neuronal cell of the area postrema of the mammalian central nervous systems that encodes the Gfral gene, wherein the sensor domain comprises two or more stop codons editable by ADAR; and the first protein-coding domain encodes an effector protein, wherein the first protein-coding region is downstream of and in-frame with the sensor domains, and ii) the second nucleic acid comprises a second protein coding domain.

In some embodiments, the first and second nucleic acids comprise a single nucleic acid molecule.

In another embodiment, the first and second nucleic acids comprise two nucleic acid molecules.

In yet other embodiments the first and second nucleic acid are covalently linked.

In some embodiments, the effector protein comprises a transcription activator that increases the activity of Gfral-expressing (Gfral+) AP neurons. In other embodiments, the transcriptional activator is selected from the group consisting of: IL6a, sodium channel, mutant AMPA receptor, GluA4, and combinations thereof. In some embodiments, the effector protein comprises a sodium channel. In one embodiment, the sodium channel comprises mNaChBac. In another embodiment, the effector protein comprises a mutant AMPA receptor. In one embodiment, the mutant AMPA receptor comprises GluA2-L483Y-R845A.

In another embodiment, the effector protein comprises a transcriptional repressor that decreases the activity of Gfral-expressing (Gfral+) AP neurons. In some embodiments, the transcriptional repressor is selected from the group consisting of: IL6aR, Tetanus Toxin Light Chain (TeLC), a dominant negative Ras, a dominant negative STAT3, GluA4 C-tail, and combinations thereof.

Another aspect of the present disclosure provides a nucleic acid delivery vehicle comprising, consisting of, or consisting essentially of the modular RNA molecule as provided herein, the composition as provided herein, and/or DNA encoding the modular RNA molecule as provided herein or the composition as provided herein.

In other embodiments, the delivery vehicle is selected from the group consisting of a nanoparticle, a liposome, a LNP, a vector, an exosome, a micro-vesicle, a gene-gun, and a Selective Endogenous encapsulation for cellular Delivery (SEND) system.

In some embodiments, the delivery vehicle comprises a viral vector. In one embodiment, the viral vector is selected from the group consisting of adeno-associated virus (AAV), adenovirus, retrovirus, lentivirus, herpes virus, vesicular stomatitis virus.

In another embodiment the modular RNA molecule, composition comprising the modular RNA molecule and/or the delivery vehicle comprising the modular RNA molecule or composition thereof is encoded by a DNA vector.

Another aspect of the present disclosure provides a pharmaceutical composition comprising, consisting of, or consisting essentially of the modular RNA molecule as provided herein, the composition as provided herein, or the delivery vehicle as provided herein, and a pharmaceutically acceptable carrier, excipient and/or diluent.

Another aspect of the present disclosure provides a cell comprising, consisting of, or consisting essentially of the modular RNA molecule as provided herein, the composition as provided herein, or the delivery vehicle as provided herein. In some embodiments, the cell is a mammalian cell.

Another aspect of the present disclosure provides a kit comprising, consisting of, or consisting essentially of the modular RNA molecule as provided herein, the composition as provided herein, or the delivery vehicle as provided herein and packaging therefore.

Another aspect of the present disclosure provides a method for treating a disease or disorder in a mammal, the method comprising, consisting of, or consisting essentially of administering to a subject in need thereof a therapeutically effective amount of the modular RNA molecule as provided herein, the composition as provided herein, or the delivery vehicle as provided herein or a pharmaceutical composition thereof to permit translation of the 3′ encoded protein or the effector protein in selected cells of the subject, thereby to produce the protein in the cells, wherein production of the protein in the cells provides for treatment of the disease or disorder in the mammal.

In one embodiment, the disease or disorder is selected from the group consisting of obesity and cachexia.

Another aspect of the present disclosure provides all that is described and illustrated herein.

### BRIEF DESCRIPTION OF THE DRAWING(S)

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIG. 1a-FIG. 1j illustrate that circulating IL-6 can reach the area postrema (AP) and activate AP neurons, in accordance with one embodiment of the present disclosure. FIG. 1a. A schematic of the approach. FIG. 1b. Confocal images showing the binding of the exogenous IL-6 to cells in the AP. FIG. 1c. Quantification of the fluorescence signals from fluorescence-conjugated avidin in the AP, which recognizes the biotinylated exogenous IL-6 (n=3 mice for saline and IL-6 baseline group, n=4 mice for IL-6 cachexia group. F=37.68, p=0.0002, *P=0.0212, **P=0.002, ***P=0.001, one-way ANOVA followed by Tukey's multiple comparison test.). FIG. 1d. A diagram showing the position of the AP in a coronal brain section. FIG. 1e. Confocal images showing Fos expression in the AP. FIG. 1f. Quantification of Fos-expressing (Fos) cells in the AP (n=3 mice for saline and IL-6 baseline group, n=4 mice for IL-6 cachexia group, F=71.02, P=0.000022, *P=0.0103, ***P=0.00044, ****P=0.000017, one-way ANOVA followed by Tukey's multiple comparison test.). FIG. 1g. Confocal images showing the expression of different genes in AP cells, which was detected with single molecule fluorescent in situ hybridization (smFISH). At the bottom are higher magnification images of the boxed area in the overlay image on the top. Arrowheads indicate a neuron that expresses all three genes. FIG. 1h. A Venn diagram showing the relationships among cells expressing Il6ra, Gfral, and Glp1r in the AP. FIG. 1i. Characterization of the types of Fos⁺ cells in the AP by smFISH. At the bottom are higher magnification images of the boxed areas in images on the top. FIG. 1j. A Venn diagram showing the relationships among cells expressing Fos, Il6ra, and Glp1r in the AP.

FIG. 2a-2i illustrate that C26 cancer causes increased IL-6 in the AP and AP neuron hyperactivity, in accordance with one embodiment of the present disclosure. FIG. 2a. A schematic of the experimental procedure. FIG. 2b. IL-6 levels in the area postrema (AP) during cancer progression. IL-6 levels were normalized to total protein levels (n=8-10 mice in each group, For IL-6, F=5.883, P=0.0024, *P=0.011, *P=0.0483, **P=0.001; For total protein level, F=0.61, P=0.61. One-way ANOVA followed by Tukey's post-hoc test). FIG. 2c. Confocal images showing Fos expression in different brain areas in tumor-bearing (top) or control (bottom) mice. FIG. 2d. Quantification of Fos cells in different brain areas (n=4 mice in each group; AP, t=2.91, *P=0.027, NTS, T=9.77, ****P=6.62×10⁻⁵, PBN, t=11.65, ****P=2.41×10⁻⁵, CeA, t=4.37, **P=0.0047, PVN, t=3.65, *P=0.011, BNST, t=5.86, **P=0.0011; unpaired two-tailed t test). FIG. 2e. A diagram showing the AP in a coronal brain section for electrophysiological recording. FIG. 2f. Representative miniature EPSC traces from AP neurons in control (top) and cachectic (bottom) mice. FIG. 2g. Quantification of miniature EPSC frequency (left) and amplitude (right) (control, n=30 cells/6 mice, cachexia, n=32 cells/7 mice; frequency, n.s. (nonsignificant), P=0.2513, amplitude, **P=0.0017, Mann-Whitney test). FIG. 2h. Representative spontaneous IPSC traces from AP neurons in control (top) and cachectic (bottom) mice. FIG. 2i. Quantification of spontaneous IPSC frequency (left) and amplitude (right) (control, n=36 cells/7 mice, cachexia, n=34 cells/6 mice; frequency, n.s., P=0.1637, amplitude, n.s., P=0.4580, Mann-Whitney test).

FIG. 3a-FIG. 3g illustrate that Intracerebroventricular (i.e.v.) infusion of IL-6 antibody prevents cachexia in the C26 cancer model, in accordance with one embodiment of the present disclosure. FIG. 3a. A schematic of the experimental procedure. FIG. 3b. Average bodyweight normalized to that on day-5 (IL-6 antibody, n=6, isotype control antibody, n=6; F(1,60)=28.46, ****P=1.53×10⁻⁶, two-way repeated-measures (RM) ANOVA followed by Sidak's post hoc test). FIG. 3c and FIG. 3d. Cumulative food (c) and water (d) intake of the mice in the 5 days before euthanization (n=6 animals in each group, c, F(1,60)=25.96, ****P=3.73×10⁻⁶. d, F(1,60)-45.25, ****P=7.3×10⁻⁹, two-way repeated-measures (RM) ANOVA followed by Sidak's post hoc test). FIG. 3e. Comparison of different tissue mass between IL-6 antibody pretreatment group and isotype antibody pretreatment group (n=6 mice in each group, muscle, t=5.72, ***P=0.0002; fat, t=4.74, ***P=0.0008; tumor, t=0.18, P=0.858; spleen, t=0.4, P=0.7; glucose, t=5.66, ***P=0.0002; unpaired two-tailed t test.). FIG. 3f. Confocal images showing Fos expression in different brain areas in the mice infused with the isotype antibody (top) and the IL-6 antibody (bottom). FIG. 3g. Quantification of Fos+ cells in different brain areas (n=6 mice in each group, AP, t=7.03, ****P=3.6×10⁻⁵; NTS, t=3.87, **P=0.003; PBN, t=4.2, **P=0.0018; PVN, t=5.62, ***P=0.0002; CeA, t=4.08, **P=0.0022; BNST, t=5.21, ***P=0.0004; unpaired two-tailed t test.). Scale bar in f is 100 μm.

FIG. 4a-FIG. 4h illustrate that suppression of Il-6ra expression in AP neurons ameliorates cachexia in the C26 cancer model, in accordance with one embodiment of the present disclosure. FIG. 4a. A schematic of the experimental procedure. FIG. 4b. Confocal immunohistochemical images of a coronal brain section from a representative mouse, showing the infection of AP cells with lentiviruses expressing the sgRNA (tagged with mCherry) and dCas9-KRAB-MeCP2 (tagged with FLAG). mCherry and FLAG were recognized by antibodies. The arrowheads indicate the dual-color labeled cells. FIG. 4c. Survival curves of the mice after tumor inoculation (lacZ sgRNA group, n=8, Il6ra sgRNA-4 group, n=7; P<0.0001, Mantel-Cox test). FIG. 4d. Relationship between survival days and the fractions of Gfral⁺ neurons expressing Il6ra (n=17 mice, R2=0.806, P<0.0001 by a linear regression). FIG. 4e. Bodyweight of individual mice relative to their bodyweight on the day of tumor inoculation. FIG. 4f. Average bodyweight normalized to that on day-5 (lacZ sgRNA group, n=8, Il6ra sgRNA-4 group, n=7; F(1,78)=23.63, P=9.6×10⁻⁶, *P=0.012, ****P=0.00004, two-way repeated-measures (RM) ANOVA with Sidak's post hoc test). FIG. 4g. Cumulative food (left) and water (right) intake of the mice after tumor inoculation (food: F(1,130)=46.9, P=2.67×10⁻¹⁰; day 8, *P=0.037, day 9, **P=0.0076, day 10, **P=0.005; water: F(1,130)=43.87, P=8.41×10⁻¹⁰; day 8, *P=0.0424, day 9, **P=0.008, day 10, **P=0.0011; two-way RM ANOVA with Sidak's post hoc test). FIG. 4h. Cumulative food (left) and water (right) intake of the mice in the 5 days before euthanization (food: F(1, 65)=12.82, P=0.0007; water: F(1, 65)=16.37, P=0.00014; two-way RM ANOVA with Sidak's post hoc test).

FIG. 5a-FIG. 5e illustrates suppression of Il6ra expression in AP neurons ameliorates cachexia in the pancreatic cancer model, in accordance with one embodiment of the present disclosure. FIG. 5a. A schematic of the experimental procedure. FIG. 5b. Normalized bodyweight, food intake and water intake after tumor implantation. The bodyweight was normalized to the initial bodyweight when tumor was implanted. Bodyweight, F(1,130)-0.0021, P=0.96; food intake, F(1,120)-28.28, ****P=4.93×10⁻⁷; water intake, F(1,120)=8.46, **P=0.0043. n=5 mice in control group, n=7 mice in Il6ra knock down group. FIG. 5c. Comparison of different tissue mass between control group and Il6ra knock down group, n=5 mice in control group, n=7 mice in Il6ra knock down group. Muscle, t=5.51, ***P=0.0003; fat, t=4.37, **P=0.0014; tumor, t=0.26, P=0.8014; spleen, t=0.574, P=0.5786. FIG. 5d. Confocal images showing Fos expression in different brain areas in control group (top) and in Il6ra knock down group (bottom). FIG. 5e. Quantification of cfos expression in different brain areas. AP, t=7.58, ****P=1.88×10⁻⁵; NTS, t=2.07, P=0.0656; PBN, t=6.78, ****P=4.84×10⁻⁵; PVN, t=4.4, **P=0.0013; CeA, t=4.64, ***P=0.0009; BNST, t=3.41, **P=0.0066. Unpaired two-tailed t test. Scale bar in d is 100 μm.

FIG. 6a-FIG. 6h illustrate that Inhibition of Gfral+AP neurons ameliorates cachexia in the Lewis lung cancer (LLC) mode, in accordance with one embodiment of the present disclosure. FIG. 6a. Plasma IL-6 (left) and GDF-15 (right) concentrations during cancer progression (IL-6: control, 7 mice at all timepoints except day 2 and day 8, where there are 5 mice; LLC, 8 mice at all timepoints except day 4 where there are 6 mice, day 6 and day 12 where there are 11 mice, and day 10 and day 18 where there are 7 mice; *P<0.05; GDF-15: control, 5 mice at all timepoints except day 0 where there are 8 mice, and day 14 where there are 3 mice; LLC, 5 mice at all timepoints except day 0 where there are 7 mice; *P<0.05, **P<0.01; multiple unpaired t-tests at each timepoint with false discovery rate adjusted with the two-stage step-up method). FIG. 6b. A schematic of the experimental procedure. FIG. 6c. Confocal immunohistochemical images of coronal brain sections from two representative mice, showing the infection of Gfral⁺ AP neurons with an AAV expressing TeLC (left) or GFP only (right). FIG. 6d. Cumulative food (left) and water (right) intake of the mice in the 7 days before being euthanized (TeLC tumor group, n=9, GFP tumor group, n=8; GFP sham group, n=6; food: F(2,160)=98.25, P=10-15; day-4, TeLC tumor group vs GFP tumor group, ***P=0.0001; GFP tumor group vs GFP sham group, **P=0.003; day-3, TeLC tumor group vs GFP tumor group, ****P=7.07×10⁻⁷, GFP tumor group vs GFP sham group, ****P=5.5×10⁻⁵; day-2, TeLC tumor group vs GFP tumor group, ****P=2.08×10⁻¹⁰, GFP tumor group vs GFP sham group, ****P=5.9×10⁻⁸; day-1, TeLC tumor group vs GFP tumor group, ****P=7.93×10⁻¹³, GFP tumor group vs GFP sham group, ****P=4.17×10⁻¹³; day 0, TeLC tumor group vs GFP tumor group, ****P=2.7×10⁻¹⁴, GFP tumor group vs GFP sham group, ****P=10-15, Telc tumor group vs GFP sham group, *P=0.048; water: F(2,160)=2.889, P=0.06; two-way repeated-measures ANOVA with Sidak's post hoc test. FIG. 6e. Inguinal fat (left) and quadriceps muscle (right) mass of the mice at 23 days after tumor inoculation (TeLC group, n=9, GFP group, n=8; fat: t=2.3, *P=0.0363; muscle: t=4.04, **P=0.001; t test). FIG. 6f. Confocal images showing Fos expression in different brain areas in the mice where Gfral⁺ AP neurons were infected with the AAV expressing TeLC (top) or GFP only (bottom). FIG. 6g. Quantification of Fos cells in different brain areas (TeLC group, n=9 mice, GFP group, n=8 mice; AP, t=2.33, *P=0.0343; NTS, t=3.3, **P=0.0049; PBN, t=4.04, **P=0.0011; PVN, t=3.09, **P=0.0094; CeA, t=2.62, *P=0.0193; BNST, t=2.56, *P=0.022; t test with false discovery rate adjusted). FIG. 6h. Tumor (left) and spleen (right) mass of the mice at 23 days after tumor inoculation (TeLC group, n=9, GFP group, n=8; tumor: t=1.34, P=0.2; spleen: t=0.65, P=0.525; t test).

FIG. 7a-FIG. 7d illustrate Targeting area postrema neurons with the Gfral sensor for the treatment of cancer cachexia. FIG. 7a. A schematic of the approach to test the specificity of the Gfral sensor. The Gfral sensor AAV expressing tTA was delivered through retro-orbital injection. A tTA dependent AAV expressing GFP was injected into the area postrema (AP). FIG. 7b. Confocal immunohistochemical images of a coronal brain section containing the AP, which was from a representative mouse prepared as in A. The expression of GFP (upper right) and Gfral (lower left) was detected with antibodies against GFP and Gfral, respectively. The vast majority of GFP-expressing neurons also expressed Gfral (overlay in lower right). FIG. 7c. Quantification of the overlap between GFP and Gfral. 94% (103 out of 110 cells) of GFP-expressing neurons also expressed Gfral. FIG. 7d. The relative bodyweight (left), cumulative food intake (middle) and water intake (right) of two mice, with one expressing GFP (the control mouse) and the other expressing tetanus toxin light chain (TeLc, the experimental mouse) in AP Gfral+ neurons, which were targeted with the Gfral sensor. Both mice were inoculated with C26 tumor after the virus injections as shown in A. The TeLc mouse showed higher body weight, and higher food and water intake than the GFP mouse.

FIG. 8A illustrate that ses^{RNAGfral=3} and ses^{RNAGfral=4} comprising SEQ ID NO:s 14 and 15, respectively, show especially robust Gfral targeting efficiency and specificity for Gfral mRNA in HEK cells according to the method described in Part A of Working Example 1. Blue fluorescence is expressed from the READR construct in HEK cells with or without transfected Gfral target, while green florescence is expressed only in HEK cells with transfected Gfral target as a result of the target Gfral RNA hybridizing with the SES component of the READR construct, which triggers ADARs mediated A->I editing, converting the UAG STOP in the READR construct to a UGG Trp codon, switching on translation of the green fluorescent (GFP) effector protein.

FIG. 8B illustrates successful CellReadr mediated targeting of Gfral neurons in mouse area postrema as described in Working Example 1. FIG. 8B 300 ng of a sesRNA^{Gfral#}1, #2, #3, #4, #5, #6, #7 or #8 comprising SEQ ID NO:s 4-11, respectively, function in CellReadr mediated targeting and detection of Gfral neurons in mouse area postrema, with sesRNA^{Gfral#}4, 7 and 8 demonstrating especially robust staining. The relevant sequences of the Gfral sesRNAs used to target the 150 ng of mouse Gfral RNA (SEQ ID NO:3) in vitro are disclosed in Working Example 1.

Data of FIG. 9a-FIG. 9b illustrate that Circulating IL-6 does not reach brain areas other than the AP, in accordance with one embodiment of the present disclosure. Data of FIG. 9a. Confocal images showing the lack of exogenous IL-6 signals in different brain areas of mice received biotinylated IL-6 (left) compared with mice received saline (right) via retro-orbital injection. Data of FIG. 9b. Quantification of the fluorescence signals from fluorescence-conjugated avidin in different brain areas, which recognizes the biotinylated exogenous IL-6 (n=3 mice in saline group and IL-6 baseline group, n=4 mice in IL-6 cachexia group; LS, F=3.605, P=0.084; BNST, F=2.54, P=0.15; PVN, F=3.57, P=0.086; CeA, F=3.73, P=0.08; ME, F=0.55, P=0.6; PBN, F=0.66, P=0.55; NTS, F=0.0012, P=0.98. One-way ANOVA followed by Tukey's multiple comparison test.

Data of FIG. 10a-data of FIG. 10b illustrate that Intracerebroventricular (i.e.v.) infusion of IL-6 antibody improves the physiological conditions of mice despite tumor growth in the C26 cancer model, in accordance with one embodiment of the present disclosure. Data of FIG. 10a. Confocal images showing the cfos expression in different brain areas. Data of FIG. 10b. (n=3 mice in saline group and IL-6 baseline group, n=4 mice in IL-6 cachexia group; LS, F=0.5, P=0.63; BNST, F=21, P=0.0011, **P=0.001, **P=0.0055; PVN, F=66.65, P=0.00003, ****P=0.00005, ****P=0.00005; CeA, F=20.52, P=0.0012, **P=0.0011, **P=0.0052; ME, F=0.55, P=0.6; PBN, F=30.25, P=0.0004, ***P=0.0003, **P=0.002; NTS, F=30.15, P=0.0004, **P=0.0033, ***P=0.0003.

Data of FIG. 11a-Data of FIG. 11b illustrate confocal images showing the expression of different genes in AP and NTS cells, in accordance with one embodiment of the present disclosure. Data of FIG. 11a. The gene expression of Glp1r, Gfral and Il6ra in the AP and NTS. Data of FIG. 11b. The gene expression of Glp1r, Il6ra and cfos in the AP and NTS after IL-6 injection. Scale bars in a and b are 100 μm.

Data of FIG. 12a-Data of FIG. 12b illustrate the IL-6 level in the ME (left) and cortex (right) during C26 tumor progression, in accordance with one embodiment of the present disclosure. Data of FIG. 12a. IL-6 levels in the median eminence (ME) during cancer progression. IL-6 levels were normalized to total protein levels (n=7 mice in each group; Balb/C WT vs C26/Cx F=3.892, *P=0.0334; C26/Day 12 vs C26/Cx F=5.305, **P=0.0040; One-way ANOVA followed by Tukey's post-hoc test). Data of FIG. 12b. IL-6 levels in the cortex during cancer progression. IL-6 levels were normalized to total protein levels (n=7 mice in each group; One-way ANOVA followed by Tukey's post-hoc test).

Data of FIG. 13a-Data of FIG. 13h illustrate that suppression of Il6ra expression in AP neurons ameliorates cachexia and reduces the hyperactivity in the AP network in the C26 cancer model, in accordance with one embodiment of the present disclosure. Data of FIG. 13a. A schematic of the experimental procedure. Data of FIG. 13b. A confocal image of a coronal brain section from a representative mouse, showing the location of the infusion cannula above the lateral ventricle (VL). Data of FIG. 13c. Survival curves of the mice after tumor inoculation (IL-6 antibody group, n=10, isotype control antibody group, n=6; P=0.0003, Mantel-Cox test). Data of FIG. 13d. Bodyweight of individual mice relative to their bodyweight on the day of tumor inoculation. Data of FIG. 13e. Average bodyweight normalized to that on day-5 (IL-6 antibody, n=10, isotype control antibody, n=6; F(1,84)=75.13, P=2.77×10⁻¹³; day-1, ****P=1.2×10⁻⁶, day 0, ****P<1×10⁻¹⁵; two-way repeated-measures (RM) ANOVA followed by Sidak's post hoc test). Data of FIG. 13f. Cumulative food (left) and water (right) intake of the mice in the 5 days before sacrifice (IL-6 antibody, n=10, isotype control antibody, n=6 mice; food: F(1,70)=42.45, P=9.44×10⁻⁹, day-1, ***P=0.0007, day 0, ****P=0.00003; water: F(1,70)=112.2, P<1×10⁻¹⁵, day-2, **P=0.003, day-1, ****P=1.13×10⁻⁹, day 0, ****P=1.33×10⁻¹²; two-way RM ANOVA with Sidak's post hoc test). Data of FIG. 13g. Confocal images showing Fos expression in different brain areas in the mice infused with the IL-6 antibody (top) and the control antibody (bottom). Data of FIG. 13h. Quantification of Fos cells in different brain areas (IL-6 antibody, n=9 mice, isotype control antibody, n=6 mice; AP, t=8.11, ****P=1.83×10⁻¹¹, NTS, t=1.62, P=0.12, PBN, t=0.8213, P=0.41, CeA, t=0.1375, P=0.89, PVN, t=15.55, ****P<10-15, BNST, t=2.302, *P=0.0245; t test with false discovery rate adjusted).

Data of FIG. 14a-Data of FIG. 14f illustrate that intracerebroventricular (i.e.v.) infusion of IL-6 antibody improves the physiological conditions of mice despite tumor growth in the C26 cancer model, in accordance with one embodiment of the present disclosure. Data of FIG. 14a. Inguinal fat mass (anti-IL-6 group, n=10, isotype control antibody group, n=6; t=2.334, *P=0.035, t test). Data of FIG. 14b. Quadriceps muscle mass (anti-IL-6 group, n=10, isotype control antibody group, n=6; t=2.538, *P=0.0237, t test). Data of FIG. 14c. Blood glucose levels at the endpoint (anti-IL-6 group, n=9 mice, isotype control antibody group, n=6 mice; t=3.555, **P=0.0035, t test). Data of FIG. 14d. IL-6 levels in the plasma (left) and cerebrospinal fluid (CSF; right). Plasma IL-6: anti-IL-6 group, n=9 mice, isotype control antibody group, n=8 mice; t=1.664, P=0.1168; CSF IL-6: anti-IL-6 group, n=8 mice, isotype control antibody group, n=7 mice; t=2.002, P=0.0666; t test. Data of FIG. 14e. Tumor mass (anti-IL-6 group, n=10, isotype control antibody group, n=6; t=2.864, *P=0.0125, t test). Data of FIG. 14f. Spleen mass (anti-IL-6 group, n=10, isotype control antibody group, n=6; t=3.274, **P=0.0055, t test).

Data of FIG. 15a-Data of FIG. 15b illustrate the design and characterization of sgRNAs for the CRISPR/dCas9 system to suppress Il6ra expression, in accordance with one embodiment of the present disclosure. Data of FIG. 15a. Transcription start site (TSS) targeting positions of the different sgRNAs. Cyan, template strand; red, non-template strand. Data of FIG. 15b. In vitro characterization of the efficacy of the CRISPR/dCas9 system with different sgRNAs. Plasmids expressing dCas9-KRAB-MeCP2 and each of the sgRNAs were co-transfected into mHypoA cell line. 60 hours after the transfection, the expression of Il6ra in these cells was measured by qPCR (Il6ra sgRNA-4, n=6 plates, lacZ sgRNA, n=6 plates, P=0.0005785; Il6ra sgRNA-6, n=3 plates, lacZ sgRNA, n=3 plates, P=0.1965; t test).

Data of FIG. 16a-Data of FIG. 16d illustrate in vivo validation of Il6ra knock down virus, in accordance with one embodiment of the present disclosure. Data of FIG. 16a. Confocal images showing the colocalization between NeuN and mCherry. Data of FIG. 16b. Quantification of the colocalization between NeuN and mCherry. n=4 mice. Data FIG. 16c. Confocal images showing the expression of Il6ra, Gfral and mCherry expression in control group and Il6ra knock down group. Data of FIG. 16d. Quantification of the fractions of Gfral⁺ neurons expressing Il6ra in the AP of individual mice (lacZ sgRNA group, n=4, Il6ra sgRNA-4 group, n=4; t=22.43, ****P=2.13×10⁻⁷, t test).

Data of FIG. 17a-Data of FIG. 17e illustrate suppression of Il6ra expression in AP neurons improves the physiological conditions of mice despite tumor growth in the C26 cancer model, in accordance with one embodiment of the present disclosure. Il6ra sgRNA-4 group, n=7 mice, lacZ sgRNA group, n=8 mice. Data of FIG. 17a. Blood glucose levels at different time points (F(1,49)=9.834, P=0.0029; two-way repeated-measures ANOVA with Sidak's post hoc test). Data of FIG. 17b. Inguinal fat mass (t=1.657, P=0.12, t test). Data of FIG. 17c. Quadriceps muscle mass (t=1.92, P=0.078, t test). Data of FIG. 17d. Tumor mass (t=7.32, ****P=5.86×10⁻⁶, t test). Data of FIG. 17e. Spleen mass (t=2.71, P=0.018, t test).

Data of FIG. 18a-Data of FIG. 18j, illustrate suppression of Il6ra expression in AP neurons ameliorates cachexia and reduces the hyperactivity in the AP network in the C26 cancer model, in accordance with one embodiment of the present disclosure. Data of FIG. 18a. A schematic of the experimental procedure. When one animal in the lacZ sgRNA (control) group became cachectic, that animal and a randomly selected animal in the Il6ra sgRNA-4 group were sacrificed to check Fos expression and other phenotypes. Data of FIG. 18b. Average bodyweight normalized to that on day-5 (F(1,36)=10.45, P=0.0026, **P=0.0072, two-way repeated-measures (RM) ANOVA with Sidak's post hoc test, lacZ sgRNA group, n=4 mice, Il6ra sgRNA-4 group, n=4 mice). Data of FIG. 18c. Inguinal fat mass (t=3.941, **P=0.0079, t test). Data of FIG. 18d. Quadriceps muscle mass (t=5.26, **P=0.0019, t test). Data of FIG. 18e, Data of FIG. 18f. Cumulative food (e) and water (f) intake of the mice in the 5 days before sacrifice (food: F(1, 36)=0.31, P=0.58; water: F(1, 36)=0.81, P=0.37. two-way RM ANOVA with Sidak's post hoc test). Data of FIG. 18g, Data of FIG. 18h. Tumor (g) and spleen (h) mass of the mice (tumor: t=0.444, P=0.6725; spleen: t=0.898, P=0.404; t test). Data of FIG. 18i. Confocal images showing Fos expression in different brain areas in representative mice of the two groups. Data of FIG. 18j. Quantification of Fos cells in different brain areas (AP, t=4.617, **P=0.0036; NTS, t=1.5, P=0.18; PBN, t=2.849, *P=0.029; PVN, t=5.332, **P=0.0018; CeA, t=0.5267, P=0.617; BNST, t=0.27, P=0.796; t test with false discovery rate adjusted).

Data of FIG. 19a-Data of FIG. 19j illustrates suppression of Il6ra expression in AP neurons ameliorates cachexia in the C26 cancer model, in accordance with one embodiment of the present disclosure. Il6ra sgRNA-6 group, n=5 mice, lacZ sgRNA group, n=7 mice. Data of FIG. 19a. Survival curves of the mice after tumor inoculation (P=0.0008, Mantel-Cox test). Data of FIG. 19b. Bodyweight of individual mice relative to their bodyweight on the day of tumor inoculation. Data of FIG. 19c. Average of bodyweight normalized to that on day-5 (F(1,60)=8.287, P=0.0055; day 0, *P=0.011, two-way repeated-measures (RM) ANOVA with Sidak's post hoc test). Data of FIG. 19d. Cumulative food (left) and water (right) intake of the mice after tumor inoculation (food: F(1,100)=30.28, P=2.89×10⁻⁷, *P=0.02; water: F(1,100)=5.146, P=0.0255; two-way RM ANOVA with Sidak's post hoc test). Data of FIG. 19e. Cumulative food (left) and water (right) intake of the mice in the 4 days before sacrifice (food: F(1,50)=2.051, P=0.158; water: Data of FIG. 19F (1,50)=6.396, P=0.0146; two-way RM ANOVA with Sidak's post hoc test). Data of FIG. 19f. Blood glucose levels at different time points (F(1,38)=29.7, P=3.24×10⁻⁶, *P=0.013, **P=0.0056, two-way RM ANOVA with Sidak's post hoc test). Data of FIG. 19g. Inguinal fat mass (t=2.78, *P=0.0195, t test). Data of FIG. 19h. Quadriceps muscle mass (t=4.65, ***P=0.0009, t test). Data of FIG. 19i. Tumor mass (t=7, ****P=0.000037, t test). Data of FIG. 19j. Spleen mass (t=3.43, **P=0.0064, t test).

Data of FIG. 20a-Data of FIG. 20f illustrate characterization of cachectic phenotype in pancreatic tumor model, in accordance with one embodiment of the present disclosure. Data of FIG. 20a-Data of FIG. 20c. Normalized bodyweight, food intake and water intake in tumor bearing mice and control mice. Bodyweight, F(1,96)=1.098, P=0.3; food intake, F(1,96)=95.33, P<10-15; water intake, F(1,96)-22.43, P=7.53×10⁻⁶. Data of FIG. 20d. Comparison of plasma IL-6 level, muscle, fat and spleen in tumor bearing mice and control mice. IL-6, t=3.250, *P=0.0117; muscle, t=5.41, ***P=0.0006; fat, t=4.87, **P=0.0012; spleen, t=3.91, **P=0.0045. Data of FIG. 20e. Confocal images showing the cfos expression in different brain areas in tumor bearing mice and control mice. Data of FIG. 20f. Quantification of cfos expression in different brain areas. AP, t=8.52, ****P=0.00003; NTS, t=8.64, ****P=2.5×10⁻⁵; PBN, t=7.58, ****P=6.44×10⁻⁵; PVN, t=9.47, ****P=0.00001; CeA, t=2.73, *P=0.0257; BNST, t=5.2, ***P=0.0008. Tumor group, n=4 mice, sham group, n=6 mice.

FIG. 21 The 11 sesRNAs were designed to target mouse Gfral coding RNA sequence. Each line across indicates one sesRNA targeting region to Gfral coding sequences. Some sesRNAs contain spreading fragments for multiple targeting, with one or two stops in the middle fragment.

FIG. 22 illustrates a procedure to screen the sensors for Gfral mRNA in HEK cells, where READR^{Gfral-GFP} encodes a readrRNA consisting of a BFP (Blue Fluorescent Protein) sequence followed by sesRNA^{Gfral} and an effector protein, in this case green fluorescent protein (efRNAGFP).

FIG. 23 is a schematic of experimental procedures for sesRNA^{Gfral} screening.

FIG. 24 illustrates a luciferase assay was used for quantitative measurement of sesRNA^{Gfral} efficacy and specificity.

FIG. 25 illustrate results that indicate that sesRNA^{Gfral#}6 and sesRNA^{Gfral#}4 show the best Gfral targeting efficiency and specificity with in vitro transcription luciferase assay.

FIG. 26 is a schematic of binary adeno-associated virus (AAV) vectors for targeting Gfral neurons includes a READR vector, a human synapsin (hSyn) promoter which drives transcription of ClipF (a CLIP-tag protein) followed by sesRNA (#4 or #6) and efRNA encoding an smFlag tag which is surrounded by 2A self cleving peptide, followed by tTA2 (modified tetracycline-regulated transactivator) and W3SL (a modified WPRE/polyA sequence). The Reporter vector contains a TRE3g promoter (provides very tight control of transcription) driving mNeonGreen fluorescent protein (mNeon) and a woodchuck hepatitis virus posttranscriptional regulatory element (WPRE).

FIG. 27 illustrates a strategy to increase the activity of Gfral-expressing (Gfral+) neurons in the area postrema for the treatment of obesity.

FIG. 28 illustrates a strategy to decrease the activity of Gfral+ neurons in the area postrema for the treatment of cancer cachexia.

FIG. 29 illustrates the genomic locus of the mouse Gfral which is 49,556 base pairs, with a coding sequence of 1182 base pairs.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. Other than in the operating examples, or where otherwise indicated, all numbers expressing quantities of ingredients or reaction conditions used herein should be understood as modified in all instances by the term “about.” The term “about” when used in connection with percentages can mean±1%.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional character! stic(s) of that embodiment of the invention.

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

Unless otherwise defined, all technical terms used herein have the same meaning as commonly understood by one of ordinary skill in the art to which this disclosure belongs.

The present disclosure provides, in part, methods of treating and/or preventing obesity and/or cachexia (including cancer cachexia) using a new class of cell type technology that bypasses DNA-based transcriptional process and directly engages cell type-defining RNAs for use in the bidirectional regulation of IL-6 signaling or neuronal excitability. The methods provided herein utilize a dual function RNA molecule (readrRNA) which permits (i) targeting of a selected somatic cell based on its transcript profile and (ii) translation in the selected cell of a desired effector protein encoded by the RNA molecule, translation of the effector protein being implemented through RNA editing mediated by adenosine deaminase acting on RNA (ADAR) and the use of said system for the treatment and/or prevention of cachexia and obesity in a subject.

### A. ADAR-Mediated RNA Editing

RNA editing is a widespread post-transcriptional process that alters the sequence of RNA encoded by the DNA template, ubiquitous in all metazoan cells. Across the animal kingdom, the most prevalent form of RNA editing is adenosine-to-inosine (A-to-I) conversion, catalyzed by the ADAR (adenosine deaminase acting on RNA) family of enzymes, which has three members in mammals (ADAR1, ADAR2, ADAR3). The edited inosine then base pairs, instead, with cysteine, and is recognized as guanosine (G) by various cellular machinery. ADAR-mediated A-I editing is ubiquitous to all metazoan cells.

In some embodiments, the ADAR is selected from the group consisting of ADAR2, ADAR1, ADAR1 p150, ADAR1 p110, ADAR2 R455G, ADAR2 R455G, ADAR2 S486T, ADAR2 T375G E488Q T490A, ADAR2 T375G, ADAR2 T375S, ADAR2 N473D, ADAR2 deaminase domain, ADAR2 T490S, ADAR2 T490A, MCP-ADAR2 deaminase domain, ADAR2 R455E, ADAR2 T375G T490A, ADAR2 E488Q, MCP-ADAR2 deaminase domain E488Q T490A, ADAR2 R510E, ADAR2 R455S, ADAR2 V351L, and derivatives thereof. In some embodiments the ADAR is endogenously expressed in a target cell in which the RNA sensor may be used.

There are millions of ADAR editing sites in the transcriptomes of humans and animals, only a small fraction of this editing occurs in coding mRNAs, altering protein properties. The vast majority are in non-coding regions, which may influence RNA splicing, microRNA and shRNA functions. Their most essential role though is to protect cells from innate immune response to self-generated dsRNAs while letting the immune system destroy viral dsRNAs during an infection.

B. readrRNA

The “readrRNA” refers to an RNA based molecule having a 5′ region and a 3′ region, where the readrRNA molecule comprises, consists of, or consists essentially of (i) a 5′ region comprising a sensor (ses) domain, the sensor domain comprising at least one ADAR-editable STOP codon; and (ii) an effector RNA (efRNA) region that is downstream and in-frame with the sensor domain. The dual-function readrRNA of the present disclosure permits recruitment of the ADAR deaminase to edit a specific site(s) in the readrRNA by formation of a dsRNA having a mismatch with target RNA expressed in a selected somatic cell. Upon ADAR-mediated removal of at least one stop codon from the readrRNA molecule, translation of a downstream operably linked effector protein encoded by the readrRNA occurs in the selected somatic cell. In the absence of target RNA in the selected somatic cell, the readrRNA remains inert in the cell.

Because the readrRNA detects or “senses” target RNA in the selected somatic cell, the readrRNA is an integral component of the system comprised by CellREADR (Cell access through RNA sensing by Endogenous ADAR), a programmable RNA sensing technology that leverages RNA editing mediated by ADAR (adenosine deaminase acting on RNA) for coupling the detection of cell-type defining RNAs with the translation of effector protein(s) in a somatic cell.

### C. Cell-type Defining RNAs

RNAs are the central and universal mediator of genetic information underlying the diversity of cell types and cell states, which together shape tissue organization and organismal function across species and life spans. Despite advances in RNA sequencing and massive accumulation of transcriptome datasets across life sciences, the dearth of technologies that leverage RNAs to observe and manipulate cell types remains a prohibitive bottleneck in biology and medicine. (Hongkui Zeng (2022) Cell 185:2739-2755).

Cell types are the product of evolution and they are the basic functional units of an organism. The entire repertoire of cell types in the brain and the body is built through a sequential and parallel series of spatially and temporally coordinated developmental events starting from a single fertilized egg, the zygote. This developmental program carries out a remarkable implementation plan that unravels the identities of all cell types which are encoded in the genome through evolution. Transcriptional and epigenetic regulatory programs are unfolded from the genome sequences and drive a cascading series of cell proliferation and differentiation processes, leading to the manifestation of diverse cellular phenotypes. (Hongkui Zeng (2022) Cell 185:2739-2755).

As used herein, a “cellular RNA” means an RNA that is present in a given cell, whether the RNA is endogenous to the cell (i.e., transcribed from a gene endogenous to the cell), or is present in the cell because it is transcribed from a gene that has been introduced into the cell, or is transcribed from a pathogen (such as a virus, bacteria, fungus or another micro-organism) that has infected the cell.

As used herein, a “cellular RNA of a cell” means an RNA that is present in a cell that, as a result of possessing specific characteristic, is identifiable because the RNA is known to be present in a cell having those specific characteristics.

As used herein, a “cell state-defining cellular RNA” refers to one or more RNA sequences present in a select cell or group of cells of interest, the presence of which identifies the state of a given cell, including but not limited to, a specified cell physiology, a specified development stage of a cell, a specified transformation of a cell, or activation state of a cell.

That is, the specific physiology of a cell is in large part determined by its expression of a unique repertoire of RNA transcripts. The unique repertoire of RNA transcripts is one means of identifying a specific cell or group of cells of interest, or identifying a specific activation state of a specific cell or group of cells of interest, or of identifying a specific developmental state of a specific cell or group of cells of interest, or identifying any one of numerous physiological states a cell or a specific cell or group of cells of interest. RNA expression profiles underlie arguably all phenotypic features of the cell at the time or state when the cell is characterized and is a one-time snapshot of the cell. A key point of distinction is whether the RNA expressed in a selected cell represent a particular cell state-a transient or dynamically responsive property of a cell to a context- or a cell type, as a cell type can exist in different states. Cell type-specific changes in RNA expression associated with different cell states may be seen during circadian cycles, variable metabolic states, development, aging, or under behavioral, pharmacological, or diseased conditions (Mayr et al., Development (2019) 146 (12): dev176727; Morris, S. A. (2019) Development 146, dev169748; (Hongkui Zeng (2022) Cell 185:2739-2755). A single-cell transcriptome is only a one-time snapshot of the cell. However, one can compare transcriptomes collected from different time points or different behavioral, physiological, or pathological states. The distinction between cell types and cell states is particularly challenging during development, as cells continually change their states, and at certain key time points, they may switch their cell type identities. However, although not absolute, it is reasonable to assume that transcriptomic changes tend to be more continuous during cell state transitions, while tending to be more abrupt or discrete when cells switch their types. (Hongkui Zeng (2022) Cell 185:2739-2755).

For a definition of Cell-type to be meaningful, it is ideally associated with what the cell type does. Thus, in addition to defining a cell type based on its Cell-type defining RNAs, a cell types is defined by linking its RNfA expression to anatomical and functional information. So far, it has been shown that transcriptomic types have excellent correspondence with their spatial distribution patterns. Since the spatial distribution pattern is defined during development, this suggests that transcriptomes may retain the developmental plan. ((Hongkui Zeng (2022) Cell 185:2739-2755).

Systematic single-cell transcriptomic, epigenomic and spatially resolved transcriptomic profiling with high temporal resolution, coupled with lineage tracing and other phenotypic characterization, holds tremendous potential to capture key sets of genes and genomic regulatory networks involved in these series of events and begin to resolve the extremely complex spatial and temporal transitions of cell types and states leading to the adult-stage repertoire of cell types (Allaway, K. C., Gabitto, M. I., Wapinski, O. et al. Genetic and epigenetic coordination of cortical interneuron development. Nature 597, 693-697 (2021). https://doi.org/10.1038/s41586-021-03933-1; Bandler, R.C., Vitali, I., Delgado, R. N. et al. Single-cell delineation of lineage and genetic identity in the mouse brain. Nature 601, 404-409 (2022). https://doi.org/10.1038/s41586-021-04237-0; Bhaduri, A., Sandoval-Espinosa, C., Otero-Garcia, M. et al. An atlas of cortical arealization identifies dynamic molecular signatures. Nature 598, 200-204 (2021). https://doi.org/10.1038/s41586-021-03910-8; Cao, Junyue, et al. “The single-cell transcriptional landscape of mammalian organogenesis.” Nature 566.7745 (2019): 496-502; Chen, Ao, et al. “Spatiotemporal transcriptomic atlas of mouse organogenesis using DNA nanoball-patterned arrays.” Cell 185.10 (2022): 1777-1792; Delgado, R.N., Allen, D. E., Keefe, M. G. et al. Individual human cortical progenitors can produce excitatory and inhibitory neurons. Nature 601, 397-403 (2022). https://doi.org/10.1038/s41586-021-04230-7; Di Bella, D.J., Habibi, E., Stickels, R. R. et al. Molecular logic of cellular diversification in the mouse cerebral cortex. Nature 595, 554-559 (2021). https://doi.org/10.1038/s41586-021-03670-5; Klingler, E., Tomasello, U., Prados, J. et al. Temporal controls over inter-areal cortical projection neuron fate diversity. Nature 599, 453-457 (2021). https://doi.org/10.1038/s41586-021-04048-3; La Manno, G., Siletti, K., Furlan, A. et al. Molecular architecture of the developing mouse brain. Nature 596, 92-96 (2021). https://doi.org/10.1038/s41586-021-03775-x; Romanov, R.A., Tretiakov, E. O., Kastriti, M. E. et al. Molecular design of hypothalamus development. Nature 582, 246-252 (2020). https://doi.org/10.1038/s41586-020-2266-0; Schmitz, M. T., Sandoval, K., Chen, C. P. et al. The development and evolution of inhibitory neurons in primate cerebrum. Nature 603, 871-877 (2022). https://doi.org/10.1038/s41586-022-04510-w; Sharma, N., Flaherty, K., Lezgiyeva, K. et al. The emergence of transcriptional identity in somatosensory neurons. Nature 577, 392-398 (2020). https://doi.org/10.1038/s41586-019-1900-1; Shekhar et al., 2022 elife 11, e73809; Tiklova et al., 2019 Nature Commun 10:581; Zhu, Ying, et al. “Spatiotemporal transcriptomic divergence across human and macaque brain development.” Science 362.6420 (2018): eaat8077.). ((Hongkui Zeng (2022) Cell 185:2739-2755)

### D. Cell Types

The inventors have demonstrated that increased IL-6 signaling in neurons in the area postrema (AP), a circumventricular structure in the hindbrain, drives cachexia in tumor-bearing mice while on the other hand, a reduction in IL-6 signaling in AP neurons of otherwise healthy mice causes overeating and increased blood glucose, suggesting that IL-6 normally conveys a satiety signal through AP neurons. As used herein, the term “area postrema neurons” or “AP neurons” refers to those neurons (or neuronal cells) found in the area postrema (AP) located in the hindbrain of the mammalian brain.

The cell readrRNA system as provided herein provides a system to simultaneously monitor cell type and states thereof, based on transcripts as well as spatially/morphologically by linking the targeting specific RNA transcripts with expression of an encoded effector molecule such as a fluorescent protein, the cell readrRNA system

As provided herein, the CellReadr system is based on Watson-Crick base-pairing and RNA editing, CellREADR 1) has inherent and absolute specificity to cellular RNA and cells defined by RNA expression; 2) easy to design, build, use, and disseminate (e.g., DNA vectors); 3) infinitely scalable for targeting all RNA-defined cell types in any tissue; libraries of “cell armamentarium” 4) generalizable to most animal species including human; 5) comprehensive for most cell types and tissues and 6) general across animal species, 7) applicable to human biology and medicine, 8) programmable to achieve intersectional targeting of cells defined by two or more RNAs and multiplexed targeting and manipulation of several cell types in the same tissue.

### E. CellREADR Technology

As used herein, “CellREADR” stands for “Cell access through RNA sensing by Endogenous ADAR [adenosine deaminase acting on RNA]”, and it is designed as a single, modular Readr RNA molecule, consisting of a 5′ sensor-edit-switch region (sesRNA) and a 3′ effector protein (or protein fragment) coding region (ef RNA), separated by a link sequence coding for a self-cleaving peptide T2A and an editing mechanism ubiquitous to all animal cells, such as by an ADAR-editable STOP codon. CellREADR provides a mechanism for detecting the presence of cellular RNAs and switching on the translation of effector proteins to monitor and manipulate physiology, functions and/or structure of a cell type.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

In some embodiments, a polypeptide, can comprise one or more amino acid substitutions or modifications. In some embodiments, the substitutions and/or modifications can prevent or reduce proteolytic degradation and/or prolong half-life of the polypeptide in a subject. In some embodiments, a polypeptide can be modified by conjugating or fusing it to other polypeptide or polypeptide domains such as, by way of non-limiting example, transferrin (WO06096515A2), albumin (Yeh, Patrice, et al. “Design of yeast-secreted albumin derivatives for human therapy: biological and antiviral properties of a serum albumin-CD4 genetic conjugate.” Proceedings of the National Academy of Sciences 89.5 (1992): 1904-1908), growth hormone (US2003104578AA); cellulose (Levy, Ilan, and Oded Shoseyov. “Cellulose-binding domains: biotechnological applications.” Biotechnology advances 20.3-4 (2002): 191-213.); and/or Fc fragments (Ashkenazi, Avi, and Steven M. Chamow. “Immunoadhesins as research tools and therapeutic agents.” Current opinion in immunology 9,2 (1997): 195-200). The references in the foregoing paragraph are incorporated by reference herein in their entireties.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

That is, the core of the CellREADR technology is the readrRNA which is an RNA sequence specific molecular sensor-switch operably linked to an effector molecule. That is a single modular readrRNA comprises a 5′-prime sense-edit-switch domain (sesRNA) and a 3′-prime effector domain (efRNA). The target specificity of sesRNA is due to its interaction with complementary sequences on target mRNA. The degree of complementarity determines whether there is ADAR-mediated editing of the sesRNA. A sesRNA which is fully complementary to the target RNA induces ADAR-mediated editing of the sesRNA at the ADAR editable stop codon.

As used herein, a “CellREADR system” includes the following components: (i) a sensor RNA domain which comprises a consecutive set of nucleotides that is complementary to a portion of a selected cellular RNA, (ii) an effector RNA (efRNA) domain encoding an effector protein, the efRNA domain being downstream of and in-frame with the sensor RNA domain, (iii) an ADAR-editable STOP codon that lies within the sensor RNA domain or lies between the sensor and effector RNA domains, and (iv) a second protein coding nucleic acid or a gene optionally including gene control elements, where (iv) that may or may not be physically linked to the sensor RNA and effector RNA domains. A CellREADR System may include an exogenous gene (DNA or RNA) not physically linked to the readrRNA (e.g., on a separate vector). A CellREADR System may include a cell that contains the readrRNA nucleic acid, a nucleic acid encoding a second protein, the cell being used for delivery to a multicellular organism, a plant, an animal, a mammal or a primate, a human or mouse.

In the context of polypeptides and molecules as provided herein, the terms “fusion”, “fused,” “combination,” and “linked,” are used interchangeably herein. These terms refer to the joining together of two more protein components, by whatever means including chemical conjugation or recombinant means. For example, a promoter or enhancer is operably linked to a coding sequence if it affects the transcription of the sequence. Generally, “operably linked” means that the DNA sequences being linked are contiguous, and in reading phase or in-frame. As used herein, the term “in-frame” or “in frame” refers to the joining of two or more open reading frames (ORFs) to form a continuous longer ORF, in a manner that maintains the correct reading frame of the original ORFs. Thus, the resulting recombinant molecule is a single protein containing two or more segments that correspond to polypeptides encoded by the original ORFs (which segments are not normally so joined in nature).

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence. Further they need not be physically linked.

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Heterologous” means derived from a genotypically distinct entity from the rest of the entity to which it is being compared. For example, a glycine rich sequence removed from its native coding sequence and operatively linked to a coding sequence other than the native sequence is a heterologous glycine rich sequence. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” or “gene fragment” are used interchangeably herein. They refer to a polynucleotide containing at least one open reading frame that is capable of encoding a particular protein after being transcribed and translated. The term “gene” includes not only an open reading frame but also at least a promoter operatively associated with the open reading frame so as to initiate transcription of the open reading frame in the presence of appropriate transcription factors. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

“Homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity to those sequences.

The phrase “complementary to” “complement of a polynucleotide” denotes a polynucleotide nucleic acid molecule having a complementary having a base sequence in a 5′ to 3′ or 3′ to 5′ orientation that base pairs with a nucleic acid having a base sequence of the reverse orientation. As described herein, a sensor RNA is complementary to a specified target RNA, with the exception of an obligatory mismatched codon, (preferably AUG in the sensor RNA).

The phrase “portion that is complementary to a cellular RNA” in the context of a sensor domain refers to consecutive nucleotides of a sensor nucleic acid domain that are able to base pair with corresponding consecutive nucleotides of a cellular RNA.

The phrase “portion that is complementary to a messenger RNA (mRNA)” in the context of a sensor domain refers to consecutive nucleotides of a sensor nucleic acid domain that are able to base pair with corresponding consecutive nucleotides of an mRNA.

F. Sensor Domain of Modular readrRNA

The sensor domain (sesRNA) comprises a set of nucleotides that are complementary to and able to detect a specific cell type through sequence-specific base pairing with an RNA present in the specific cell type. The sensor domain may comprise any number of nucleotides. In some embodiments, the sensor domain comprises at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000. In some embodiments, the sensor domain comprises a range of about 100 to about 900 nucleotides. In another embodiment, the sensor domain comprises a range of about 200 nucleotides to about 600 nucleotides. Preferably, the sensor domain (sesRNA) contains ˜250 nucleotides to ˜575 nucleotides, complementary to and thus can detect a specific cell type RNA through base pairing.

In some embodiments, the sensor domain also includes one or more ADAR-editable STOP codons that act as a translation switch (termed herein as the sense-edit-switch RNA (sesRNA)). The sensor domain thus functions as a sense-edit-switch RNA (sesRNA). The sensor RNA comprises a nucleotide sequence that is complementary to a cellular RNA.

The modular readrRNA molecule also may include a sequence coding for a self-cleaving 2A peptide. In such embodiments, the 2A peptide is positioned in between domains, preferably between the sensor domain and the effector RNA region and/or between additional domains/regions that may be present in the readrRNA molecule or CellREADER system, as discussed further herein.

G. In-frame effector RNA (efRNA) coding region of Modular readrRNA

Downstream to the sensor domain and, optionally separated by the sequence coding for the self-cleaving 2A peptide, is an in-frame effector RNA (efRNA) coding region. The readrRNAs can be generated from conventional DNA expression vectors. These vectors consist of a promoter, DNA cassettes coding for sesRNA and efRNA, and 3′ untranslated regions, which can be assembled by routine DNA synthesis and molecular cloning. In one embodiment, the sesRNA coding cassette may be --200-300 base pairs, and the effector gene cassette may be --1-2 kilo base pairs. These expression vectors can be readily packaged into various viral particles. readrRNAs can also be generated by direct single-strand oligonucleotide synthesis, with incorporation of chemically modified nucleotides if necessary.

H. Modular readrRNA

The term “modular” when used in the context of the phrase “Modular readrRNA Molecule” refers a recombinant readrRNA molecule comprising nucleic acid sequences (preferably RNA sequences) encoding protein domains designed at the nucleic acid level, preferably at the RNA level, where the different protein domains can be assembled in the recombinant readrRNA molecule in the desired order with a specified number of repeats (including 0).

Accordingly, one aspect of the present disclosure provides a modular readrRNA molecule comprising, consisting of, or consisting essentially of (i) a 5′ region comprising a sensor domain, the sensor domain comprising at least one ADAR-editable STOP codon; and (ii) an effector RNA (efRNA) region that is downstream and in-frame with said sensor domain. The term “modular” when used in the context of the phrase “Modular readrRNA Molecule” refers a recombinant readrRNA molecule comprising a combination of a much smaller number of linked structural unit, where each structural unit encodes an independently functioning protein molecule.

The modular design of the readrRNA molecule, in which different protein encoding domains are designed at the RNA level and which are assembled in the recombinant readrRNA molecule in a desired order with a specified number of repeats design, enables the production of readrRNA molecules with diverse properties. The translation machinery also has high fidelity so that the desired readrRNA molecule will have the specified amino acid sequence.

In general, a readrRNA molecule is composed of modular domains that confer specific functions, including but not limited to facilitation of interactions between cells, sensing environmental stimuli, effecting a response to environmental stimuli, including effecting spatiotemporal input/output in a biological system.

I. Sensor Domain of Modular readrRNA

The sensor domain (sesRNA) comprises a set of nucleotides that are complementary to and able to detect a specific cell type through sequence-specific base pairing with an RNA present in the specific cell type. The sensor domain may comprise any number of nucleotides. In some embodiments, the sensor domain comprises at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000. In some embodiments, the sensor domain comprises a range of about 100 to about 900 nucleotides. In another embodiment, the sensor domain comprises a range of about 200 nucleotides to about 600 nucleotides. Preferably, the sensor domain (sesRNA) contains ˜250 nucleotides to ˜575 nucleotides, complementary to and thus can detect a specific cell type RNA through base pairing.

In some embodiments, the sensor domain also includes one or more ADAR-editable STOP codons that act as a translation switch (termed herein as the sense-edit-switch RNA (sesRNA)). The sensor domain thus functions as a sense-edit-switch RNA (sesRNA). The sensor RNA comprises a nucleotide sequence that is complementary to a cellular RNA.

As used herein, a “translation switch” is a component of a readrRNA molecule comprising an ADAR-editable STOP codon component which, upon binding by upstream sensor RNA to complementary target RNA to form a double stranded RNA structure, results in subsequent ADAR mediated editing of the AUG stop codon, resulting in the translation of the downstream RNA that encodes for an effector protein.

The modular readrRNA molecule also may include a sequence coding for a self-cleaving 2A peptide. In such embodiments, the 2A peptide is positioned in between domains, preferably between the sensor domain and the effector RNA region and/or between additional domains/regions that may be present in the readrRNA molecule or CellREADER system, as discussed further herein.

J. In-Frame Effector RNA (efRNA) Coding Region of Modular readrRNA

Downstream to the sensor domain and, optionally separated by the sequence coding for the self-cleaving 2A peptide, is an in-frame effector RNA (efRNA) coding region. The effector RNA (efRNA) may code for an effector protein of interest, such as a label allowing visualization of the labeled cell. The effector RNA (efRNA) may code for an effector protein that changes the physiology of a cell. For example, in the case of a treating a disease caused by a mutation in single gene, the encoded effector protein can be a corrected copy of the mutated gene. In the case of treating a disease by providing a certain protein, be it endogenous or exogenous to the organism, the protein can be encoded in the effector region.

Selection of a given efRNA is dependent on the desired use of the readrRNA (e.g., treatment of a disease, study of a protein/pathway, etc.) of the readrRNA molecule and can be readily determined by one skilled in the art. For example, the effector module of CellREADR (efRNA) can be built to manipulate cells in multiple ways, including enhance activity and function, suppress activity and function, rescue a mutant cell function by reintroducing an intact version of the deleted or mutated protein, alter and edit activity and function, reprogram cell identity, fate, and function, kill and delete a cell type, increase or decrease the production of cell numbers of a type, and cell type-specific genomic editing and gene regulation.

In cells expressing the target RNA, the sesRNA forms dsRNA, which recruits endogenous ADAR enzyme. At the STOP codon, A to I editing converts the STOP to a TI (G) G tryptophan codon, switching on translation of the efRNA, and generation of effector proteins. The resulting fusion protein comprising an N-terminal peptide, T2A and C-terminal effector, which then self-cleaves through T2A, releasing the functional effector protein. In cells that do not express the target RNA, readrRNAs remain inert.

The efRNA-encoded protein may comprise any protein involved in, or that is able to influence cell replication, gene expression, and/or transcription/translation. Suitable examples include, but are not limited to, a transcriptional activator, a transcriptional inhibitor, and a DNA recombinase, and the like.

An effector protein includes, but is not limited to, A) an enzyme, for example, proteases, phosphatases, glycosylases, acetylases, or lipases, b) a protein that mimics a function of a host cell protein, c) a transcription factor, d) a protein partner that facilitates protein-protein interaction, d) a protein that alters host cell structure and function, for example by facilitating infection (a virulence factors or a toxin) and/or by triggering a defense response, and/or promoting morphogenesis (Cachat, E., Liu, W., Hohenstein, P. et al. A library of mammalian effector modules for synthetic morphology. J Biol Eng 8, 26 (2014). https://doi.org/10. 11 86/1 754-1611-8-26).

For instance, exemplary effector proteins are listed in the Table 2 below.

TABLE 2

| | Examples on Cellular | |
|---|---|---|
| Disease | Pathway | Payload Examples |
| Obesity | Increase activity of Gfal- | IL6a; sodium channel (e.g., |
| | expressing (Gfral⁺) AP | mNaChBac); a mutant AMPA |
| | neurons | receptor (e.g. GluA2-L483Y- |
| | | R845A); GluA4 |
| Cancer | Decrease activity of Gfal- | IL6ra shRNA; Tetanus Toxin |
| Cachexia | expressing (Gfral+) AP | Light Chain (TeLC); a |
| | neurons | dominant negative Ras; a |
| | | dominant negative STAT3; |
| | | GluA4 C-tail |

Thus, effector functions can influence activities of the innate immune cell response, including phagocytosis, secretion of cytokines, trafficking or promoting function, migration, survival, expression of surface receptors, and proliferation of immune cells.

Thus, the encoded effector molecule can be a transactivator or a transrepressor, stimulating or suppressing, respectively, expression of a gene of interest by binding to the promoter/enhance region of the gene of interest, be it an endogenous gene, or an exogenous gene administered as part of the cell rear system.

A transcriptional activator is a protein or small molecule that binds to one or more specific regulatory sequences in DNA (or RNA in the case of a retrovirus) and stimulates transcription of one or more nearby genes. Most activators enhance RNA polymerase binding (formation of the closed complex) or the transition to the open complex required for initiation of transcription. Most activators interact directly with a subunit of RNA polymerase.

A transcriptional repressor is sequence-specific DNA binding proteins generally thought to function by recruiting corepressor complexes, which contain multiple proteins including histone modifying enzymes.

As used herein, “modulated” means regulated in the sense of activated or inhibited.

As used herein, a pathogen comprises an organism that causes disease in human beings, A pathogen includes but is not limited to a bacterium, a virus, a parasite, an insect, an algae, a prion and a fungus).

As used herein, the term “stop codon” refers to a sequence of three nucleotides (a trinucleotide) in DNA or messenger RNA (mRNA) that signals a halt to protein synthesis in the cell.

A “codon” in a messenger RNA corresponds to a nucleotide triplet that encodes an amino acid. Consecutive codons in an RNA are translatable to a protein. In nature, a stop codon is located in the 3′ terminal end of the coding region(s) of a mRNA and signals the termination of translation by binding release factors, which binding causes the ribosomal subunits to disassociate and thereby to release the amino acid chain. There are 64 different trinucleotide codons: 61 specify amino acids and 3 are stop codons (i.e., UAA, UAG and UGA in RNA and TAA, TAG and TGA in DNA).

As used herein, an “editable stop codon” refers to a stop codon that is editable by a cell from a stop codon to a translatable codon. Thus, in RNA, an editable stop codon which is a UAA, a UAG or a UGA is editable by a cell to UII, UIG, or UGI. An editable stop codon functions as a translation switch for any codons downstream of the editable stop codon. Editing of a stop codon occurs in cells in which an endogenous ADAR enzyme is present.

“Editing” of a stop codon occurs when a sensory RNA containing an editable stop codon forms dsRNA with a target RNA, thereby recruiting endogenous ADAR enzyme. ADAR acts at the STOP codon, performs A to I editing and thus converts for example a UAG STOP to a UIG (tryptophan) codon, which permits translation of downstream codons.

The term “ADAR” is a disambiguation that stands for adenosine deaminase acting on RNA. ADAR enzymes bind to double-stranded RNA (dsRNA) and convert adenosine to inosine (hypoxanthine) by deamination. ADAR proteins act post-transcriptionally, changing the nucleotide content of RNA. The conversion from adenosine to inosine (A to I) in the RNA disrupts the normal A: U pairing, destabilizing the RNA. Inosine is structurally similar to guanine (G) which leads to inosine to cytosine (I: C) binding. Inosine typically mimics guanosine during translation but can also bind to uracil, cytosine, and adenosine, though it is not favored.

As used herein, the term “specific binding” refers to a chemical interaction between two molecules, compounds, cells and/or particles wherein the first entity binds to the second, target entity with greater specificity and affinity than it binds to a third entity which is a nontarget. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

As used herein, “readrRNA” refers to a molecule having a 5′ region and a 3′ region, where the readrRNA molecule comprises, consists of, or consists essentially of (i) a 5′ region comprising a sensor (ses) domain, the sensor domain comprising at least one ADAR-editable STOP codon; and (ii) an effector RNA (efRNA) region that is downstream and in-frame with the sensor domain.

In a readrRNA, an ADAR-editable stop codon is located within the sensor RNA and upstream of the in-frame effector coding region.

s used herein, an “ADAR-editable STOP codon” refers to a stop codon that is editable in a cell by ADAR. Schneider, M. F., Wettengel, J., Hoffmann, P. C., & Stafforst, T. (2014). Optimal guide RNAs for re-directing deaminase activity of hADAR1 and hADAR2 in trans. Nucleic acids research, 42 (10), e87. https://doi.org/10.1093/nar/gku272

As used herein, a “sensor domain” refers to a consecutive set of nucleotides that form a portion of a readrRNA, where the sensor domain also includes at least one editable stop codon and a downstream effector domain. A sensor domain contains consecutive nucleotides that are complementary to an RNA of a specific cell type through sequence-specific base pairing. A sensor domain may comprise any number of nucleotides, comprising at least 10 nucleotides to at least 1000 nucleotides or more. In some embodiments, the sensor domain comprises, consists essentially of or consists of about 100 to about 900 nucleotides. In another embodiment, the sensor domain comprises, consists essentially of or consists of a range of about 200 nucleotides to about 300 nucleotides. A sensor domain may be 10, 25, 50, 75, 100, 150, 200, 250, 300, 350, 400, 450, 500, 550, 600, 650, 700, 750, 800, 850, 900, 950, 1000 or more consecutive nucleotides in length.

The at least one editable stop codon(s) in a sensor domain is/are located anywhere within the sensor domain of the readrRNA. A sensor domain will have a 5′ to 3′ orientation in the readrRNA molecule and includes an upstream (5)′ portion and a downstream (3)′ portion. An editable stop codon may be located in the sensor domain upstream portion or the sensor domain downstream portion. An editable stop codon may be located in the upstream portion of the sensor domain closer to the middle of the sensor domain or an editable stop codon may be located in the downstream portion of the sensor domain closer to the downstream end of sensor domain. For example, if the sensor domain is 600 nucleotides in length and divided spatially into halves, with the first nucleotide representing the 5′ end of the sensor domain, the 300th nucleotide representing middle of the sensor domain, and the last (600th) nucleotide of the sensor domain representing the 3′ end of the sensor domain, an editable stop codon may be located in the upstream portion of the downstream portion or closer to the middle of the sensor domain. If a sensor domain having the same approximate length is divided into quarters, an editable stop codon may be located in the first quarter portion (nucleotides 1-250) of the sensor domain, the second quarter portion of the sensor domain (nucleotides 150-300), the third quarter portion of the sensor domain (nucleotides 300-450), or the fourth quarter portion of the sensor domain (nucleotides 450-600). Thus, for a given length of a sensor domain, an editable stop codon may be located in a selected portion of the sensor domain. Generally, an editable stop codon may be located in the downstream half of a sensor domain, or the downstream quarter of a sensor domain. A selected portion of the sensor domain containing an editable stop codon may be within 10-50 nucleotides of the 3′ end of the sensor domain.

As used herein, an “effector RNA (efRNA)” is RNA that is translatable and encodes an effector protein. As used herein, an “effector RNA (efRNA) region” refers to a portion of a readrRNA comprising an effector RNA that is downstream and in-frame with a sensor domain.

An “effector protein” is a protein encoded by an effector RNA domain and that has an effect on a cell in which it is expressed. An effector protein is translated from an effector RNA in a cell and therefore an effector protein, like the RNA encoding it, is introduced into a cell that may or may not contain the same endogenous protein. An effector protein is a protein having an effect on the cell in which it is translated or, if secreted from the cell, on surrounding cells. No limiting examples of effector proteins include: an enzyme, a detectable protein, a cytokine, a toxin, a polymerase, a transcription or translation factor, a tumor suppressor, a neuronal activator or inhibitor, an apopotic protein or a physiological factor.

The effector RNA (efRNA) may code for an effector protein of interest. Selection of a desired effector protein is well within the skill of one of ordinary skill in the art and is dependent on the context of the desired use of the readrRNA. For example, if it is desired to treat a given disease, an effector protein may be selected based on its having an inhibitor effect on cells that are critical to establishing and/or prolonging the disease. For example, the effector module of CellREADR (efRNA) can be built to manipulate cells in multiple ways, including enhance activity and function, suppress activity and function, rescue a mutant cell function by re-introducing an intact version of the deleted or mutated protein, alter and edit activity and function, reprogram cell identity, fate, and function, kill and delete a cell type, increase or decrease the production of cell numbers of a type, and cell type-specific genomic editing and gene regulation.

### K. Reporter Gene of CellREADR System

Another aspect of the present disclosure provides a CellREADR system, the CellREADR system comprising at least two components, a first component comprising a modular readrRNA molecule as described herein and optionally an additional component(s) comprising a response gene operably linked (though in this embodiment, not physically linked) to the efRNA-encoded protein (e.g., transcriptional regulator, e.g., API and SPI.) of the readrRNA molecule. This allows the readrRNA molecule to activate, increase, decrease or repress transcription of another protein encoded on a physically separate, exogenously added nucleic acid molecule, such as caspase molecule which results in cell death.

### L. ADAR Mediated Programmability and Intersectional Targeting

The sensor and effector modules are combinatorial and easily programmable, which allows to manipulate each cell type in multiple ways and to simultaneously manipulate multiple cell types in a tissue, each in a specific and coordinated way. Such intersectional targeting provides for the specific targeting to a cell type (e.g., a neuronal cell) or cell state (e.g., a cancer cell) that are defined by two or more RNA biomarkers. “Biomarker” or “Marker” in the context of the present invention refers to an expression product, e.g., nucleic acid or polypeptide which is differentially present in a sample taken from subjects having diabetes or cancer, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

As provided above, in some embodiments the modular readrRNA molecule comprises, consists of, or consists essentially of a 5′ sensor-edit-switch region (sesRNA) and a 3′ effector coding region (efRNA), separated by an optional link sequence coding for a self-cleaving peptide 2A. In some embodiments, the sesRNA contains about 200 to about 600 nucleotides, complementary to and thus can detect a specific cell type RNA through base pairing while also comprising one or more ADAR-editable STOP codons that acts as a translation switch and wherein downstream is an in-frame effector coding region to generate various effector proteins of interest.

In other embodiments, the 5′ region comprises sensor domains comprising a stretch of consecutive nucleotides of two or more joint sensor domains that are complementary to a corresponding stretch of consecutive nucleotides of two or more cellular RNAs, respectively, of a cell of the mammalian central nervous system, wherein said sensor domain comprises two or more stop codons editable by ADAR; and the 3′ region comprises a domain encoding a protein, wherein said protein coding domain is downstream of and in-frame with said sensor domains, wherein, upon introduction of said modular RNA into said cell of said comprising an Adar enzyme, said stretch of consecutive nucleotides of said sensor domains and said corresponding nucleotide stretch of said cellular RNA form an RNA duplex comprising said stop codons, wherein said stop codons comprised in said RNA duplex is edited by ADAR in said cell, thereby to permit translation of said protein.

In cells expressing the target RNA, the sesRNA forms dsRNA with the target RNA, which recruits endogenous ADAR enzyme. At the STOP codon, A to I editing converts the STOP to a TI (G) G tryptophan codon, switching on translation of the efRNA, and generation of effector proteins. The resulting fusion protein comprising an N-terminal peptide, 2A and C-terminal effector, which then self-cleaves through 2A, releasing the functional effector protein. Importantly, in cells that do not express the target RNA, the readrRNAs remain inert. As such, the modular readrRNA molecules can thus be deployed as a single RNA molecular and can fit easily into viral vector (e.g., an AAV vector), as ADAR is cell endogenous.

In some situations, the ADAR protein(s) are not highly expressed, or in some cases absent, in the cell. In such cases, the present disclosure provides for the addition of the ADAR protein (e.g., the ADAR2) to be included within the modular readrRNA molecule and/or added to the system via a separate vector. The most fundament feature of CellREADR is that it is entirely RNA sequence based and operates through Watson-crick base pairing which confers numerous highly desirable properties, including, but not limited to, (i) inherent & absolute specificity to cellular RNAs; (ii) easy to design, build, use, and share (DNA vectors); (iii) infinitely scalable libraries of “cell armamentarium”; (iii) comprehensive for most cell types and tissues; (iv) general across animal species; and (v) human biology and medicine. Thus, comprehensive and combinatorial CellREADR sensor-effector libraries can be built for identifying, characterizing and manipulating cell types across organ systems and animal species.

The programmability of the modular readrRNA molecules provided herein confers additional power. Accordingly, another embodiment of the present disclosure provides for the programmability the modular readrRNA molecules and/or intersection targeting using the modular readrRNA molecules as provided herein.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell is typically still referred to as “engineered” even though the actual manipulation was performed on a prior entity.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

M. Modular readrRNA Molecules as Provided Herein.

First, two or more RNA sensors can be designed to detect two or more separate cellular RNAs to achieve intersectional targeting of two or more specific cell types. Second, the same RNA sensor can be linked to different effectors to label, record, and manipulate the same cell type. Third, a cohort of multiple RNA sensors can be designed to target several cell types in the same tissue, each expressing a different effector, to coordinately module tissue function. Fourth, RNA sensors can be designed to detect different threshold levels of a target RNA to monitor and manipulate different cell states defined by the RNA levels.

In one embodiment, the present disclosure provides for two sensors that are designed to detect two separate cellular RNAs to achieve intersectional targeting of more specific cell types. In one aspect, each sensor module has at least one stop codon, and only when both are removed can the effector molecule be expressed. In one embodiment, each sensor comprises at least one STOP codon. In another embodiment, the same sensor can be used to expression different effectors to label, record, and manipulate the same cell type.

In yet another embodiment, a plurality of sensors is designed to target several cell types in the same tissue, each expressing a different effector, to thereby coordinate module tissue function.

Thus, the RNA sensing domain has the capacity to detect any cellular RNA and thus the ability to access any RNA-defined cell types and cell states in any human tissues. The effector domain has the capacity to encode any protein and thus the ability to monitor, manipulate, and edits many cellular properties.

### N. Generality of Targeting Cell Types and Cell States

The RNA sensor domain can detect RNA markers that define cell types and cell states. Recent advances in single cell RNA sequencing are generating massive datasets in all human and animal tissues. Several major efforts are driving the progress, including the Human Cell Atlas project (world wide web at humancellatlas.org); the NIH Human Biomolecular atlas program at (commonfund.nih.gov/hubmap); the BRAIN Initiative Cell Census Network (biccn.org/); and the Allen Brain Cell Atlas (portal.brain-map.org/).

All the single cell transcriptome datasets are publicly accessible. RNA markers will be identified for most if not all major human cell types. Furthermore, RNA markers will be identified for many diseased cell states. All these RNA markers can be used by CellREADR to target cell types and cell states. Some of these markers are listed in Table 3.

TABLE 3

| Cell types/Tissue | Genes |
|---|---|
| Retina | RHO |
| Heart | NPPA |
| Smooth muscle | MYH1 |
| Adrenal gland | CYP1 1B1 |
| Parathyroid gland | GCM2 |
| thyroid gland | TG |
| Pituitary gland | TSHB |
| Lung | SFTPA1 |
| Bone marrow | CTSG |
| Lymphoid | CD1B |
| Liver | ALB |
| Gallbladder | FGF19 |
| Testis | LELP1 |
| Epididymis | DEFB106A |
| Prostate | TGM4 |
| Seminal Vesicle | SEMG2 |
| Adipose tissue | GYG2 |
| Inhibitory Neuron | VGAT |
| Excitatory Neuron | CAMK.2 |
| Neuron | NEUN |

The term “cellular RNA” refers to a nucleic acid in a cell composed of nucleotides that are substantially ribonucleotides but may include deoxyribonucleotides. Types of cellular RNAs include but are not limited to mRNA, rRNA, tRNA, and microRNA. A cellular RNA will have a length sufficient to form a nucleic acid duplex with a sensor RNA containing a mismatch that attracts ADAR to edit and repair the mismatch. Therefore, a cellular RNA will be at least 10 residues in length, and may be 15, 20, 25, 30, 35, 40, 45, 50, 75, 100, 125, 150, 175, 200, 225, 250, 275, 300, 325, 350, 375, 400, 425, 450, 475, 500, 600, 700, 800, 900, 1000 nucleotides in length or longer.

### O. Delivery

CellREADR can be deployed as a single RNA molecular, as ADAR is cell endogenous. And can fit easily into AAV viral vector in <4.7 Kbs. In practice, the entire readrRNA is several kilobases, depending on what specific sensors and effectors are incorporated into the molecule, and thus is deliverable to cells through a delivery system. In cells expressing the target RNA, sesRNA forms a dsRNA with the target, which recruits ADARs to assemble an editing complex. At the editable STOP codon, ADARs convert A to I, which pairs with the opposing C in the target RNA This A->G substitution converts a TAG STOP codon to a TI (G) G tryptophan codon, switching on translation of efRNA The in-frame translation generates a fusion protein comprising an N-terminal peptide, 2A (if being used), and C-terminal effector, which then self-cleaves through 2A and releases the functional effector protein. readrRNA remains inert in cells that do not express the target RNA.

Through this disclosure and the knowledge in the art, the modular readrRNA molecules provided herein, or any components thereof, nucleic acid molecules thereof, and/or nucleic acid molecules encoding or providing components thereof, as well as any CellREADR systems as provided herein, can be delivered by various delivery systems.

As used herein, an “delivery system” refers to a system comprising a vehicle for administering a modular readrRNA molecule and/or CellREADR system, where the vehicle includes but is not limited to a nanoparticle, a liposome, a vector, an exosome, a microvesicle, a gene-gun, a SEND system, and combinations thereof.

Examples of such delivery systems include, but are not limited to, DNA or RNA transfection method: chemical reagents (PEI, lipofectamine, calcium phosphate etc.) or electroporation, DNA expression vectors can be packaged into Liposome nanoparticles. readrRNAs can be transcribed or synthesized in vitro and packaged into Liposome nanoparticles, nanoparticles, liposomes, recombinant viral vectors (Viral vectors: Adena-associated virus (AAV), lenti-virus, and vesicular stomatitis virus are preferred viral vehicles), electroporation exosomes, microvesicles, gene-guns, the Selective Endogenous eNcapsidation for cellular Delivery (SEND) system, (an mRNA delivery system comprising humanized virus-like particles (VLPs) based on retroelements present in the human genome, (Segel M, et al. Mammalian retrovirus-like protein PEG10 packages its own mRNA and can be pseudotyped for mRNA delivery. Science. 2021; 373:882-889. doi: 10. 1 126/science.abg6155), combinations thereof, and the like.

As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Exemplary nanoparticles include lipid nanoparticles or ferritin nanoparticles. Lipid nanoparticles can comprise multiple components, including, e.g., ionizable lipids (such as MC3, DLin-MC3-DMA, ALC-0315, or SM-102), pegylated lipids (such as PEG2000-C-DMG, PEG2000-DMG, ALC-0159), phospholipids (such as DSPC), and cholesterol.

Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

The modular readrRNA molecules and/or any of the RNAs (e.g., sesRNA, efRNA, etc.) and/or any accessory proteins and/or CellREADR systems can be delivered using suitable vectors, e.g., plasmids or recombinant viral vectors, such as adeno-associated virus (AAV), adenovirus, retrovirus, lentivirus, herpes viral vector, vesicular stomatitis virus, and other viral vectors or combinations thereof. The proteins, e.g., sesRNA, efRNA, efRNA response genes, protein encoding or non-encoding RNAs (e.g., sgRHA, shRNA, etc.), Cell READR systems, etc., can be packaged into one or more vectors, e.g., plasmids or viral vectors. For example, in some embodiments, a second expression vector that comprises an efRNA response gene operably linked to the efRNA-encoded protein is co-delivered with the readrRNA molecule and/or CellREADR system, wherein upon successful translation of the modular readrRNA molecule and effector RNA results in successful binding and activation of the reporter product. In some embodiments, the efRNA response gene comprises a reporter gene (e.g., reporter genes including, but not limited to, GFP, mRuby, mCherry, ChR2, DTA, Gcamp, TK, interferon, etc.). In other embodiments, the efRNA response gene comprises a secondary effector gene.

For bacterial applications, if applicable, the nucleic acids encoding any of the components of the modular readrRNA molecule systems described herein can be delivered to the bacteria using a phage. Exemplary phages include, but are not limited to, T4 phage, u, q11, phage, TS phage, T7 phage, T3 phage, q29, M13, MS2, QB, and oX174. In such embodiments, the addition of exogenous ADAR may be required.

In some embodiments, the vectors, e.g., plasmids or recombinant viral vectors, are delivered to the tissue of interest by, e.g., intramuscular injection, intravenous administration, transdermal administration, intranasal administration, oral administration, or mucosal administration. Such delivery may be either via a single dose, or multiple doses.

One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine. In some embodiments, the recombinant viral vector comprises an adenovirus vector which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses.

In some embodiments, the delivery is via a recombinant adeno-associated virus (rAAV) vector. For example, in some embodiments, a modified AAV vector may be used for delivery. Modified AAV vectors can be based on one or more of several capsid types, including AAV1, AV2, AAV5, AAV6, AAV8, AAV 8.2. AAV9, AAV rhI0, modified AAV vectors (e.g., modified AAV2, modified AAV3, modified AAV6) and pseudotyped AAV (e.g., AAV2/8, AAV2/5 and AAV2/6), AAV-PHP.eB and any variants thereof, AAV-PHP.S and any variants thereof, AAV-PHP. V1 and any variants thereof, and the like. Exemplary AAV vectors and techniques that may be used to produce rAAV particles are known in the art.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include: (i) a promoter; (ii) a sequence encoding a modular readrRNA molecule and/or CellREADR system as provided herein, each operably linked to a promoter (e.g., the same promoter or a different promoter); (iii) a selectable marker; (iv) an origin of replication; and (v) a transcription terminator downstream of and operably linked to (ii). The plasmids can also encode other RNA components, but one or more of these may instead be encoded on different vectors. The frequency of administration is within the ambit of the medical or veterinary practitioner (e.g., physician, veterinarian), or a person skilled in the art.

In those cells/systems (e.g., bacterial cells and certain plant cells) where the ADAR protein is not expressed, or is expressed at low levels, plasmids (either alone or those expressing a modular readrRNA molecule and/or a CellREADR system as provided herein), may further comprise a sequence encoding an ADAR gene (e.g., Adar1, Adar2, etc.). Exogenous ADARs or engineered ADARs (i.e. with improved functionality) may increase the efficiency of CellREADR system. Exogenous ADAR can be delivered into animal (or plant) cells in the following ways that include, but are not limited to:
1) ADAR1 or ADAR2 is delivered with a separate construct/DNA vector (from the Readr construct) with CMV, CAG promoter, by transfection or virus infection (AAV, lentiviruses, etc).
2) ADAR1 or ADAR2 is placed in front of SesRNA sequence in the same CellReadr construct/DAN vector, and delivered into cells by transfection or virus infection.
3) ADAR1 or ADAR2 mRNAs are delivered into cells by LNPs, with the CellReadr DNA vector or RNA
4) ADAR1 or ADAR2 proteins are delivered into cells by LNPs or VLP (viral like particles), with the CellReadr DNA vector or RNA.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety. In some embodiments, the delivery is via nanoparticles or exosomes. For example, exosomes have been shown to be particularly useful in delivery RNA.

Further means of introducing one or more components of the modular readrRNA molecule systems as provided herein to the cell is by using cell penetrating peptides (CPP). In some embodiments, a cell penetrating peptide is linked to the modular readrRNA molecule.

In some embodiments, the modular readrRNA molecule and/or any components thereof are coupled to one or more CPPs to effectively transport them inside cells (e.g., plant protoplasts). In some embodiments, the modular readrRNA molecule and/or any components thereof are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having praline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin 3 signal peptide sequence, polyarginine peptide Args sequence, Guanine rich-molecular transporters, and sweet arrow peptide.

Yet another means of introducing one or more components of the modular readrRNA molecule systems as provided herein to the cell is by using SEND (see, e.g., Segel, M. et al., 2021. Science 373:6557; 882-889, the contents of which are hereby incorporated by reference in its entirety). In such embodiments, retroviral-like proteins, such as PEG10, which directly binds to and secretes its own mRNA in extracellular virus-like capsids, are pseudotyped with fusogens to deliver functional mRNA cargos (i.e., a modular readrRNA molecule as provided herein) to mammalian cells.

Other embodiments of the present disclosure provide for modification of a modular readrRNA molecule as provided herein. Such modifications may be for any purpose, such as increased stability, ability of the modular readrRNA molecule to evade the subject's immunity, and the like. For example, in instances where the modular readrRNA molecule comprises a circular RNA molecule, one such modification may include N6-methyladenosine modification. For example, inclusion of an N6-methyadenosine reader YTHDF2 sequence enables the sequestration of N6-methyladenosine-circularRNA thereby allowing for the suppression of innate immunity (see, e.g., Chen, Y. G. et al., (2019) Molecular Cell (76): 1; 96-109, the contents of which are hereby incorporated by reference in its entirety). In other embodiments, such modifications may include the replacing of uridine with pseudouridine to help evade the immune system of a subject (see. e.g., Dolgin, E. (2021) Nature 597; 318-324, the contents of which are hereby incorporated by reference in its entirety).

### P. Pharmaceutical Compositions

In another aspect, the present disclosure provides compositions comprising one or more of the modular readrRNA molecules as described herein, or a delivery system comprising a modular readrRNA molecule as provided herein (herein used singly or together as “molecules”) and an appropriate carrier, excipient or diluent. The exact nature of the carrier, excipient or diluent will depend upon the desired use for the compositions and may range from being suitable or acceptable for veterinary uses to being suitable or acceptable for human use. The compositions may optionally include one or more additional compounds and/or therapeutic agents.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

Alternatively, other pharmaceutical delivery systems may be employed. Liposomes and emulsions are well-known examples of delivery vehicles that may be used to deliver molecule(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the molecule(s). The pack may, for example, comprise metal or plastic foil, such as a blister pack. The pack or dispenser device may be accompanied by instructions for administration. The molecule(s) described herein, or pharmaceutical compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

Determination of an effective dosage of molecule(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound (e.g., efRNA product) that is at or above an IC50 of the particular compound as measured in as in vitro assay.

Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will depend upon, among other factors, the activity of the active compound, the bioavailability of the compound, its metabolism kinetics and other pharmacokinetic properties, the mode of administration, and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### Q. Cell Manipulation

In order to monitor and/or change the physiology of a cell, or group of cells of interest, the cell(s) of interest are identified on the basis of differential expression of one or more RNA transcripts the cell(s)'s through the SES component of the readrRNA. Through ADAR mediated editing, the operably linked effector molecule(s) are translated and may label the cell fluorescently, and/or effect some desired change in the physiology of the cell(s), including cell death.

In addition to the physical linkage of the SES component of the readrRNA to the encoded effector of the readrRNA, the cells of interest can further comprise a second nucleic acid entity that is under the control of the encoded effector of the readrRNA, comprising a system called a CellREADR system. For example the effector of the ReadrRNA can encode a transactivator that can activate genes either encoded on a second nucleic entity, where the second nucleic entity is endogenous to the cell encoding a gene under the control of the transactivator that is endogenous to the cell, and/or where the second nucleic entity is exogenously added to the cell and encodes a gene(s) under the control of the transactivator that is exogenous and/or endogenous to the cell.

A transactivator or repressor can also silence or decrease expression of specified endogenous genes in a cell by controlling the expression of exogenous genes encoding tight hairpin loops (shRNA) that silence. It is also noted that an alternative to being located on a second nucleic entity, genes under control of the transactivator optionally can be positioned on the readrRNA molecule itself. In the case of cells comprising a mutated gene that is ultimately causing a disease or disorder in a patient, the effector can encode a functioning gene and/or cause expression of nucleic encoding a functioning gene.

“hSyn” refers to a human synapsin 1 gene promoter, which is recognized in the art to confer highly neuron-specific long-term transgene expression from an adenoviral vector in the adult rat brain; “TRE-3G” refers to a eukaryotic inducible promoter; TRE is made up of Tet operator (tetO) sequence concatemers fused to a minimal promoter, (commonly the minimal promoter sequence derived from the human cytomegalovirus (hCMV) immediate-early promoter); In the absence of Tc or Dox, tTA binds to the TRE and activates transcription of the target gene; “mNeonGreen” is a basic (constitutively fluorescent) green/yellow fluorescent protein published in 2013, derived from Branchiostoma lanceolatum; “WPRE” is a woodchuck hepatitis virus post-transcriptional regulatory element (WPRE) increases transgene expression from a variety of viral vectors, although the precise mechanism is not known. WPRE is most effective when placed downstream of the transgene, proximal to the polyadenylation signal; “dTomato gene is a gene encoding dTomato, which is a basic (constitutively fluorescent) orange fluorescent protein derived from Discosoma sp;” tdTomato“is a genetic fusion of two copies of the dTomato gene; tdTomato is an exceptionally bright red fluorescent protein; “mCherry” is a basic (constitutively fluorescent) red fluorescent protein published in 2004, derived from Discosoma sp. It is reported to be a very rapidly-maturing monomer with low acid sensitivity; virally expressed mCherry is pseudocolored magenta. “SmV5” refers to spaghetti monster V5; “smFLAG” refers to Spaghetti Monster FLAG: 10 copies of an epitope tag FLAG-(DYKDDDDK); “tTA2” is a tetracycline dependent transcription activator; “W3SL” is truncated woodchuck hepatitis posttranscriptional regulatory element and polyadenylation signal cassette (Choi et al., 2014). Choi J H, Yu N K, Baek G C, Bakes J, Seo D, Nam H J, Baek S H, Lim C S, Lee Y S, Kaang B K (2014); eYFP is enhanced yellow fluorescent protein. AMPA receptors (AMPARs) are excitatory neurotransmitter receptors and composed of four subunits (GluA1-4), with each subunit being coded by a different gene; “CAG” promoter which is a hybrid construct consisting of the cytomegalovirus (CMV) early enhancer fused to the chicken beta-actin promoter, and is a strong promoter for recombinant expression in HEK293F cells. Unless otherwise defined, all technical terms used herein have the same meaning as commonly understood by one of ordinary skill in the art to which this disclosure belongs.

Accordingly, cells or tissues express the modular readrRNA molecules, and systems comprising such modular readrRNA molecules. Hence, another aspect of the present disclosure provides a cell comprising a modular readrRNA molecule as provided, or a delivery system comprising a modular readrRNA molecule as provided herein. The readrRNA molecules provided herein can be expressed in prokaryotic and eukaryotic cells. In some embodiments, the cell comprises a eukaryotic cell. In one embodiment, the eukaryotic cell comprises a mammalian cell or a plant cell.

Another aspect of the present disclosure provides an animal model or a plant model comprising the cell as provided herein.

### R. Treatment

The present disclosure further encompasses methods comprising a readrRNA molecule as provided herein and as provided in the Examples below.

One aspect of the present disclosure provides a method of treating a condition and/or disease in a subject in need thereof, the method comprising, consisting of, or consisting essentially of administering to the subject a modular readrRNA molecule as provided herein, or a delivery system comprising a modular readrRNA molecule as provided herein, such that the condition and/or disease is treated in the subject.

In some embodiments, the condition and/or disease is selected from the group consisting of cachexia, including cancer cachexia, obesity, cancer, infectious disease, a genetic disorder, and the like.

As used herein, “treating” of a condition and/or disease is ameliorating any condition or symptom associated with the condition and/or disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic. In some embodiments of any of the aspects, the administration is subcutaneous.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The effects of any particular dosage can be monitored by a suitable bioassay, e.g., assay for tumor growth and/or size among others. The dosage can be determined by a physician and adjusted, as necessary, to suit observed effects of the treatment.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample may be obtained directly from a subject (e.g., by blood or tissue sampling) or from a third party (e.g., received from an intermediary, such as a healthcare provider or lab technician).

As used herein the term “condition and/or disease” includes, but is not limited to, any abnormal condition and/or disorder of a structure or a function that affects a part of an organism. It may be caused by an external factor, such as an infectious disease, or by internal dysfunctions, such as cancer, cancer metastasis, genetic disorders/mutations (both congenital and environmental) and the like.

As used herein, a monogenic somatic cell disorder comprising an underlying genetic mutation in a gene, refers to a monogenetic disorder caused by a variant in a single gene. The variant may be present on one or both chromosomes of a pair. Nonlimiting examples of monogenic disorders are cystic fibrosis, Huntington's disease and sickle cell disease.

As is known in the art, a cancer is generally considered as uncontrolled cell growth. The methods of the present invention can be used to treat any cancer, and any metastases thereof, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma.

“Contacting” as used herein, e.g., as in “contacting a sample” refers to contacting a sample or cell directly or indirectly in vitro, ex vivo, or in vivo (i.e. within a subject as defined herein). Contacting a sample may include addition of a compound (e.g., a readrRNA molecule as provided herein and/or a delivery system comprising a readrRNA molecule as provided herein) to a sample, or administration to a subject. Contacting encompasses administration to a solution, cell, tissue, mammal, subject, patient, or human. Further, contacting a cell includes adding an agent to a cell culture.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer. A subject can be male or female.

A subject can be one who has been previously diagnosed with or identified as suffering from or having a condition in need of treatment (e.g. cancer) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition, or one or more complications related to the condition or a subject who does not exhibit risk factors.

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom or contribute to faster recovery from the disease or symptom. Accordingly, the methods described herein can be prophylactic relative to metastasis or tumor formation. In some embodiments of any of the aspects, prophylactic treatment is not prevention of all symptoms or signs of a disease.

A “subject in need” of treatment for a particular condition can be a subject having that condition, diagnosed as having that condition, or at risk of developing that condition.

The terms “decrease” “reduced”, “reduction”, and “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder. The term “statistically significant” or “significantly” refers to statistical significance and generally means a two standard deviation (2SD) or greater difference.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level. The term “statistically significant” or “significantly” refers to statistical significance and generally means a two standard deviation (2SD) or greater difference.

Another aspect of the present disclosure provides a method of detecting the presence or dynamics of cell state-defining cellular RNA and/or switching on the translation of one or more effector proteins, the method comprising, consisting of, or consisting essentially of detecting/hybridizing the target effector RNA with a modular readrRNA molecule as provided herein, or a delivery system comprising a modular readrRNA molecule as provided herein, or a pharmaceutical composition as provided herein, in which the sensor domain detects and binds a specific cell type RNA through sequence-specific base pairing, the one or more ADAR-editable STOP codons act as a translation switch thereby allowing for the translation of the effector RNA that encodes for the effector protein.

In some embodiments, the effector proteins are within a cell.

Detecting/assessing a dynamic state of a cell is critical to detecting a disease in an individual, for diagnosis. Detecting/assessing a dynamic state of a cell is critical to a targeted treatment of a disease in an individual by providing a therapeutic specifically to specified targeted cells where the therapeutic can be most effective and with reduced pleiotropic side effects. However, the effector molecules can subsequently function outside the specified cell, for example in the case of secreted cytokines and interleukins (e.g., IL-6), and T-CARs.

Another aspect of the present disclosure provides a method for treating a disease or disorder in a mammal, the method comprising, consisting of, or consisting essentially of: providing an agent, said agent comprising a modular RNA molecule as provided herein, or a nucleic acid composition as provided herein, or a delivery vehicle as provided herein; and administering said agent to said mammal in a therapeutically effective amount to permit translation of said 3′ encoded protein or said effector protein in selected cells of said mammal, thereby to produce said protein in said cells, wherein production of said protein in said cells provides for treatment of said disease or disorder in said mammal.

In one embodiment, the agent comprises said composition of as provided herein, or said delivery vehicle as provided herein is administered and said first protein coding region encoding said effector protein comprised in said agent encodes a transactivator protein that activates expression of said second protein coding region, and wherein expression of said second protein coding region in said selected cells increases the activity of Gfa1-expressing AP neurons and is thereby therapeutically effective in treating said disease or disorder.

In some embodiments, the disease or disorder comprises obesity.

In other embodiments, the agent comprises said composition of as provided herein, or said delivery vehicle as provided herein is administered and said first protein coding region encoding said effector protein comprised in said agent encodes a transactivator protein that activates expression of said second protein coding region, and wherein expression of said second protein coding region in said selected cells decreases the activity of Gfa1-expressing AP neurons and is thereby therapeutically effective in treating said disease or disorder.

In some embodiments, the disease or disorder comprises cachexia. In certain embodiments, the cachexia comprises cancer cachexia.

### S. Kits

The present disclosure further provides kits comprising the compositions provided herein and for carrying out the subject methods as provided herein. For example, in one embodiment, a subject kit may comprise, consist of, or consist essentially of one or more of the following: (i) a modular readrRNA molecule as provided herein; (ii) a CellREADR system as provided herein; (iii) delivery systems comprising a modular readrRNA and/or CellREADR system as provided herein; (iv) cells comprising a modular readrRNA and/or CellREADR system and/or delivery system comprising a modular readrRNA and/or CellREADR system as provided herein; and/or (v) pharmaceutical compositions as provided herein.

In other embodiments, a kit may further include other components. Such components may be provided individually or in combinations and may provide in any suitable container such as a vial, a bottle, or a tube. Examples of such components include, but are not limited to, (i) one or more additional reagents, such as one or more dilution buffers; one or more reconstitution solutions; one or more wash buffers; one or more storage buffers, one or more control reagents and the like, (ii) one or more control expression vectors or RNA polynucleotides; (iii) one or more reagents for in vitro production and/or maintenance of the of the molecules, cells, delivery systems etc. provided herein; and the like. Components (e.g., reagents) may also be provided in a form that is usable in a particular assay, or in a form that requires addition of one or more other components before use (e.g., in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof. In some embodiments, the kits disclosed herein comprise one or more reagents for use in the embodiments disclosed herein.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

That is, a kit is an assemblage of materials or components, including at least one reagent described herein. The exact nature of the components configured in the kit depends on its intended purpose. In some embodiments of any of the aspects, a kit includes instructions for use. “Instructions for use” typically include a tangible expression describing the technique to be employed in using the components of the kit, e.g., to treat a subject or for administration to a subject. Still in accordance with the present invention, “instructions for use” may include a tangible expression describing the preparation of at least one reagent described herein, such as dilution, mixing, or incubation instructions, and the like, typically for an intended purpose.

Optionally, the kit also contains other useful components, such as, measuring tools, diluents, buffers, syringes, pharmaceutically acceptable carriers, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging may also preferably provide an environment that protects from light, humidity, and oxygen. As used herein, the term “package” refers to a suitable solid matrix or material such as glass, plastic, paper, foil, polyester (such as polyethylene terephthalate, or Mylar) and the like, capable of holding the individual kit components. Thus, for example, a package can be a glass vial used to contain suitable quantities of a composition containing a volume of at least one reagent described herein. The packaging material generally has an external label which indicates the contents and/or purpose of the kit and/or its components.

Another aspect of the present disclosure provides all that is described and illustrated herein.

### DETAILED DESCRIPTION

Cancer-associated cachexia is a devastating metabolic wasting syndrome characterized by anorexia, fatigue, and dramatic involuntary bodyweight loss^{18,19,24,25}. It affects 50-80% of cancer patients, lowering the quality of life, reducing tolerance to anticancer therapies, and drastically accelerating death^{19,20}. The brain is known to have an important role in the pathogenesis of cancer-associated cachexia¹⁶⁻¹⁸. In particular, recent studies implicate the hypothalamus, parabrachial nucleus, area postrema and other hindbrain structures in the development of cachectic phenotypes in animal models of cancer, such as anorexia, weight loss, and accelerated catabolic processes²⁶⁻³²

However, it is not well understood how the brain senses and reacts to peripheral cancers, and its subsequent contribution to the development of cachectic phenotypes.

Possible mediators of cancer-associated cachexia that may act as messengers to engage the brain during cancer progression include tumor-derived factors, metabolites from organs indirectly affected by tumor, and immune or inflammatory factors altered by tumor^{16-18,24,33,34} One such messenger is the pleiotropic cytokine IL-6^{18-20,23,24,35,36} Indeed, elevated levels of circulating IL-6 are associated with cancer progression and cachexia in patients and animal models^{1-5,15}. Systemic administration of antibodies against IL-6 or IL-6 receptor shows anticachectic effects in human case reports^{6,7,37,38}. Consistently, cancer-associated cachexia in mouse models can be ameliorated by peripheral administration of antibodies against IL-6⁸⁻¹³ or IL-6 receptor¹⁴, or by deletion of the IL6 gene^{9,10.}

These findings strongly indicate that IL-6 is a key mediator of cancer-associated cachexia.

Most studies and therapeutic explorations on IL-6 in cancer-associated cachexia has focused on its functions in peripheral organs, including the skeletal muscle, liver, and gut²³. Previous studies suggest that IL-6 may also influence brain functions-such as the regulation of food intake³⁹⁻⁴¹, fever⁴² and the hypothalamic-pituitary-adrenal (HPA) axis⁴³. However, it is unclear how peripheral IL-6 is involved in these functions.

Thus, Interleukin-6 (IL-6) has been long considered a key player in cancer-associated cachexia¹⁻¹⁵, and it is believed that sustained elevation of IL-6 production during cancer progression causes brain dysfunctions, which ultimately result in cachexia¹⁶⁻²⁰. However, how peripheral IL-6 influences the brain remains poorly understood.

Conceivably, IL-6 could activate its receptors on the terminals of peripheral nerves, which then transmit the signals to the brain⁴⁴. Alternatively, circulating IL-6 may cross the blood-brain barrier (BBB) or reach circumventricular organs that lack or have a weak BBB, thereby acting within the brain^{43,45,46}.

In the working examples herein, we show that neurons in the area postrema (AP), a circumventricular structure in the hindbrain, mediate the function of IL-6 in cancer-associated cachexia in mice.

The following Examples are provided by way of illustration and not by way of limitation.

### Working Example I

Part A. Gfral (GDNF Family Receptor Alpha like) CellREADR Strategy.

The genomic locus of the mouse Gfral is 49,556 base pairs, with a coding sequence of 1182 base pairs. See FIG. 29. GDF-15 is a ligand of Gfral .

CellREADR is a single modular readrRNA molecule, consisting of a translationally in-frame 5′-sensor domain (sesRNA) and 3′-effector domain (efRNA), separated by a T2a coding region. sesRNA is complementary to a cellular RNA target and contains an in-frame STOP codon that prevents efRNA translation. Base pairing between the sesRNA component of CellREADR and the target RNA recruits ADARs, which mediate A->I editing and convert the UAG STOP to a UGG Trp codon, switching on translation of effector protein.

The Gfral coding RNA sequence was used to design 11 sesRNAs that target the mouse Gfral coding RNA. See FIG. 21.

The 11 sesRNAs were designed to target mouse Gfral coding sequence. Each line across indicates one SESRNA targeting region to Gfral coding sequences. Some sesRNAs contain spreading fragments for multiple targeting, with one or two stops in the middle fragment.

Thus, the RNA sensor comprises at least one avidity binding region. In some embodiments, the RNA sensor comprises at least two or three avidity binding regions. In some embodiments, the RNA sensor comprises at least four or five avidity binding regions. In some embodiments, the RNA sensor comprises at least six or seven avidity binding regions. In some embodiments, the RNA sensor comprises more than seven avidity binding regions. In some embodiments, the avidity binding regions are separated by a MS2 hairpin region.

TABLE 4
Gra1 sesRNA design
1182 bp

| ID# | Length | Stop Codon No. | Tentacle Frag# |
|---|---|---|---|
| ses1 | 285 | 1 | No |
| ses2 | 315 | 1 | No |
| ses3 | 315 | 1 | No |
| ses4 | 315 | 2 | No |
| ses6 | 291 | 2 | No |
| ses7 | 447 | 1 | 5 |
| ses8 | 414 | 1 | 4 |
| ses9 | 495 | 1 | 4 |
| ses10 | 423 | 1 | 5 |
| ses11 | 564 | 2 | 6 |

| | |
|---|---|
| Ses1 | ggagaaccctggacctgaattcggcaagtggggggtggaactatattcagtgcac |
| | Atggcttgctgtgaagagtttctttggattgctgacagggtatatcagattgagc |
| | Acagtcacaaaaagccaacatctgggcagtgtgaaaaggcatattttgatagaag |
| | aaccgtatAgctgcttggcagtgtttcacatcacacagatttccatttgctgagc |
| | gtgcttgaaggtaaagggccaactgtgcattacaaaccacatcccctacacacgc |
| | ctctgtcacctccaaacaagactgcatctgttggcgcgccagagggcagaggaagt |
| | cttcta (SEQ ID NO: 12) |
| Ses2 | ggagaaccctggacctgaattcCAACTGTGCATTACAAACCACATCCCCTACACAC |
| | GCCTCTGTCACCTCCAAACAAGACTGCATCTGTTTGAATCCATGATAGAAAGGAGT |
| | AGTTTAGATTCCATTTATCTTTATTGTCCTTTTCCATGTTATCTGTCTTATTAGTG |
| | CACTTTTTTCCAAAAAGTTTGTTTACTGTACAGTGGAGGTCATCCATACAAAGACAC |
| | TCTTTTAAATTGGAAATTTTTTTCCACCAAAGCCTGGATACTCAGGTTACAATGTAG |
| | TGAATTATTTATCTTGCAGGAGTCACCTGGAGTAAGGCAGGTGTCTTCCATTGATggc |
| | gcgccagagggcagaggaagtcttcta (SEQ ID NO: 13) |
| Ses3 | ggagaaccctggacctgaattcCATGCTAATGCAGGTCTCATCTTCATGGCACTTCCC |
| | AGTTATGTGGGGCCAGCATTCTGTCTGGAATGTTCGGTAGTGTGTCCTGCAGAATTCA |
| | TCATTTCGGCAAGTGTGAATTACACTGAGGCAAGTGGGGGGTGGAACTATATTCAGTG |
| | CACATAGCTTGCTGTGAAGAGTTTCTTTGGATTGCTGACAGGGTATATCAGATTGAGC |
| | ACAGTCACAAAAAGCCAACATCTGGGCAGTGTTAAAAGGCATATTTTGATAGAAGAACC |
| | GTATGGCTGCTTGGCAGTGTTTCACATCACACAGATTTCCATTTGCggcgcgccagaggg |
| | Cagaggaagtcttcta (SEQ ID NO: 14) |
| Ses4 | ggagaaccctggacctgaattcCATGCTAATGCAGGTCTCATCTTCATGGCACTTCCCA |
| | GTTATGTGGGGCCAGCATTCTGTCTGGAATGTTCGGTAGTGTGTCCTGCAGAATTCATCA |
| | TTTCGGCAAGTGTGAATTACACTGAGGCAAGTGGGGGGTAGAACTATATTCAGTGCACAT |
| | AGCTTGCTGTGAAGAGTTTCTTTGGATTGCTGACAGGGTATATCAGATTGAGCACAGTCA |
| | CAAAAAGCCAACATCTGGGCAGTGTTAAAAGGCATATTTTGATAGAAGAACCGTATGGCT |
| | GCTTGGCAGTGTTTCACATCACACAGATTTCCATTTGCggcgcgccagagggcagaggaa |
| | Gtcttcta (SEQ ID NO: 15) |
| Ses5 | ggagaaccctggacctgaattcAAAGGAGTAGTAGTTAGATTCCATTTATCTTTATTGTC |
| | CTTTTCCATGTTATCTGTCTTATTGGTGCACTTTTTTCCAAAAAGTTTGTTTACTGTACA |
| | GTGGAGGTCATCCATACAAAGACACTCTTTAAATTGGAAATTTTTTTCCACCAAAGCCTA |
| | GATACTCAGGTTACAATGTAGTGAATTATTTATCTTGCAGGAGTCACCTGGAGTAAGGCA |
| | GGTGTCTTCCATTGATCTCCATGACTGCTCACAGCCATTTGCATCAATCAAGCATTTCTG |
| | TATTAAATGTGCACAATCATTTGTTTGGGAAGAGGATTCATTTTCTGAGCTTAACGTAAC |
| | AGCCAGGAAAAggcgcgccagagggcagaggaagtcttcta (SEQ ID NO: 16) |
| Ses6 | ggagaaccctggacctgaattcAAAGGAGTAGTAGTTAGATTCCATTTATCTTTATTGTC |
| | CTTTTCCATGTTATCTGTCTTATTGGTGCACTTTTTTCCAAAAAGTTTGTTTACTGTACA |
| | GTGGAGGTCATCCATACAAAGACACTCTTTAAATTAGAAATTTTTTTCCACCAAAGCCTA |
| | GATACTCAGGTTACAATGTAGTGAATTATTTATCTTGCAGGAGTCACCTGGAGTAAGGCA |
| | GGTGTCTTCCATTGATCTCCATGACTGCTCACAGCCATTTGCATCAATCAAGCATTTCT |
| | GTATTAAATGTGCAggcgcgccagagggcagaggaagtcttcta (SEQ ID NO: 17) |
| Ses7 | ggagaaccctggacctgaattcGATGAGGGATCTCTTTTTTCACTTTGTATCCTTAACT |
| | TGAGCATCACCAAGAAAAGAATTCCACAGaaaTGCTGGAAAATCATGCACACGTGTTCT |
| | TCAGCCTGTGTAACGCCCCTGCAAGCACAGGGTaaaCAGGTCTCATCTTCATGGCACTT |
| | CCCAGTTATGTGGGGCCAGCATTCTGTCTGGAATGTTCGGaaaagtcacaaaaagccaa |
| | catctgggcagtgtgaaaaggcatattttgatagaagaaccgtatAgctgcttggcagt |
| | gtttcacatcacacagatttccatttgctgagcgtgcttgaaggtaaaTTATCTGTCTT |
| | ATTGGTGCACTTTTTTCCAAAAAGTTTGTTTACTGTACAGTGGAGGTCAaaaTGTGCAC |
| | AATCATTTGTTTGGGAAGAGGATTCATTTTCTGAGCTTAACGTAACAGCCAGGAAAggc |
| | gcgccagagggcagaggaagtcttcta (SEQ ID NO: 18) |
| Ses8 | ggagaaccctggacctgaattcGATGAGGGATCTCTTTTTTCACTTTGTATCCTTAACT |
| | TGAGCATCACCAAGAAAAGAATTCCACAGaaaTGCTGGAAAATCATGCACACGTGTTCT |
| | TCAGCCTGTGTAACGCCCCTGCAAGCACAGGGTaaaAGCATTCTGTCTGGAATGTTCGG |
| | TAGTGTGTCCTGCATAATTCATCATTTCGGCAAGTGTaaaTCTGTCTTATTGGTGCACT |
| | TTTTTCCAAAAAGTTTGTTTACTGTACAGTGGAGGTCATCCATACAAAGACACTCTTTA |
| | AATTAGAAATTTTTTTCCACCAAAGCCTGGATACTCAGGTTACAATGTAGTGAATTATT |
| | TATCTTGCAGGAGTCAaaaTGTGCACAATCATTTGTTTGGGAAGAGGATTCATTTTCTGA |
| | GCTTAACGTAACAGCCAGGAAAggcgcgccagagggcagaggaagtcttcta (SEQ ID |
| | NO: 19) |
| Ses9 | ggagaaccctggacctgaattcGATGAGGGATCTCTTTTTTCACTTTGTATCCTTAACTT |
| | GAGCATCACCAAGAAAAGAATTCCACAGaaaTGCTGGAAAATCATGCACACGTGTTCTTC |
| | AGCCTGTGTAACGCCCCTGCAAGCACAGGGTaaaATAATTCATCATTTCGGCAAGTGTGA |
| | ATTACACGGAGGCAAGTGGGGGGTGGAACTATATTCAGTGCACATGGCTTGCTGTGAAGA |
| | GTTTCTTTAGATTGCTGACAGGGTATATCAGATTGAGCACAGTCACAAAAAGCCAACATC |
| | TGGGCAGTGTGAAAAGGCATATTTTGATAGAAGAACCGTATGGCTGCTTGGCAGTGTTTC |
| | ACATCACACAGATTTCCATTTGCTGAGCaaaTTATCTGTCTTATTGGTGCACTTTTTTCC |
| | AAAAAGTTTGTTTACTGTACAGTGGAGGTCAaaaTGTGCACAATCATTTGTTTGGGAAGA |
| | GGATTCATTTTCTGAGCTTAACGTAACAGCCAGGAAAggcgcgccagagggcagaggaag |
| | tcttcta (SEQ ID NO: 20) |
| Ses10 | ggagaaccctggacctgaattcGATGAGGGATCTCTTTTTTCACTTTGTATCCTTAACTT |
| | GAGCATCACCAAGAAAAGAATTCCACAGaaaTGCTGGAAAATCATGCACACGTGTTCTT |
| | CAGCCTGTGTAACGCCCCTGCAAGCACAGGGTaaaGGGGTGGAACTATATTCAGTGCAC |
| | ATGGCTTGCTGTGAAGAGTTTCTTTAGATTGCTGACAGGGTATATCAGATTGAGCACAG |
| | TCACAAAAAGCCAACAaaaGGTAAAGGGCCAACTGTGCATTACAAACCACATCCCCTAC |
| | ACACGCCTCTGTCACCTCCAaaaTTATCTGTCTTATTGGTGCACTTTTTTCCAAAAAGT |
| | TTGTTTACTGTACAGTGGAGGTCAaaaTGTGCACAATCATTTGTTTGGGAAGAGGATT |
| | CATTTTCTGAGCTTAACGTAACAGCCAGGAAAggcgcgccagagggcagaggaagtct |
| | tcta (SEQ ID NO: 21) |
| SES11 | ggagaaccctggacctgaattcGATGAGGGATCTCTTTTTTCACTTTGTATCCTTAA |
| | CTTGAGCATCACCAAGAAAAGAATTCCACAGaaaTGCTGGAAAATCATGCACACGTG |
| | TTCTTCAGCCTGTGTAACGCCCCTGCAAGCACAGGGTaaaCAGGTCTCATCTTCATGG |
| | CACTTCCCAGTTATGTGGGGCCAGCATTCTGTCTGGAATGTTCGGaaaCTGGAATGTT |
| | CGGTAGTGTGTCCTGCAGAATTCATCATTTCGGCAAGTGTGAATTACACTGAGGCAAG |
| | TGGGGGGTAGAACTATATTCAGTGCACATAGCTTGCTGTGAAGAGTTTCTTTGGATTG |
| | CTGACAGGGTATATCAGATTGAGCACAGTCACAAAAAGCCAACATCTGaaaGGTAAAGG |
| | GCCAACTGTGCATTACAAACCACATCCCCTACACACGCCTCTGTCACCTCCAaaaTTATC |
| | TGTCTTATTGGTGCACTTTTTTCCAAAAAGTTTGTTTACTGTACAGTGGAGGTCAaaaT |
| | GTGCACAATCATTTGTTTGGGAAGAGGATTCATTTTCTGAGCTTAACGTAACAGCCAGG |
| | AAAggcgcgccagagggcagaggaagtcttcta (SEQ ID NO: 22) |

The below schematic in FIG. 22 illustrates a procedure to screen the above sensors for Gfral mRNA in HEK cells.

READR^{Gfral-GFP} encodes a readrRNA consisting of a BFP (Blue Fluorescent Protein) sequence followed by sesRNA^{Gfral} and an effector protein, in this case green fluorescent protein (efRNAGFP).

FIG. 23 schematic provides experimental procedures for sesRNA^{Gfral} screening.

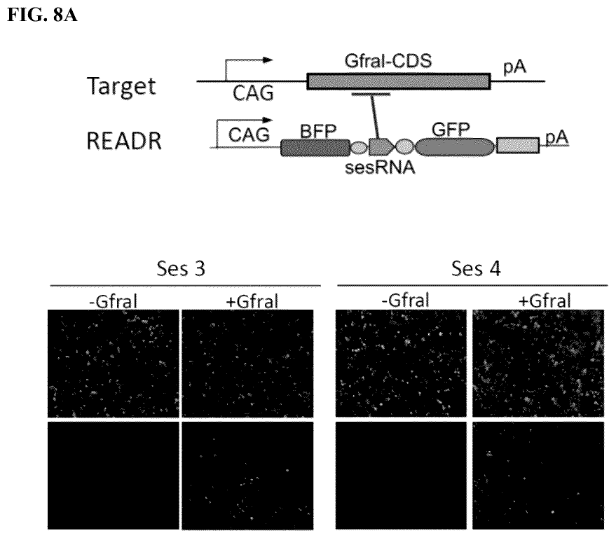

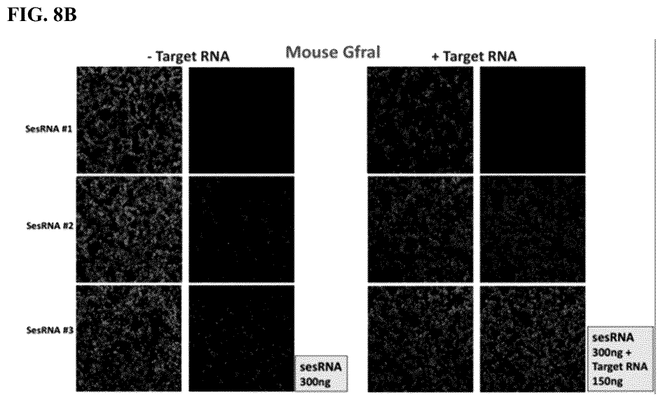

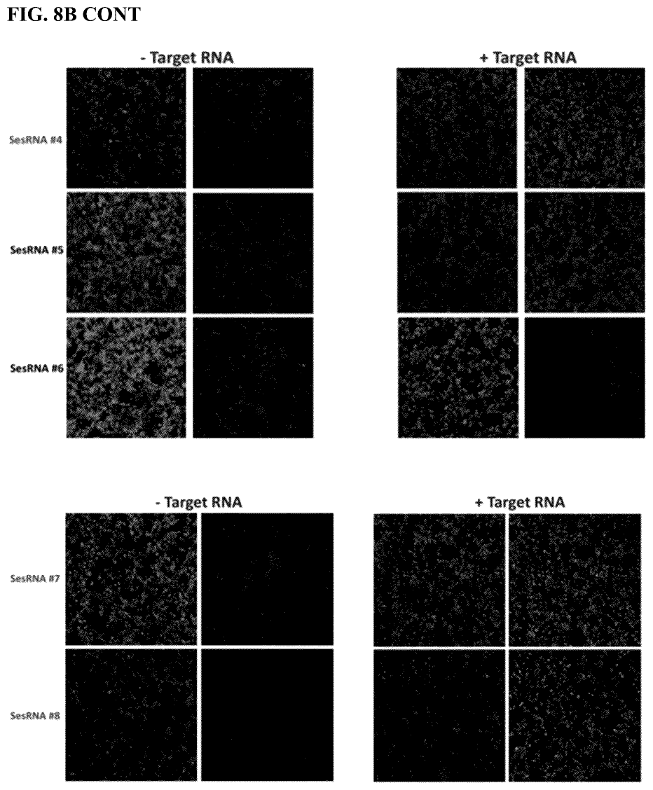

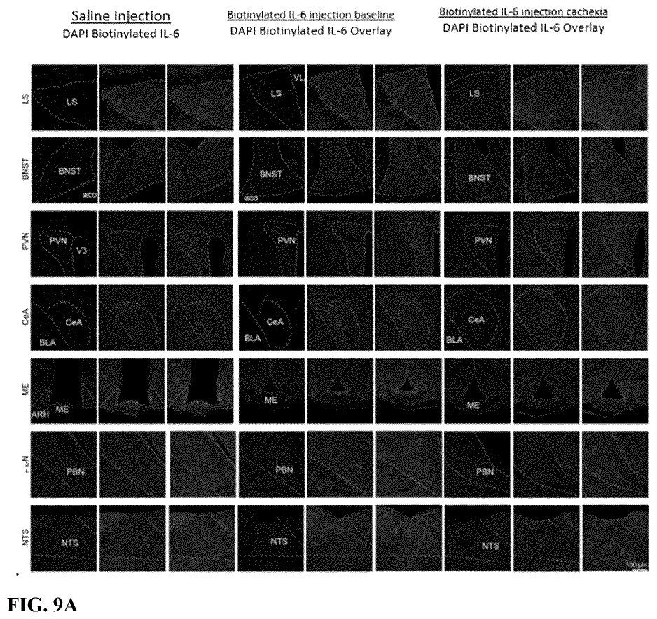

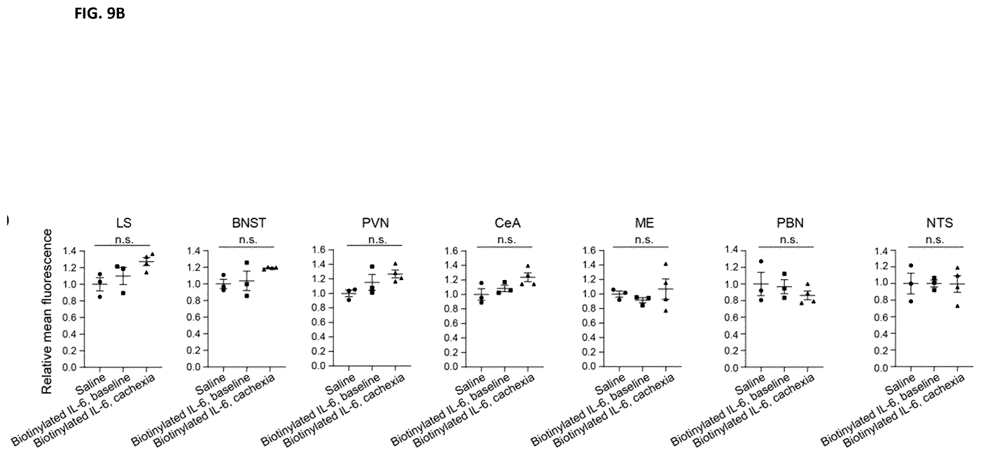

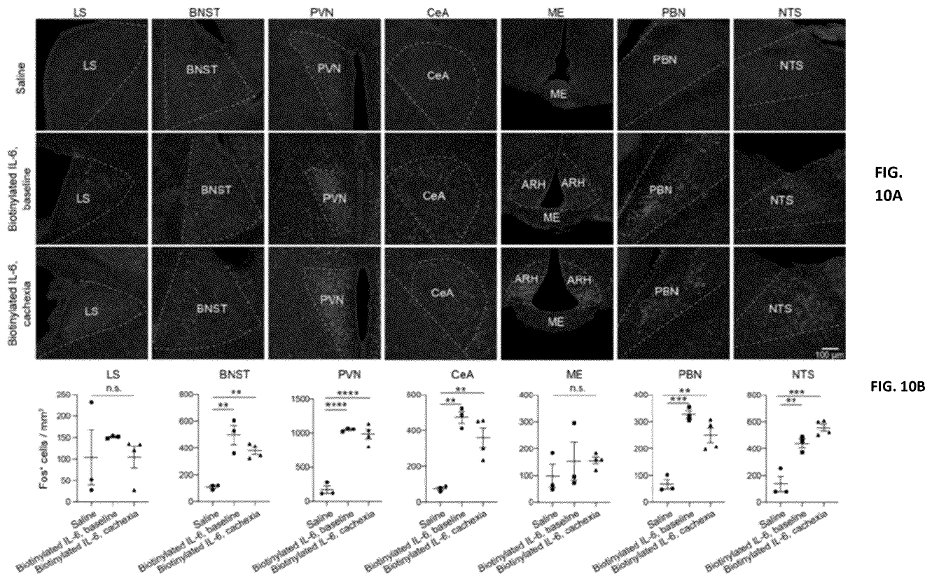

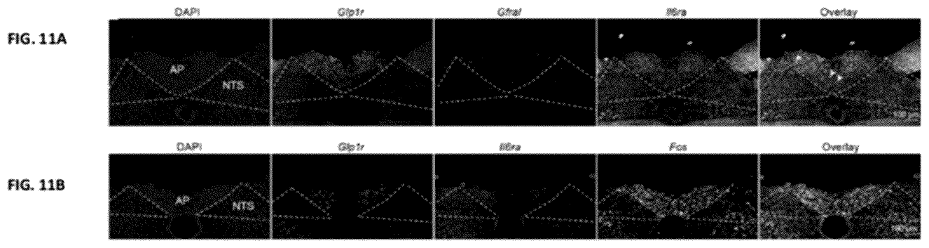

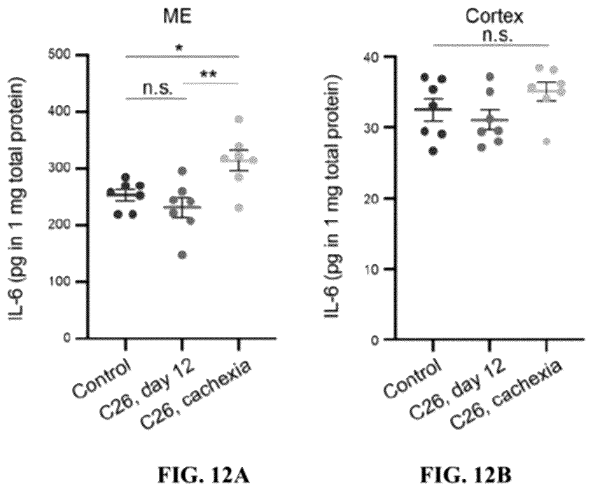

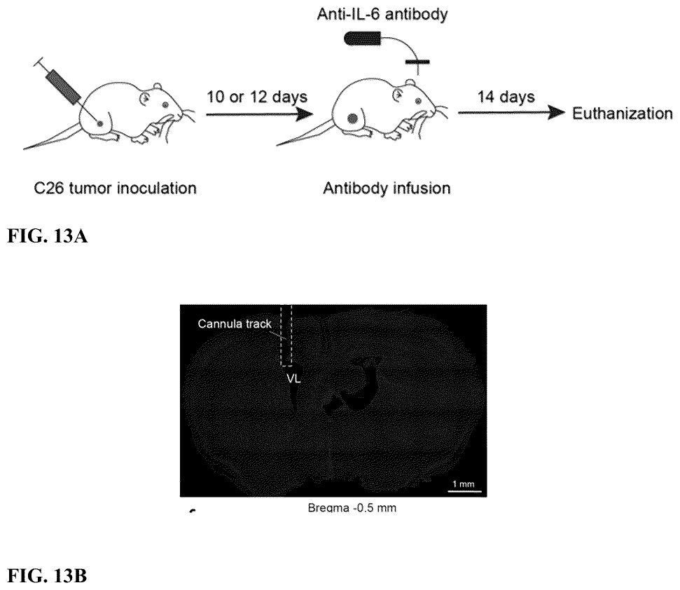

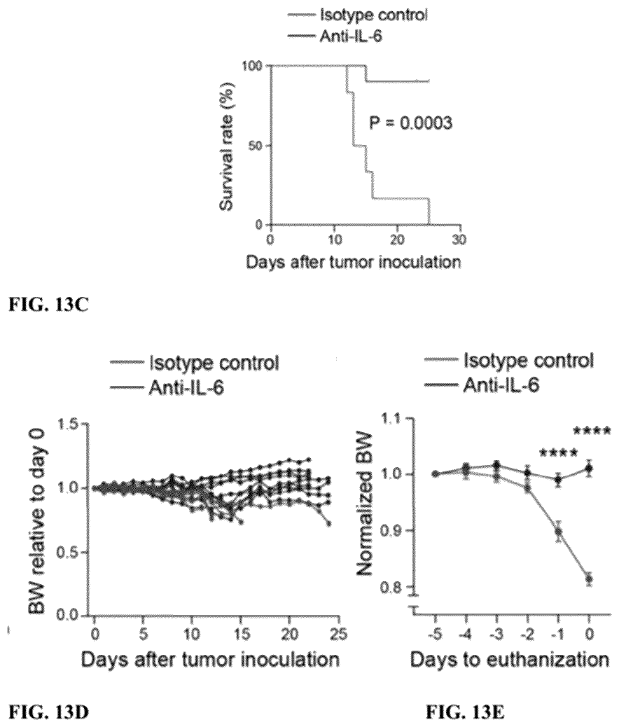

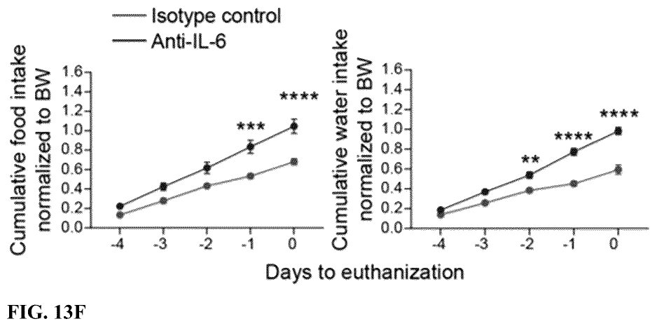

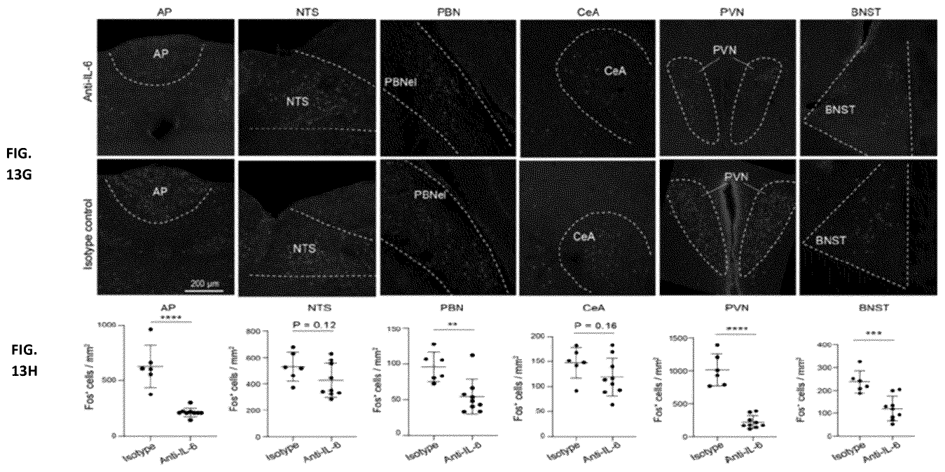

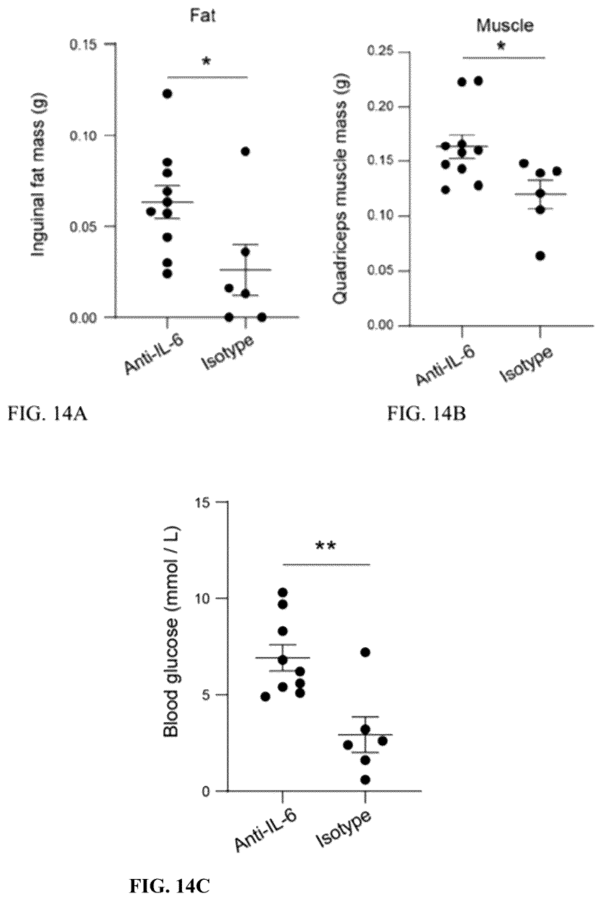

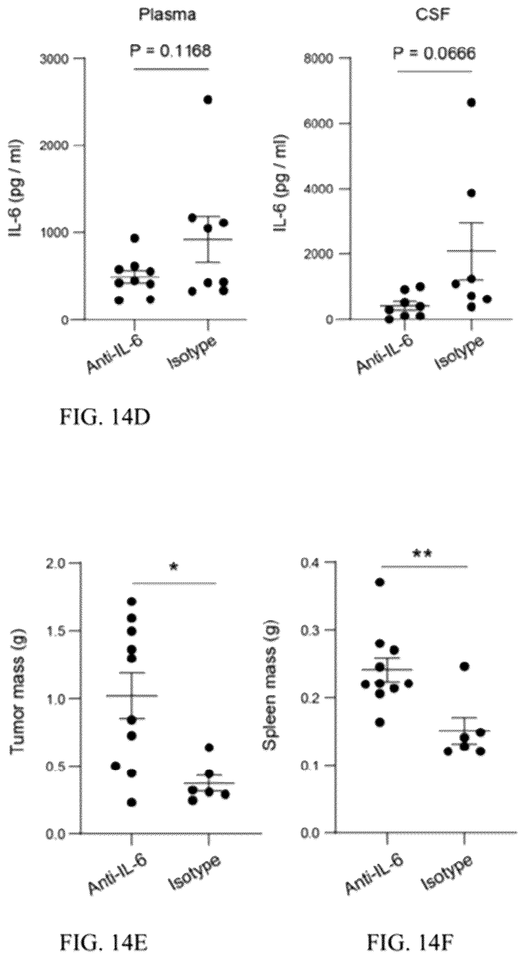

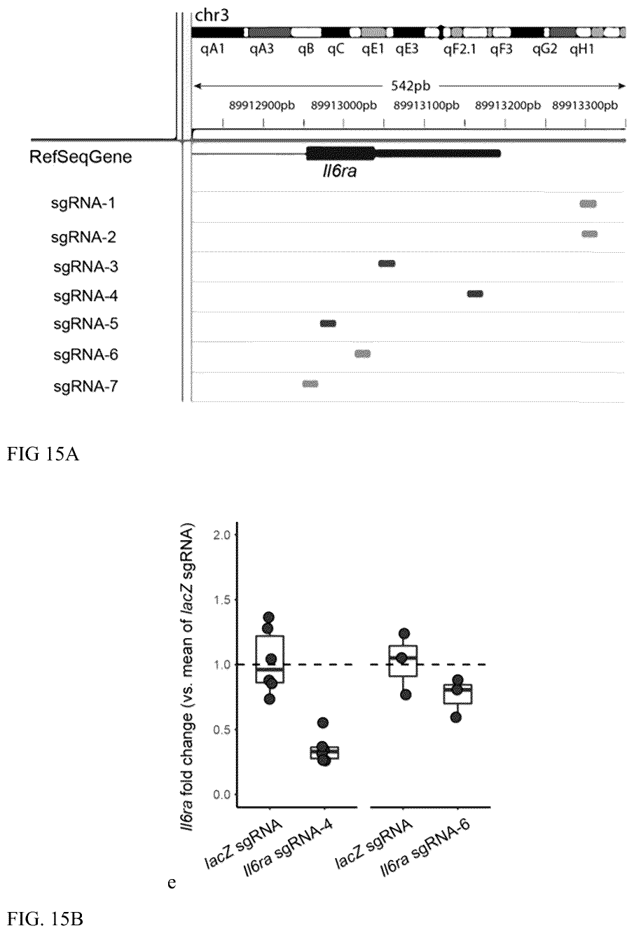

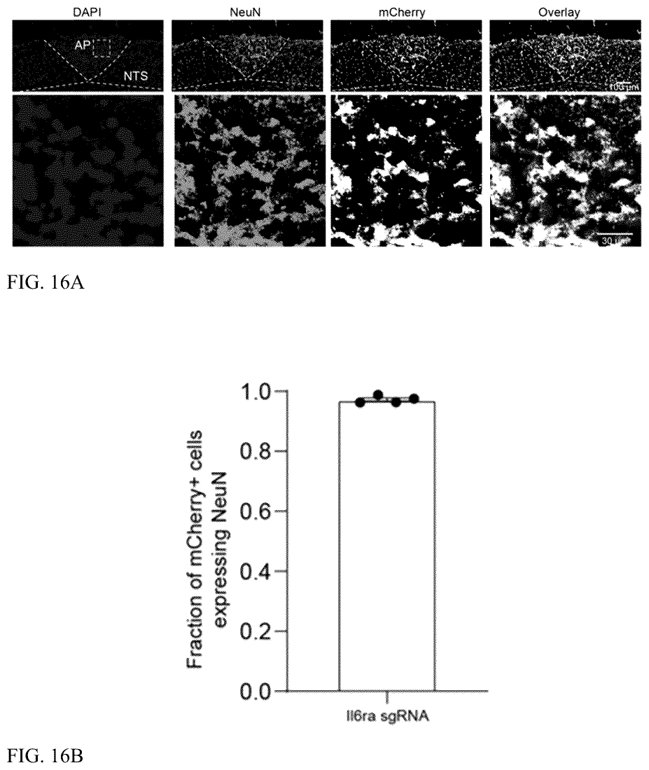

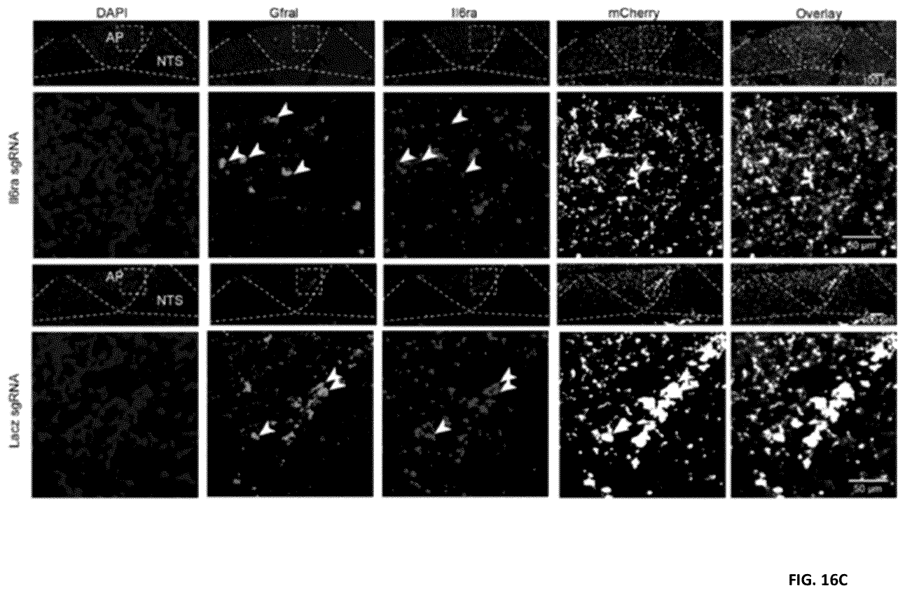

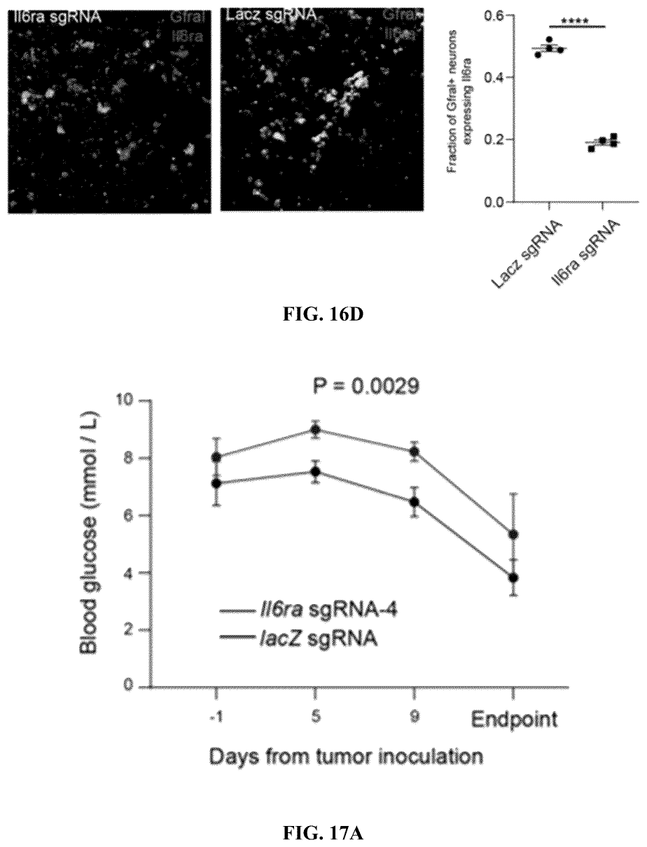

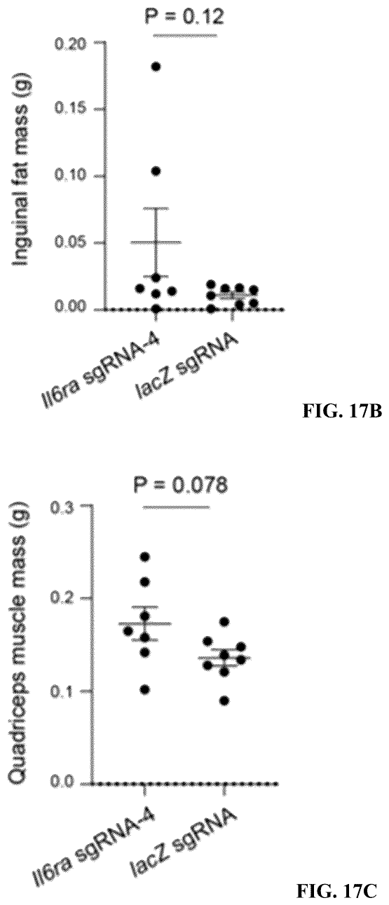

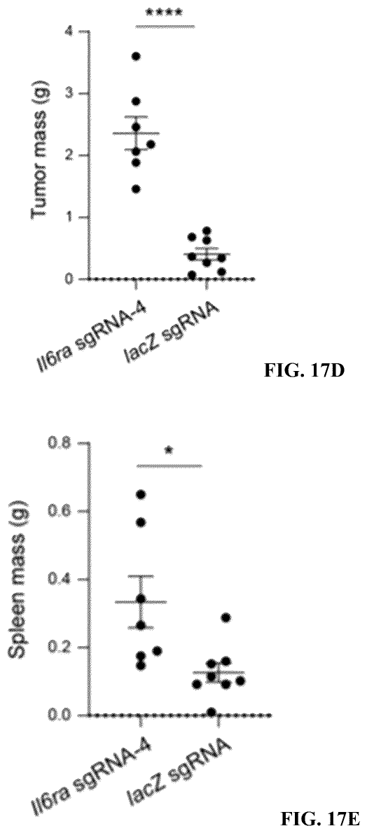

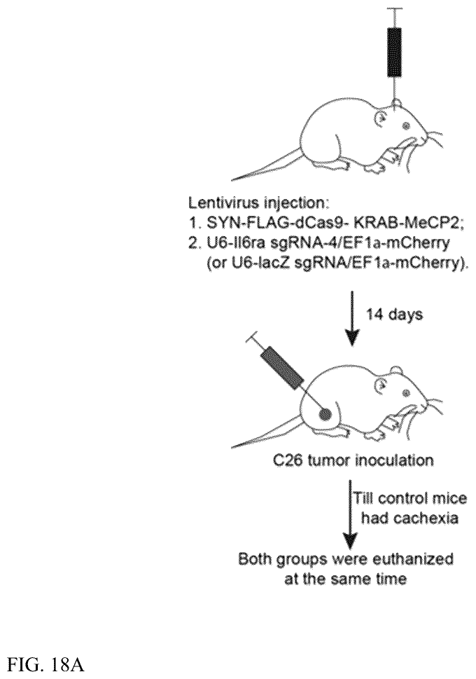

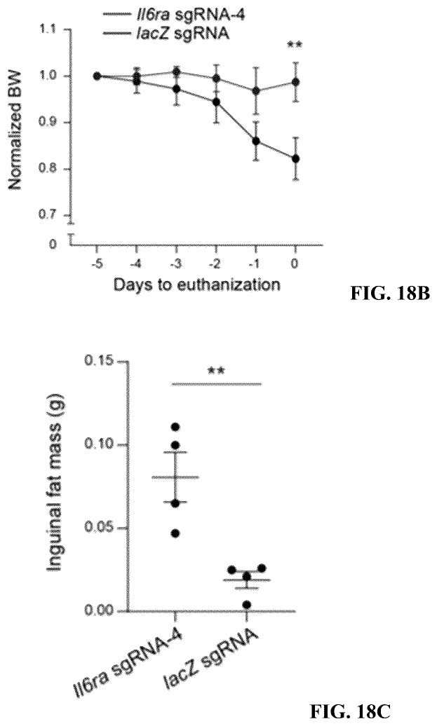

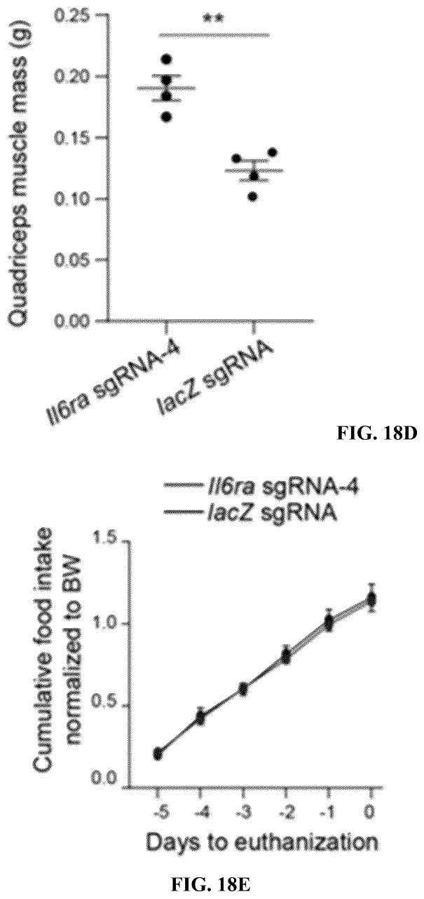

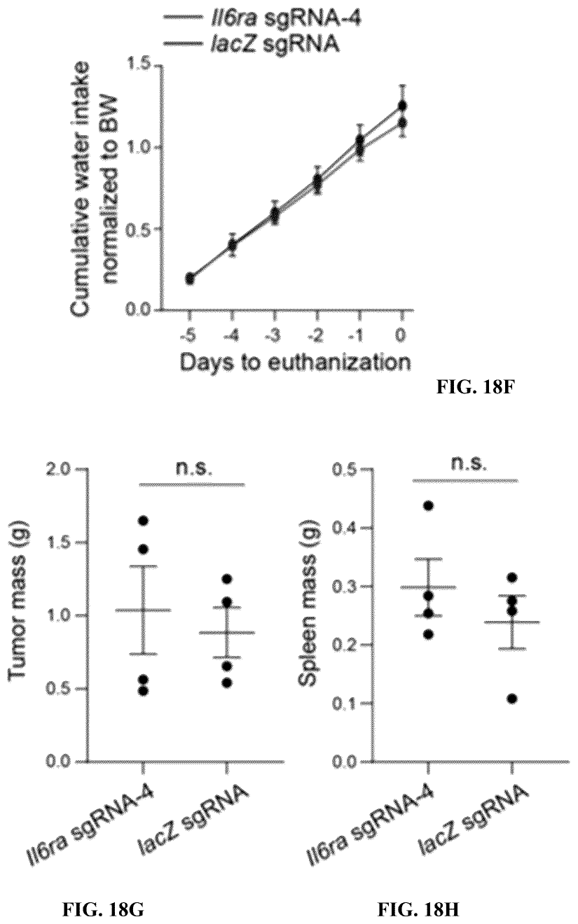

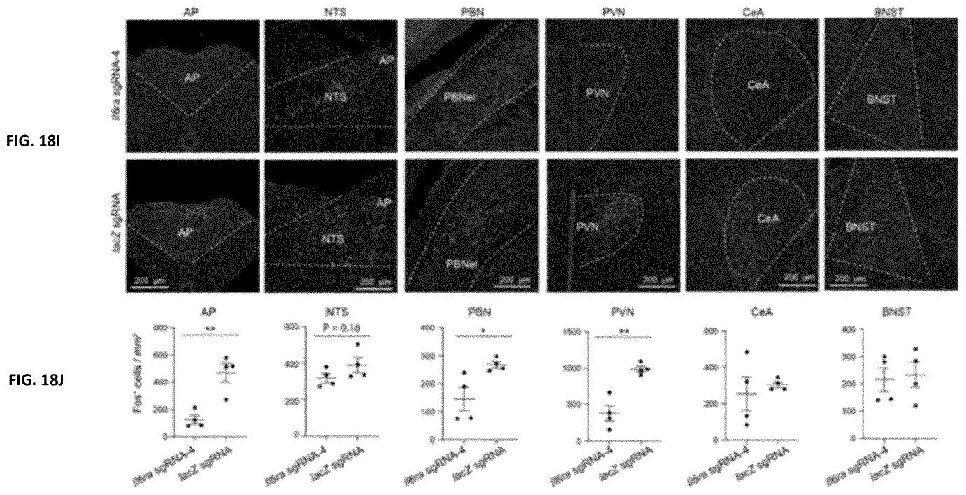

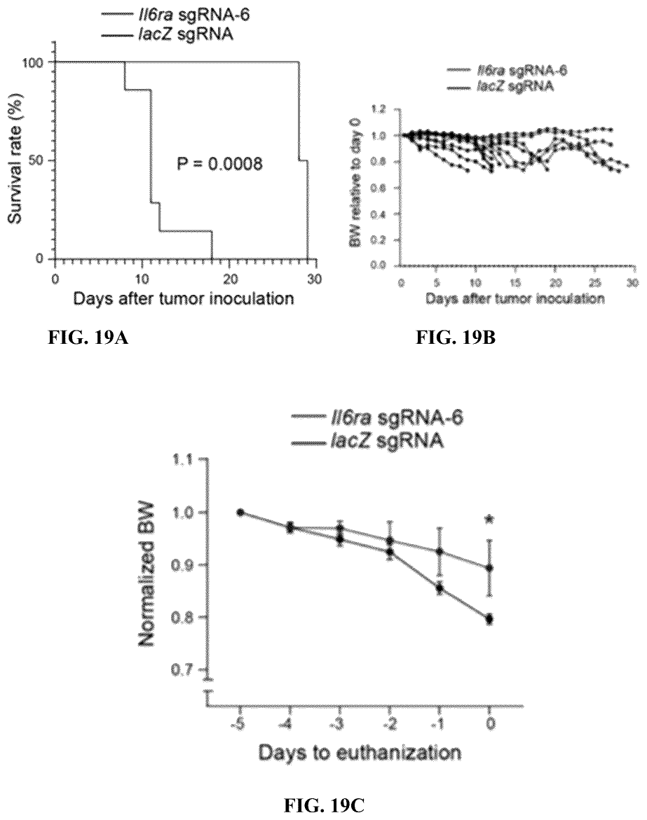

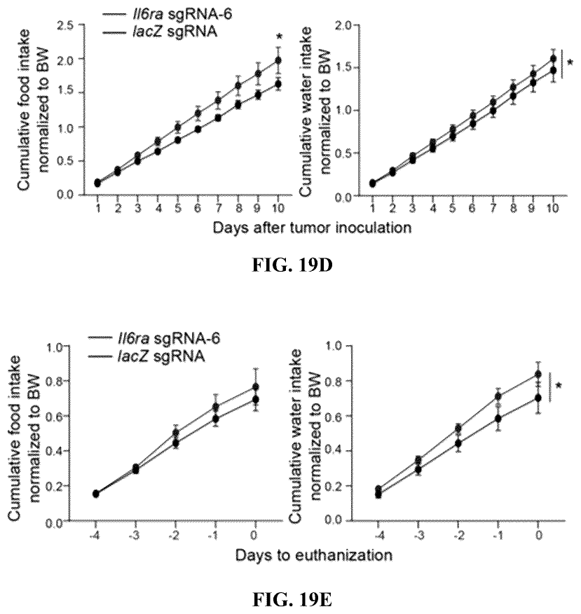

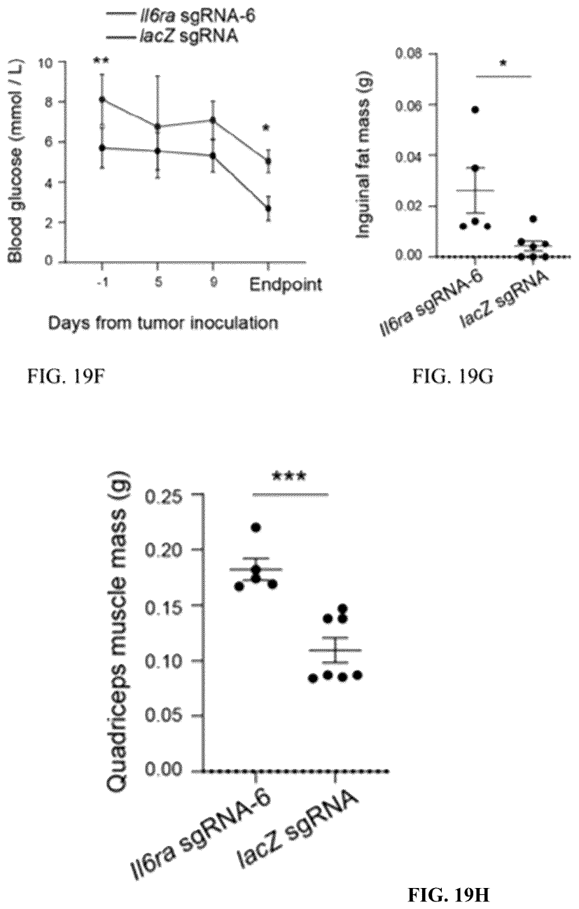

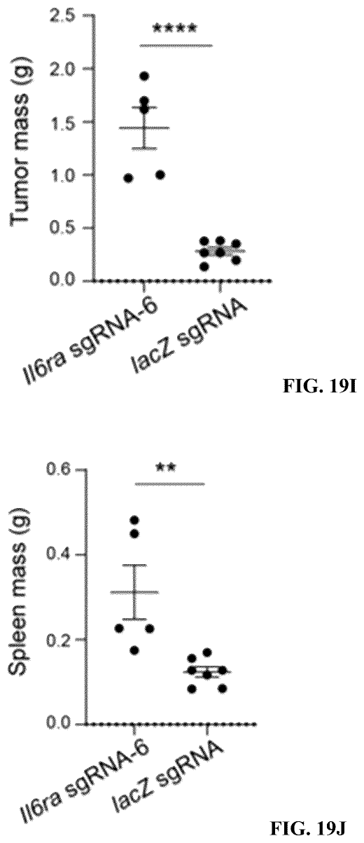

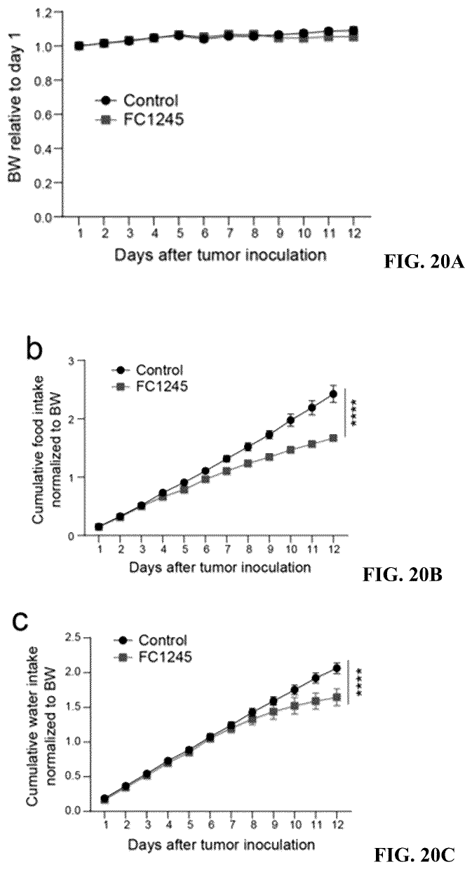

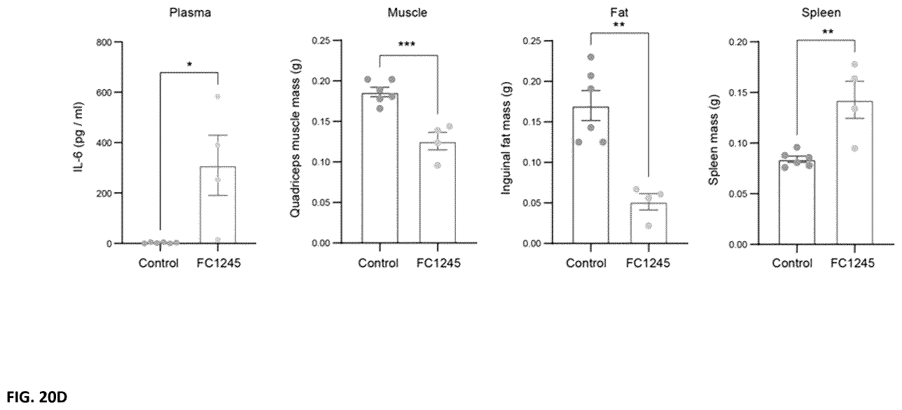

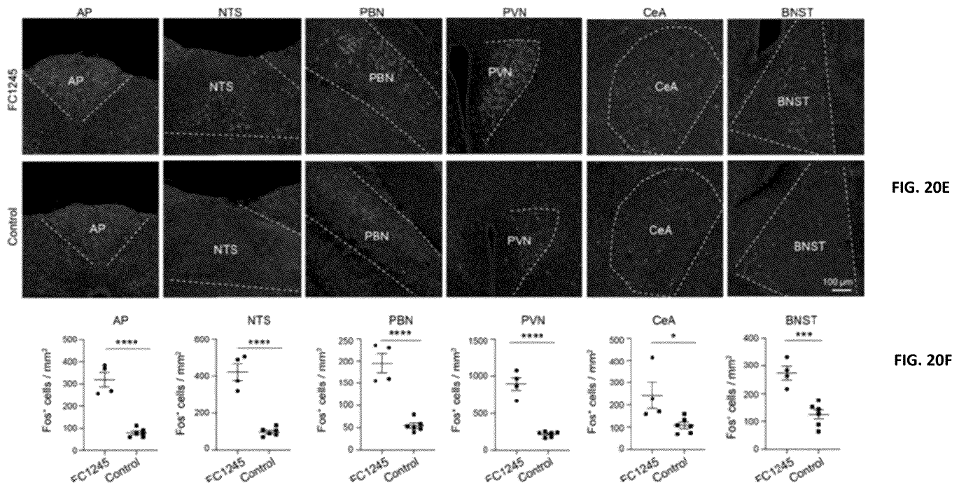

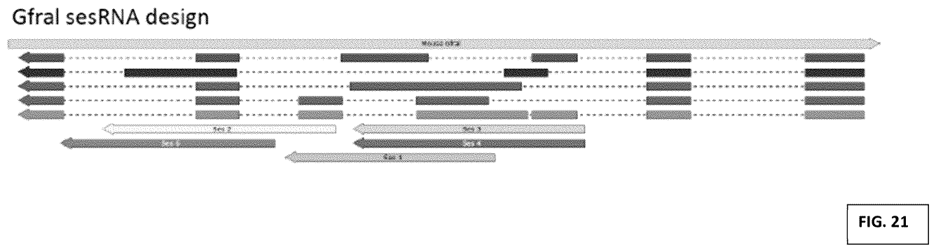

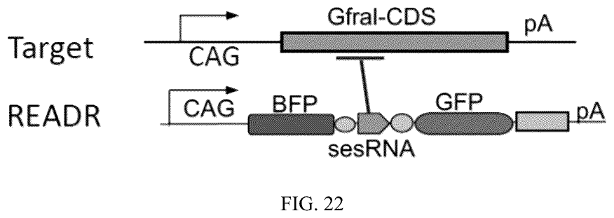

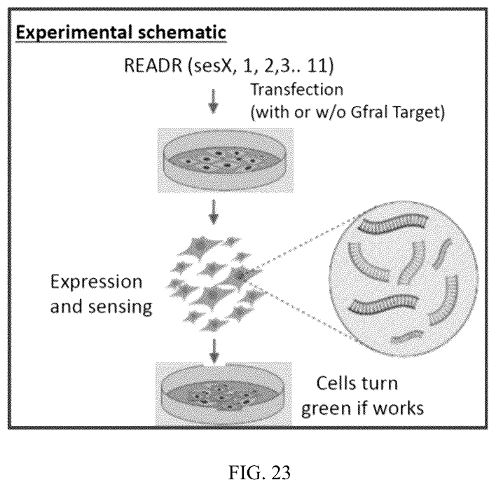

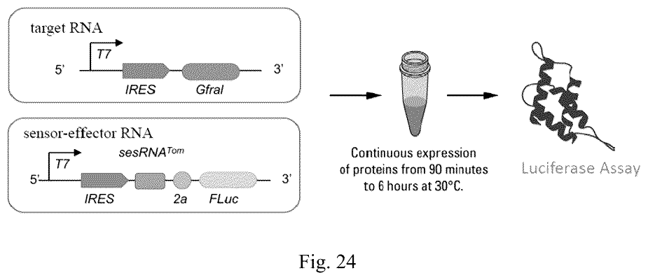

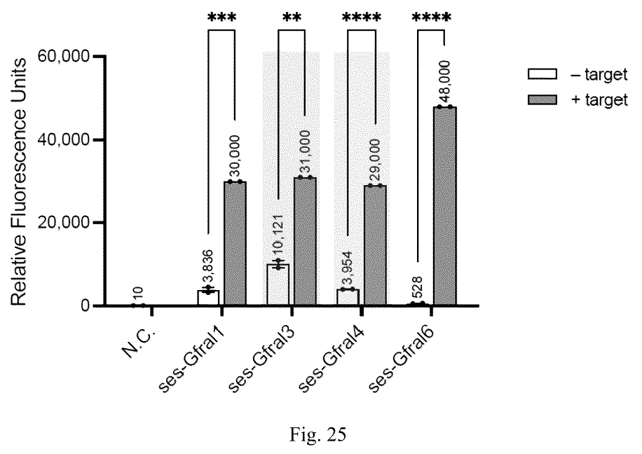

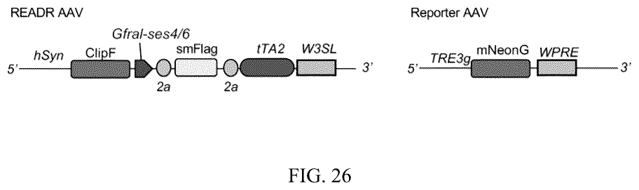

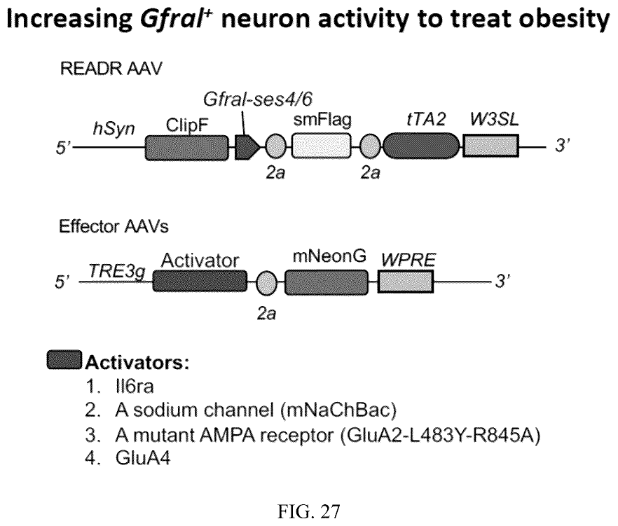

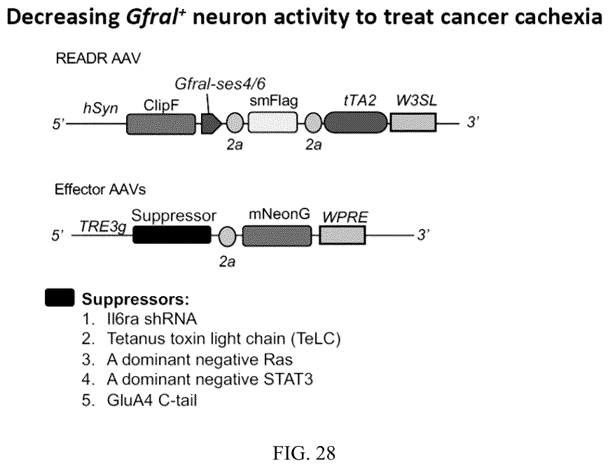

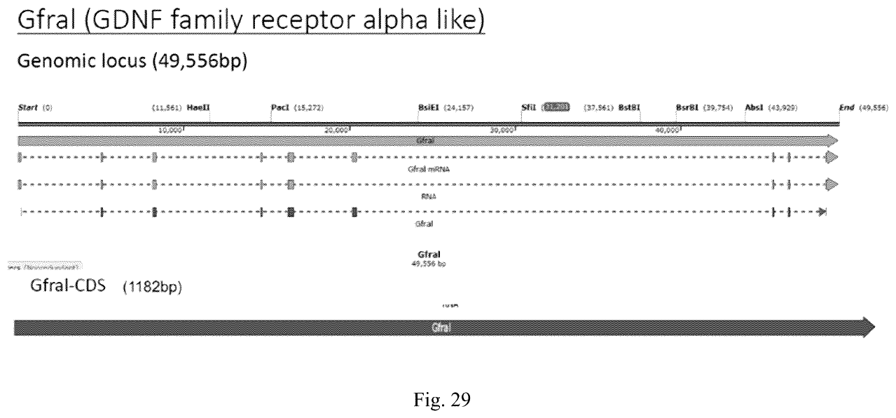

FIG. 8A indicates sesRNA^{Gfral#3} and sesRNA^{Gfral#4} comprising SEQ ID NO:s 14 and 15, respectively, show especially robust Gfral targeting efficiency and specificity for Gfral mRNA in HEK cells. Blue fluorescence is expressed from the READR construct in HEK cells with or without transfected Gfral target, while green florescence is expressed only in HEK cells with transfected Gfral target as a result of the target Gfral RNA hybridizing with the SES component of the READR construct, which triggers ADARs mediated A->I editing, converting the UAG STOP in the READR construct to a UGG Trp codon, switching on translation of the green fluorescent (GFP) effector protein.

Another experimental procedure for sesRNA^{Gfral} screening for Gfral mRNA comprising in vitro transcription in HeLa cell lysate. As illustrated below, the Gfral target is expressed with T7 promoter and an internal ribosome entry site (IRES). READR^{Gfral-GFP} encodes a readrRNA consisting of a Blue Fluorescent Protein (BFP) sequence followed by sesRNA^{Gfral} and efRNA Luc Luciferase assay was used for quantitative measurement of sesRNA^{Gfral} efficacy and specificity. See FIG. 24.

Results in FIG. 25 indicate that sesRNA^{Gfral#6} and sesRNA^{Gfral#4} show the best Gfral targeting efficiency and specificity with in vitro transcription luciferase assay.

### Step 2: Virus Packaging of Gfral-Sensor

The schematic shown in FIG. 26 of binary adeno-associated virus (AAV) vectors for targeting Gfral neurons includes a READR vector, a human synapsin (hSyn) promoter which drives transcription of ClipF (a CLIP-tag protein) followed by sesRNA (#4 or #6) and efRNA encoding an smFlag tag which is surrounded by 2A self cleving peptide, followed by tTA2 (modified tetracycline-regulated transactivator) and W3SL (a modified WPRE/polyA sequence). The Reporter vector contains a TRE3g promoter (provides very tight control of transcription) driving mNeonGreen fluorescent protein (mNeon) and a woodchuck hepatitis virus posttranscriptional regulatory element (WPRE).

FIG. 8B 300 ng of a sesRNA^{Gfral#1, #2, #3, #4, #5, #6, #7} or #8 comprising SEQ ID NO:s 4-11, respectively, function in CellReadr mediated targeting and detection of Gfral neurons in mouse area postrema, with sesRNA^{Gfral#4, 7} and 8 demonstrating especially robust staining. The relevant sequences of the Gfral sesRNAs used to target the 150 ng of mouse Gfral RNA (SEQ ID NO: 3) in vitro are listed below.

Gfral-UTR target
(SEQ ID NO: 3)
catcattttggcaaagaattggatccgccaccatgggagaactactctat
gttgttgtgtgcatggcagttacctgtggaattcttttcttggtgatgct
caagttaaggatacaaagtgaaaaaagagatccctcatccatcgaaatag
ctggaggtgtcatcattcagtgagctgcagatcacttaccaaccacatgt
ctgtgtgactaaccaatggaaaattacatttgccaataacgcaatttaag
atggatttgacaatatttagtcattatatgtaacagtgactggtacagta
atataccacaatgatcacagatctgtttttgtttttgtttttaatgtttg
agtaaatacttgttgtggtgtcataactagttgataacattttctttaaa
gacaacaggtgtcatgtaaaatgtgacaaatttgctggaagactatcaat
ccacatatcaacttctatcttatggaactaatcataattagtgtgtgcag
ttttctgaacaaggttatagttttccattaagttggtaaaattaaaatgc
taagtagaatattgagtatacttgttatttatatattcttacttagtgtc
caatcattaaacaaattggtaacattgaacatatttagttagatgactgc
ttatgaaaataagaactgacatcttacaaattttataatttaaatagtat
tgaattttactttttatttggtatgttaagattcataatatataaagcag
ctacattggttgagaaaagtcaatggttactccagtaatgatatactttg
tgaatttatttatttttgctaattaatgatcctgaatgtaatcatgatga
aataaaaaagacatacttaaaaagcttatcgataatcaacctctggatta
c
Gfral-UTR1
(SEQ ID NO: 4)
gacgtggaggagaaccctggacctgaattctgcaaagctgatatgagtac
cagtactggtatttataactctagcatgaagacttccttatattgcaatt
tacctcttcattctaatttattgctctttgattttgtgtAgttgcattca
ctctcctgccaggtcaatgtgactaaattaattccaatacaaacagaaaa
ggtcttaagtaggattgaatggtacttttgttttgtgctttgttgctctg
tcaggctggattttgctcaccaaggcgcgccagagggcagaggaagtctt
cta
Gfral-UTR2
(SEQ ID NO: 5)
ggaggagaaccctggacctgaattctttaagtatgtcttttttatttcat
catgcttacattcaggatcattaattagcaaaaataaagaaattcacaaa
gtatatcattactAgagtaaccattgccttttctcaaccaatgtagctgc
tttatatattatgaatcttaacataccaaataaaaagaaaattcaatact
atttaaattataaaatttgtaagatgtcagttcttattttcataagcagt
catctaactaaatatgttcaatgttaccaatttgttggcgcgccagaggg
cagaggaagtcttcta
Gfral-UTR3
(SEQ ID NO: 6)
ggaggagaaccctggacctgaattcaaagtaaaattcaatactattgaaa
ttataaaatttgtaagatgtcagttcttattttcatacgcagtcatcgaa
ctaaatatgttcaatgttaccaatttgtttaatgattAgacactaagtaa
gaatatatacataacaagtatactcaatattctacttagcattttaattt
taccaacttactggaaaactataaccttgttcagaaaactgcacacacta
attatcattagttccataagaggcgcgccagagggcagaggaagtcttct
a
Gfral-UTR4
(SEQ ID NO: 7)
ggaggagaaccctggacctgaattcctaaatatgttcaatgttaccaatt
tgtttaatgattggacactaagtaagaatatacaaataacaagtatactc
aatattctacttagcattttaattttaccaactgaatAgaaaactataac
cttgttcagaaaactgcacacactaattatgattagttccataagataca
agttgatatgtggattgatagtcttccagcaaatttgtcacattttacat
gacacctgttgtcttggcgcgccagagggcagaggaagtcttcta
Gfral-UTR5
(SEQ ID NO: 8)
ggaggagaaccctggacctgaattcatttgtcacattttacatgacacct
gttgtctttacagaaaatgttatcaactagttatgacaccacaacaagta
tttactcaaacattaaaaacaaaaacaaaaacagatctgcgatcattgtA
gtatattactgtaccagtcactgttacatataatgactacatattgtcaa
atccatcttacattgcgttattggcaaatgtaattttccattggttagtc
acacagacatgtggttggggcgcgccagagggcagaggaagtcttcta
Gfral-UTR6
(SEQ ID NO: 9)
ggaggagaaccctggacctgaattccaaacattaaaaacaaaaacaaaaa
cagatctgtgctcattgtggtatattactgtaccagtcactgttacatat
aatgacgaaatattgtcaaatccatcttacattgcgttattggcaaatgt
aattttccattAgttagtcacacagacatgtggttggtacgtgatctgca
gctcactgaatgatgacacctccagctatttcgatggatgtgggatctct
tttttcactttgtatggcgcgccagagggcagaggaagtcttcta
Gfral-UTR7
(SEQ ID NO: 10)
ggaggagaaccctggacctgaattcaaacaaaaacaaaaacagatctgtg
atcattgtggtatattactgtaccagtcactgttacatataatgactaaa
tattgtcaaatccatcttaaattgcgttattAgcaaatgtacattttcca
ttAgttagtcacacagacatgtggttggtacgtgatctgcagctcactga
atgatgacacctccagctatttcgatggatgtgggatctcttttttcact
ttgtatggcgcgccagagggcagaggaagtcttcta
Gfral-UTR8
(SEQ ID NO: 11)
ggaggagaaccctggacctgaattctcaaacattaaaaacaaaaacaaaa
acagatctgtgatcattgtggtatattactgtaccagtcactgttacata
tactgcctaaatattgtcaaatccatcttaaattgcgttattggcaaatg
taattttccattggttagtcacacagacatgtAgttggtaagtgatctgc
agctcactgaagatgacacctccagctatttcgatggatgtgggatctct
tttttcactttgtatggcgcgccagagggcagaggaagtcttcta

Step 3: Design and Testing Gfral sesRNA-Based Effectors for Neuronal Manipulation

A strategy to increase the activity of Gfral-expressing (Gfral+) neurons in the area postrema for the treatment of obesity is presented in FIG. 27.

A strategy to decrease the activity of Gfral+ neurons in the area postrema for the treatment of cancer cachexia is illustrated in FIG. 28.

### Working Example II—The area postrema of mice and IL-6

The area postrema of mice senses circulating IL-6.

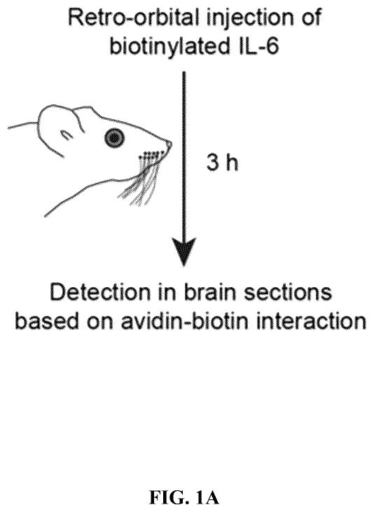

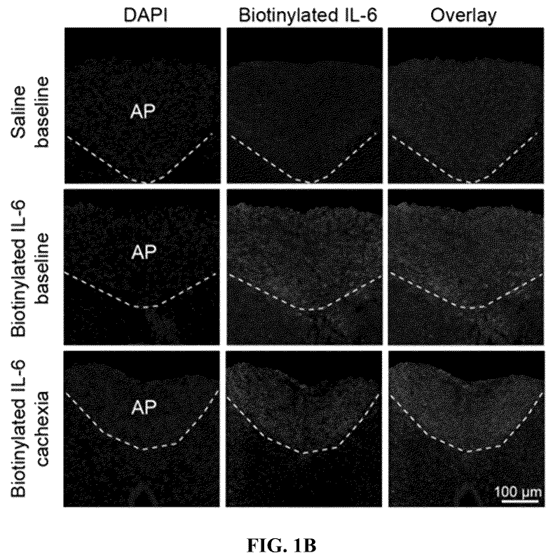

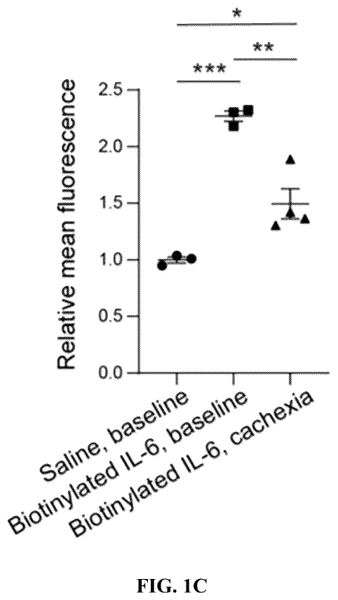

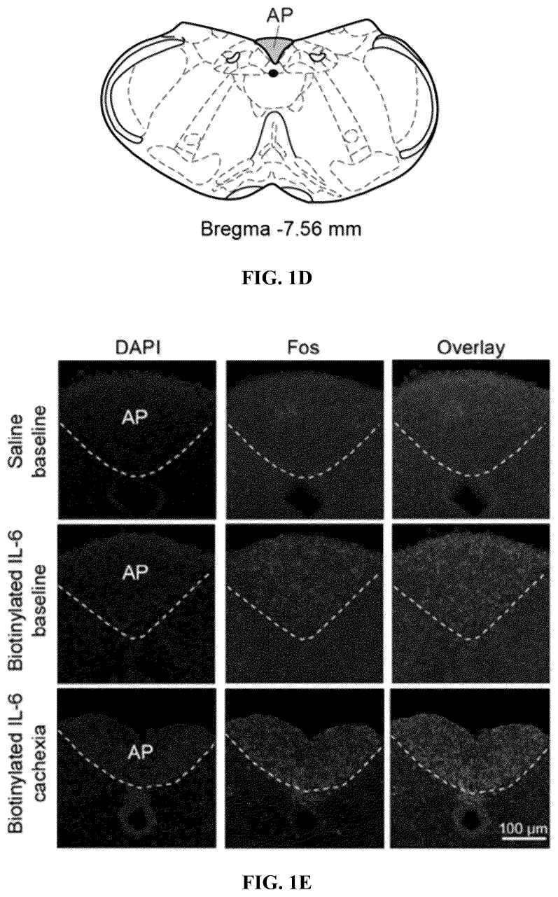

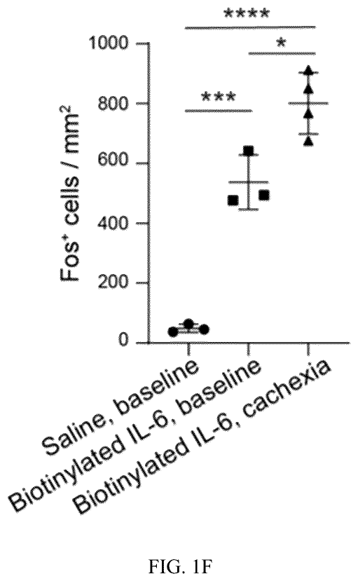

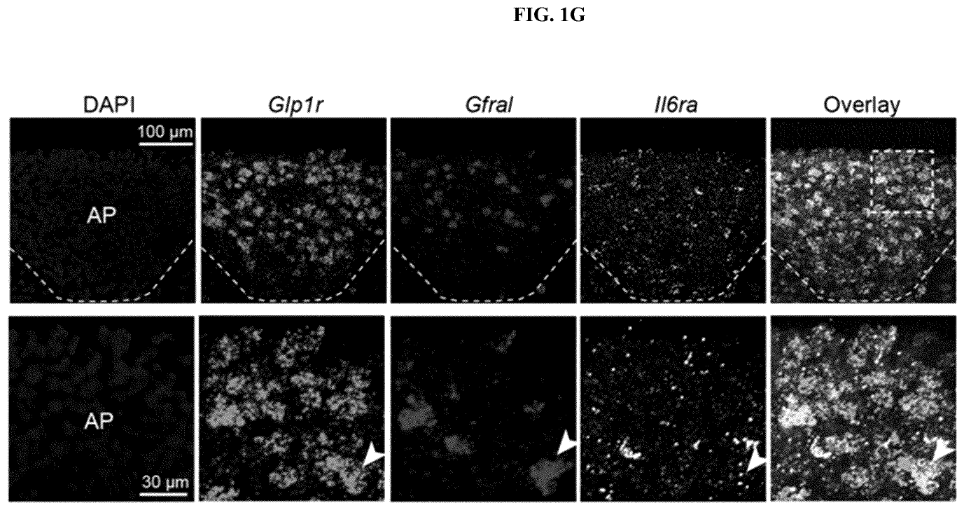

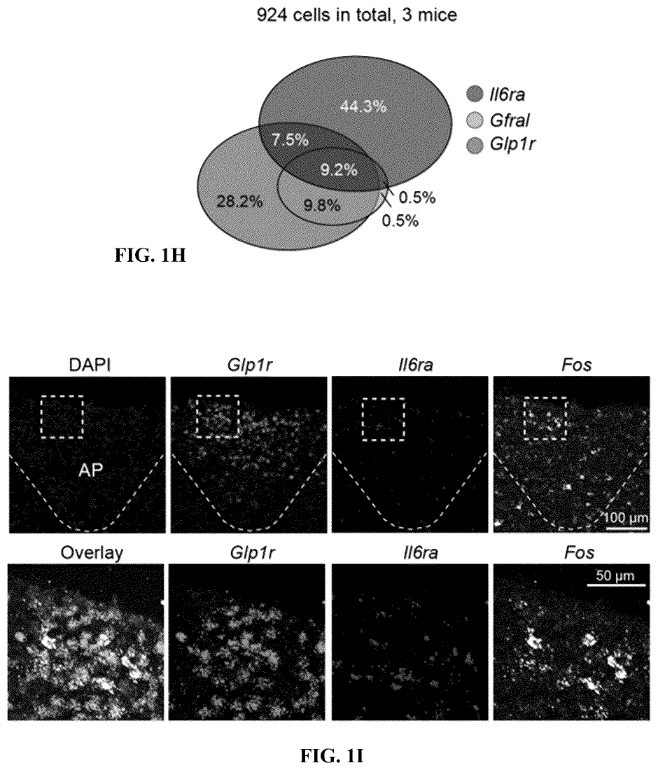

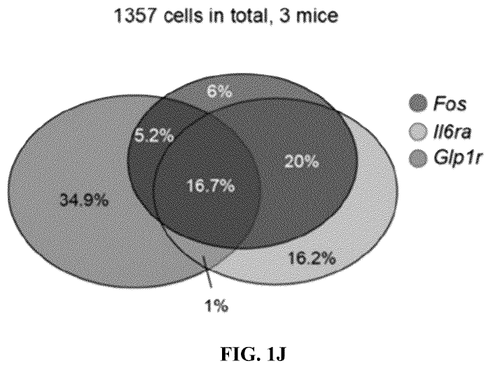

In order to determine if circulating IL-6 can enter the brain at the baseline state and a cachectic state, biotinylated IL-6 was administered to the venous sinus of healthy mice and mice that had developed cancer cachexia through retro-orbital injection. Three hours later, we prepared brain sections from these mice in which the presence of the exogenous IL-6 was examined on the basis of avidin-biotin interactions. In the entire brain of both healthy mice and cachectic mice, we detected the peripherally administered IL-6 only in the area postrema (AP) (FIG. 1a-d; Data of FIG. 9), which is a circumventricular organ located outside of the BBB and has been implicated in nausea and vomiting response to emetic agents entering the circulation⁴⁷⁻⁵⁰. We did not detect the peripherally administered IL-6 in another circumventricular organ, the median eminence (ME)^{51,52} (Data of FIG. 9).

Immunohistochemistry in the AP of healthy mice revealed that the retro-orbital injection of IL-6 administration markedly increased the expression of Fos (FIG. 1e, f), an immediate early gene product linked to recent neuronal activation^{53,54}. Interestingly, AP Fos expression was further increased in mice displaying a cachectic state (FIG. 1e, f). The IL-6 administration also increased Fos expression in areas interconnected with the AP, including the nucleus tractus solitarii (NTS), parabrachial nucleus (PBN), paraventricular nucleus of the hypothalamus (PVN), central amygdala (CeA), bed nucleus of the stria terminalis (BNST), and arcuate hypothalamic nucleus (ARH) (Data of FIG. 18). The dorsal part of lateral septum (LS) and ME, on the other hand, showed no obvious increase in Fos expression (Data of FIG. 10).

Single molecule fluorescent in situ hybridization (smFISH) revealed that the Il6ra-expressing (Il6ra⁺) cells in the AP partially overlapped with glucagon-like peptide 1 receptor-expressing (Glp1r⁺) neurons (FIG. 1g-j; Data of FIG. 11), the major excitatory neuronal type in the AP50. About 17-18% of all the detected AP cells (which likely included glia cells) expressed both Il6ra and Glp1r (FIG. 1h & j). These Il6ra⁺ cells also partially overlapped with Gfral-expressing (Gfral ⁺) neurons (FIG. 1g, h; Data of FIG. 11), a subpopulation of Glp1r⁺ neurons in the AP that have been implicated in nausea and cancer-associated cachexia^{28,29,50} The adjacent NTS also contains I/6ra cells and scattered Gfral ⁺ neurons that do not express Glp1r (Data FIG. 11). Interestingly, intravenous IL-6 administration induced Fos expression mainly in the Il6ra⁺ cells in the AP, and these cells partially overlapped with the Glp1r⁺ neurons (FIG. 1i, j). These results demonstrate that increased IL-6 in circulation is readily “sensed” by the AP, where it leads to neuronal activation within hours.

### Part B Tumor Causes AP Hyperactivity

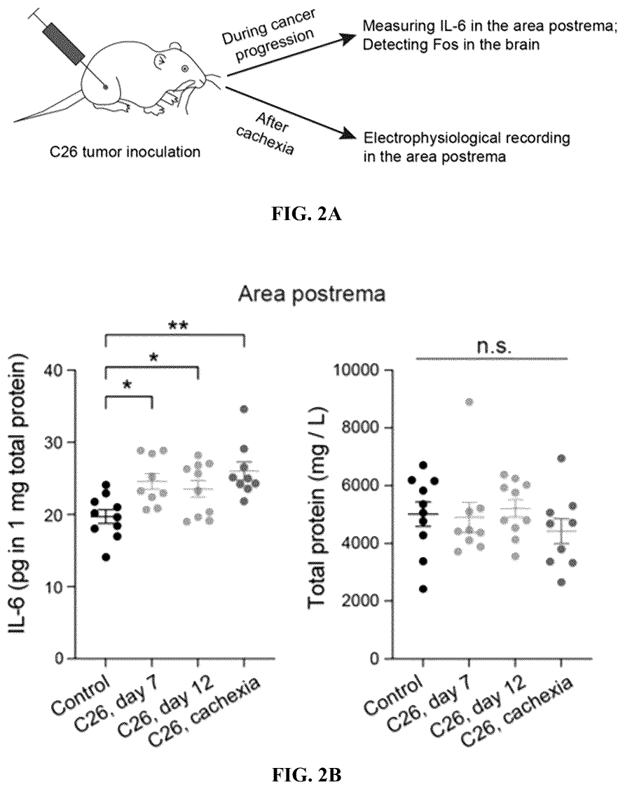

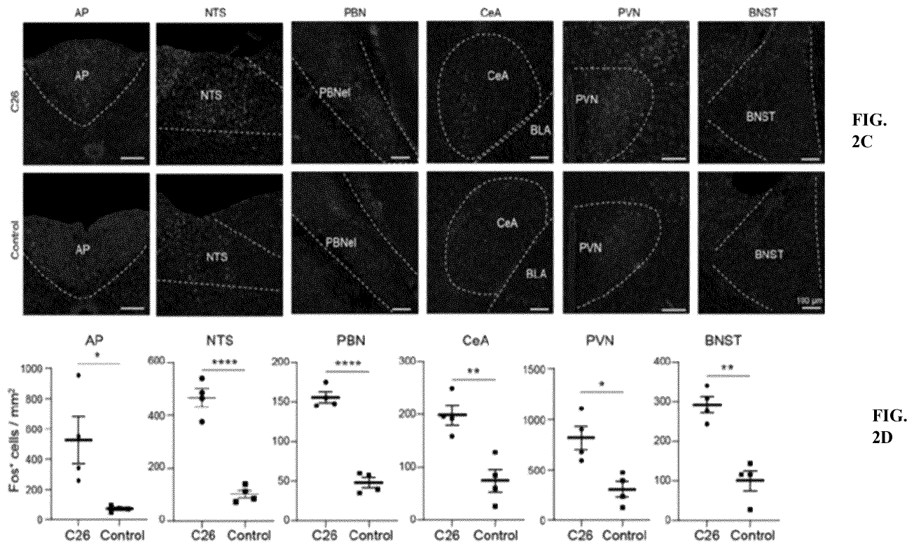

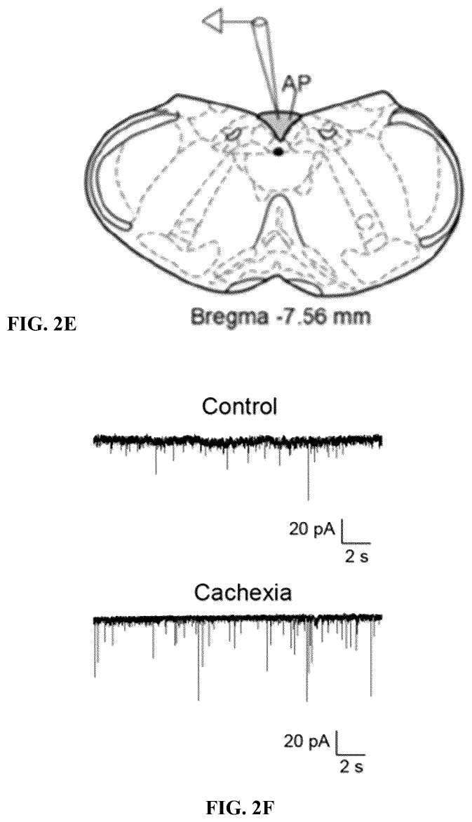

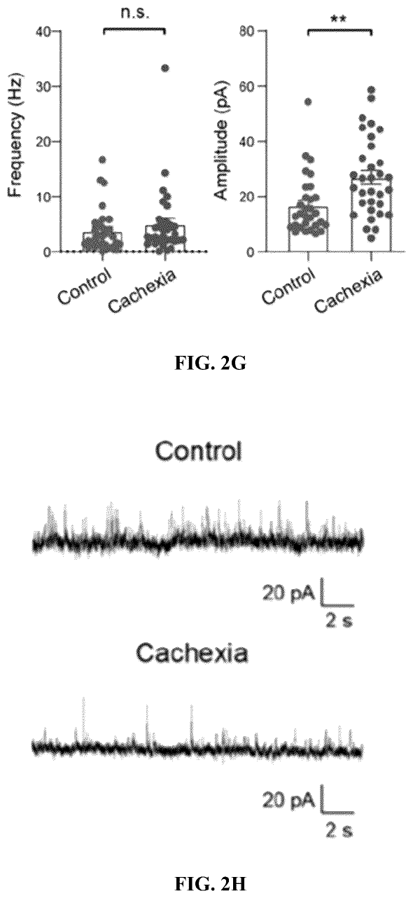

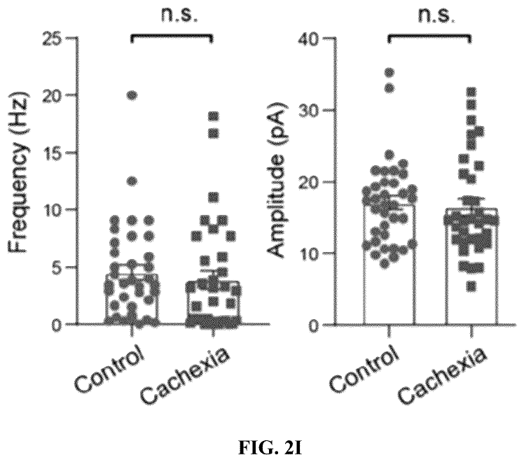

To investigate whether cancer, which is known to increase circulating IL-6^{1-5,15}, affects AP neurons, we used mice inoculated with the C26 adenocarcinoma (FIG. 2a, Methods). Mice in this model show persistent increase in blood IL-6 levels, followed by robust cachectic phenotypes, including anorexia and dramatic bodyweight loss^{15,18,21-23}. We first measured IL-6 levels in the AP at different timepoints in this model. Notably, IL-6 was increased in the AP on day 7 following tumor inoculation, as well as after the animals had developed cachexia (FIG. 2b). In contrast, in the ME, IL-6 levels didn't increase until after the onset of cachexia, while in the cortex IL-6 levels didn't increase throughout the different stages (Data of FIG. 12). These results are consistent with the above observations that the AP is more accessible to circulating IL-6.

We then examined Fos expression in the brain in this model before the onset of cachexia (11 days after tumor inoculation). The number of Fos+ cells in the AP was markedly increased in the tumor-bearing mice compared with control mice (FIG. 2c, d). Interestingly, the NTS, PBN, PVN, BNST, and CeA-which are structures previously implicated in cancer cachexia^{17,27-29}—also showed tumor-induced increase in Fos+ cells. Given these structures are interconnected⁵⁵⁻⁵⁸ and receive monosynaptic or di-synaptic inputs from the AP50,59, these results suggest that cancer progression leads to increased activities in a network of brain areas encompassing the AP. Next, we tested whether cachexia is associated with lasting functional changes in AP neurons. We prepared acute brain slices from healthy control mice as well as the tumor-bearing mice already showing cachectic phenotypes, and recorded synaptic responses from AP neurons (FIG. 2e). We found that the amplitude of miniature excitatory postsynaptic currents (EPSCs) was markedly increased, while the frequency of the EPSCs was unchanged in cachectic mice relative to control mice (FIG. 2f, g). In contrast, there was no difference in the inhibitory postsynaptic currents (IPSCs) between cachectic mice and control mice (FIG. 2h, i). These results demonstrate that cancer-associated cachexia is accompanied by a potentiation of excitatory synaptic transmission onto AP neurons, which may lead to hyperactivity in these neurons.

### Part C Neutralizing IL-6 in the Brain Prevents Cancer-Associated Cachexia and AP Hyperactivity

We reasoned that increased circulating IL-6 during cancer progression readily enters the AP and results in AP neuron activation-like the intravenously administered exogenous IL-6 (FIG. 1)—leading to hyperactivity in these neurons, which contributes to the development of cachexia.

As a first step to test this hypothesis, we neutralized IL-6 in the brain by intracerebroventricular (i.e.v.) infusion of an antibody against IL-6, which was achieved using an implanted miniature pump (Methods). Continuous infusion of the IL-6 antibody, or an isotype control antibody, was initiated at 10 or 12 days after tumor inoculation (Data of FIG. 13a, 13b), a stage when AP neurons show elevated activity (FIG. 2c-d) but cachexia has not yet started. Remarkably, compared with the control antibody, the IL-6 antibody prevented the cachectic phenotypes in almost all the mice, prolonging lifespans (Data of FIG. 13c), reducing bodyweight loss and tissue loss (Data of FIG. 13d, 13e; Data of FIG. 14a, 14b), increasing food and water intake (Data FIG. 13f), and increasing blood glucose levels (Data of FIG. 14c). Moreover, IL-6 antibody infusion reduced Fos expression in the AP, PBN, PVN, BNST, and, to a lesser extent, CeA (Data of FIG. 13g, 13h). The IL-6 antibody did not change IL-6 levels in the plasma, but had a trend to decrease IL-6 levels in the cerebrospinal fluid (P=0.067; Data of FIG. 14d). As expected, i.e.v. infusion of IL-6 antibodies did not stop tumor from growing (Data of FIG. 14e, 14f).

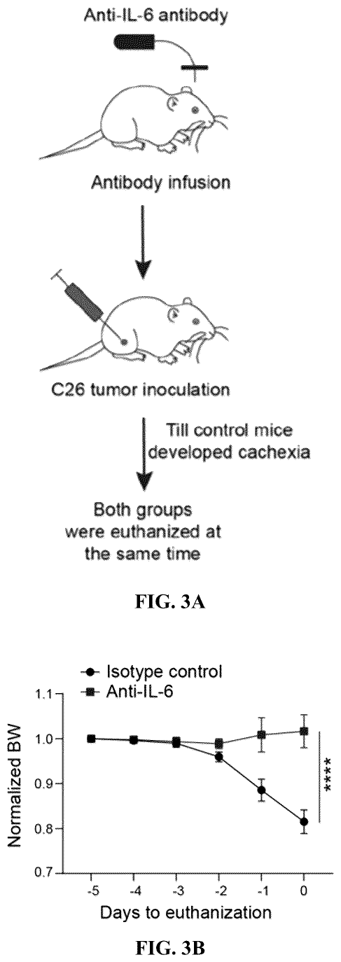

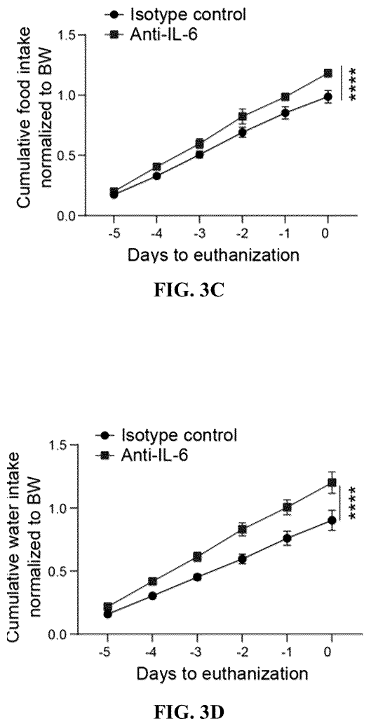

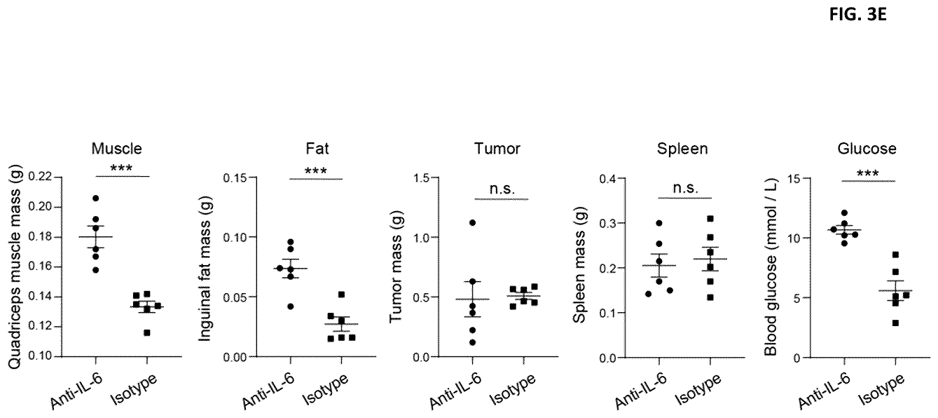

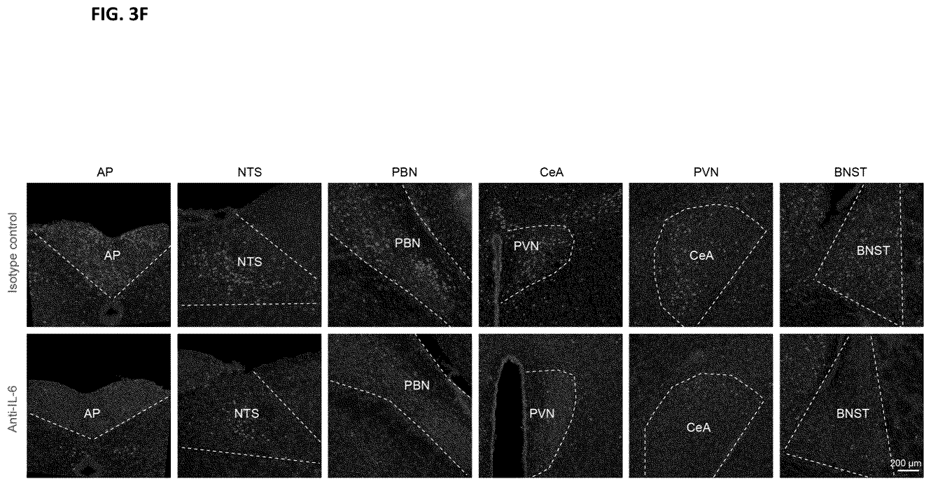

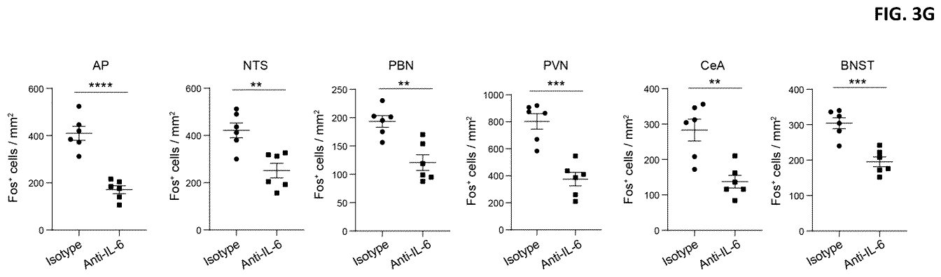

We next tested whether i.e.v. infusion of IL-6 antibodies before tumor inoculation can also prevent cachexia (FIG. 3a-g). We found that the pretreatment could also prevent cachexia, with animals in the IL-6 antibody group showing higher bodyweight, food intake, and water intake than those in the control group (FIG. 3b-d). The pretreatment also prevented tissue wasting, increased blood glucose levels, and reduced hyperactivity in the AP and its connected brain areas (FIG. 3e-g). Of note, unlike the experiment described above (Data of FIG. 13), in this experiment we euthanized both groups of mice at the same timepoint, therefore the two groups had similar tumor and spleen mass (FIG. 3e). Together, these experiments demonstrate that reducing IL-6 levels in the brain during cancer progression effectively prevents cachexia, and also dampens the cancer-associated hyperactivity in the AP network.

### Part D Suppression of Il6Ra Expression in AP Neurons Attenuates Cancer-Associated Cachexia in the C26 Model

To investigate whether AP neurons mediate the functions of IL-6 in the development of cancer-associated cachexia, we sought to suppress Il6ra, the gene encoding IL-6Rα, in these neurons using a recently developed CRISPR/dCas9 interference system. This system consists of dCas9-KRAB-MeCP2 [a fusion protein including the nuclease-dead Cas9 (dCas9), a Krüppel-associated box (KRAB) repressor domain, and the methyl-CpG binding protein 2 (MeCP2)] for transcriptional repression, and a CRISPR sgRNA (single guide RNA) for targeting the promoter region of genes of interest^{60,61}. We designed and identified two sgRNAs, Il6ra-sgRNA-4 and -6, that resulted in suppression of Il6ra transcription when co-expressed with dCas9-KRAB-MeCP2 in in vitro screening (Data of FIG. 15).

We injected the AP with a lentivirus expressing dCas9-KRAB-MeCP2 in neurons (lenti-SYN-FLAG-dCas9-KRAB-MeCP2), together with a lentivirus expressing Il6ra-sgRNA-4 (lenti-U6-Il6ra sgRNA-4/EF1α-mCherry), or a sgRNA targeting the bacterial gene lacZ as a control (lenti-U6-lacZ sgRNA/EF1α-mCherry)⁶⁰ (Data of FIG. 16). Three weeks later, we collected the brains and prepared sections containing the AP for smFISH, which revealed that most of the mCherry signals colocalized with the NeuN signals (Data of FIG. 16a, 16b), indicating that viral expression was specific to neurons. Furthermore, the Il6ra-sgRNA-4 group had reduced expression in AP neurons, including the Gfral + neurons (Data of FIG. 16c, 16d), showing the efficiency of this approach.

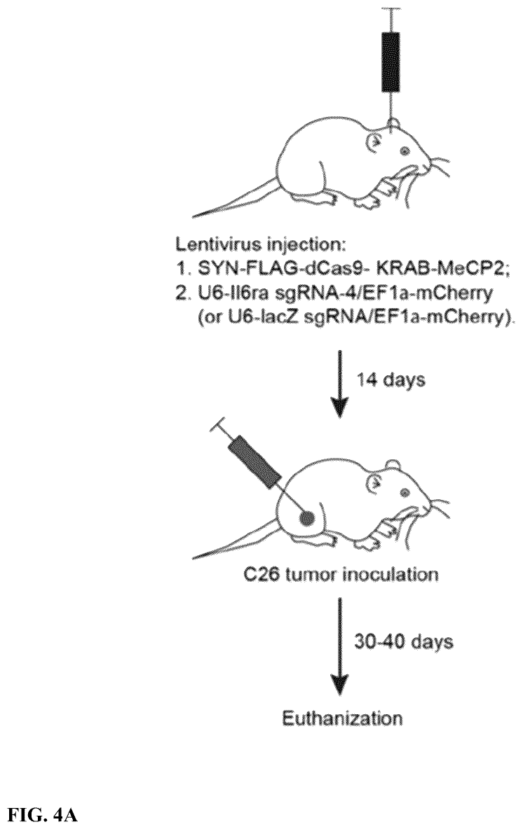

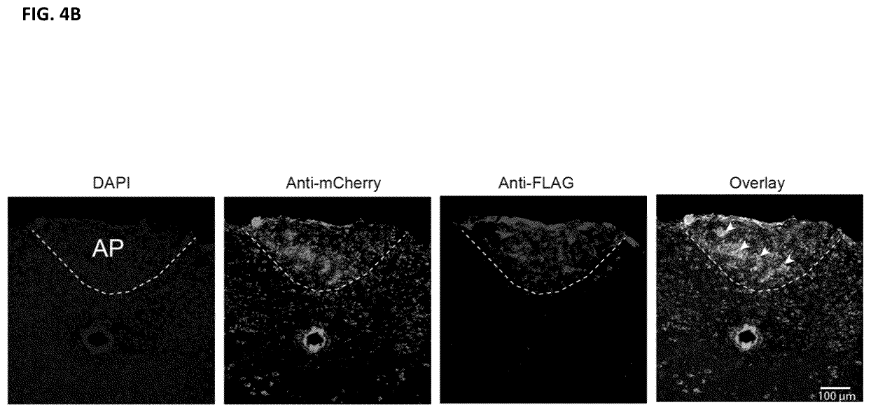

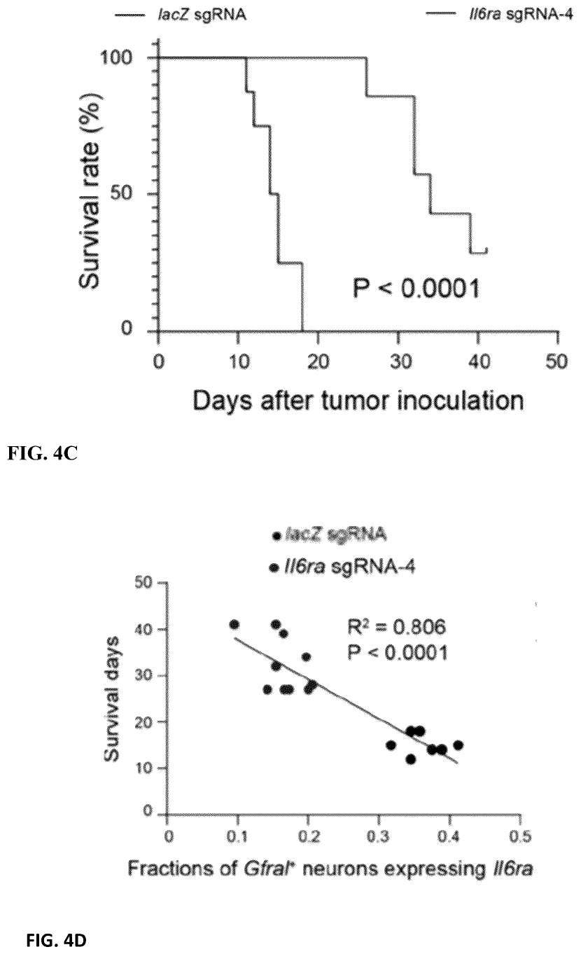

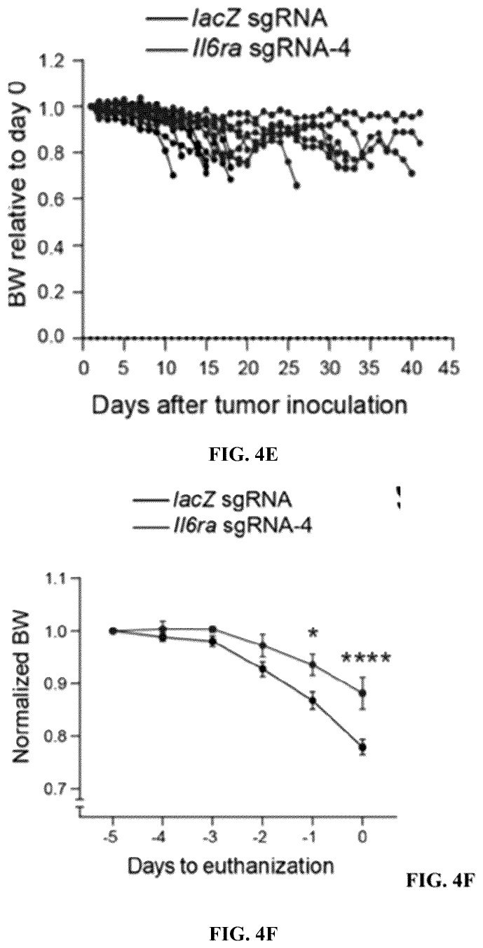

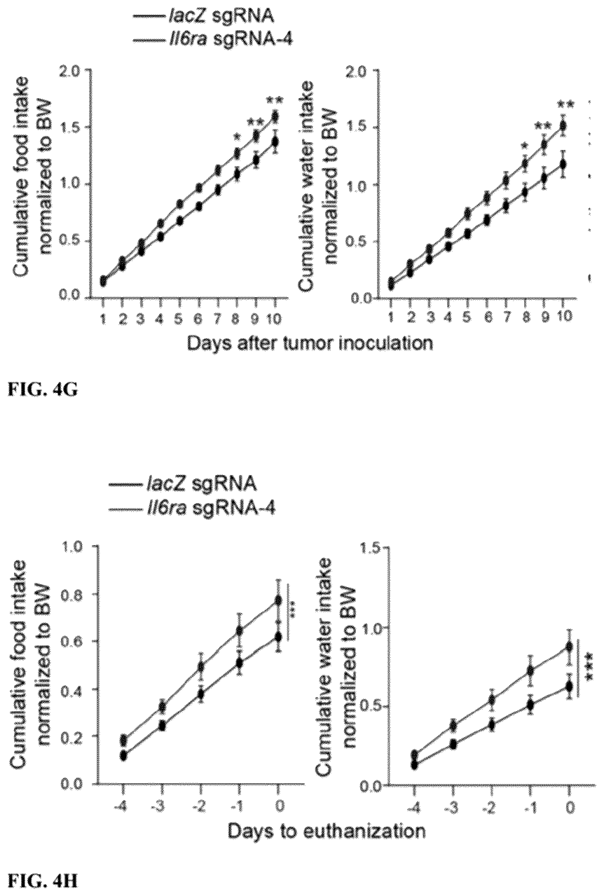

We then examined whether this approach could prevent cachexia. We injected the viruses into the AP, and two weeks later, inoculated the mice with the C26 tumor (FIG. 4a, b). Notably, the Il6ra-sgRNA-4 group had markedly increased lifespans (FIG. 4c), an effect that was inversely correlated with the percentage of Gfral + neurons that had detectable Il6ra expression (FIG. 4d). The Il6ra-sgRNA-4 group also had reduced bodyweight loss (FIG. 4e, f), increased food and water intake (FIG. 4g, h), increased blood glucose levels (Data of FIG. 17a), and had a tendency to reduce tissue loss (Data of FIG. 17b, 17c). At the endpoint of the experiment, mice in the Il6ra-sgRNA-4 group had larger tumor and spleen compared with mice in the control group (Data of FIG. 17d, 17e), presumably because of the increase in lifespan in the former group.

In a separate experiment, the Il6ra-sgRNA-4 mice and the lacZ-sgRNA control mice were euthanized on the same day, as soon as the latter had developed cachexia (Data of FIG. 18a). This design ensured that the two groups had tumor for the same duration and thus had comparable tumor and spleen mass (Data of FIG. 18g, 18h). Consistent with the above observations, the Il6ra-sgRNA-4 group had reduced bodyweight loss and tissue loss (Data of FIG. 18b-d), even though these mice had similar food and water intake to the control mice before the termination of this experiment (Data of FIG. 18e, 18f). Interestingly, the Il6ra-sgRNA-4 mice had reduced Fos expression compared with the lacZ sgRNA mice in the AP, PBN, and PVN (Data of FIG. 18i, 18j), suggesting that suppression of Il6ra expression in AP neurons lowers the hyperactivity in the AP network.

Additional experiments showed that the other sgRNA, the Il6ra sgRNA-6, had similar effects to those of Il6ra sgRNA-4 Data of FIG. 19). Together, these results indicate that the IL-6Ra on AP neurons, especially that on Gfral+ neurons, is a critical mediator of IL-6 function in the development of cancer-associated cachexia in the C26 model.

Part E Suppression of Il6ra expression in AP neurons attenuates cancer-associated cachexia in a PDAC model

We further tested whether suppressing Il6ra in AP neurons could prevent cachexia in an orthotopic tumor model, which is a well characterized pancreatic ductal adenocarcinoma (PDAC) model based on the FC1245 clonogenic cell line^{62,63}. Mice in this model showed decreased food and water intake, although their bodyweight didn't decrease (Data of FIG. 20a-c).

These phenotypes are consistent with previous findings^{62,63}, which suggest that the unchanged bodyweight is due to abdominal ascites and third spacing edema. The tumor bearing animals also showed other features cachexia, including increased IL-6 levels in the plasma and reduced muscle weight and fat weight (Data of FIG. 20d). Interestingly, the tumor-bearing animals also showed increased Fos expression in the AP network (Data of FIG. 20e, 20f).

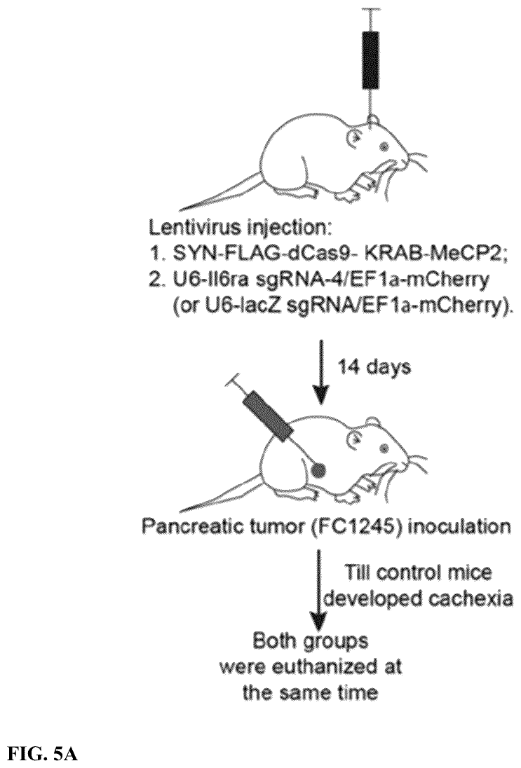

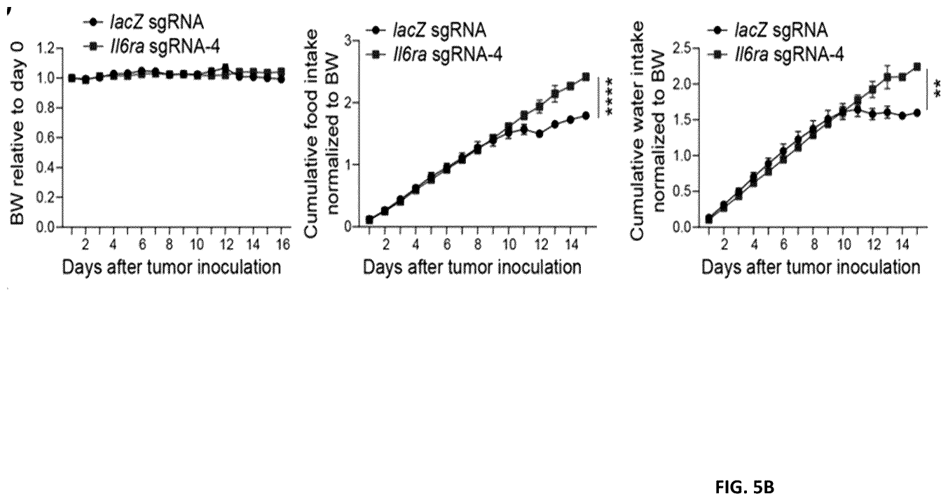

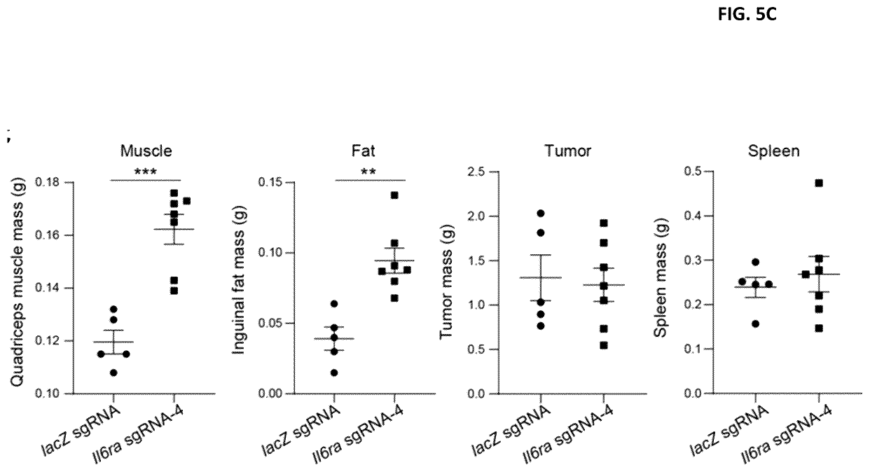

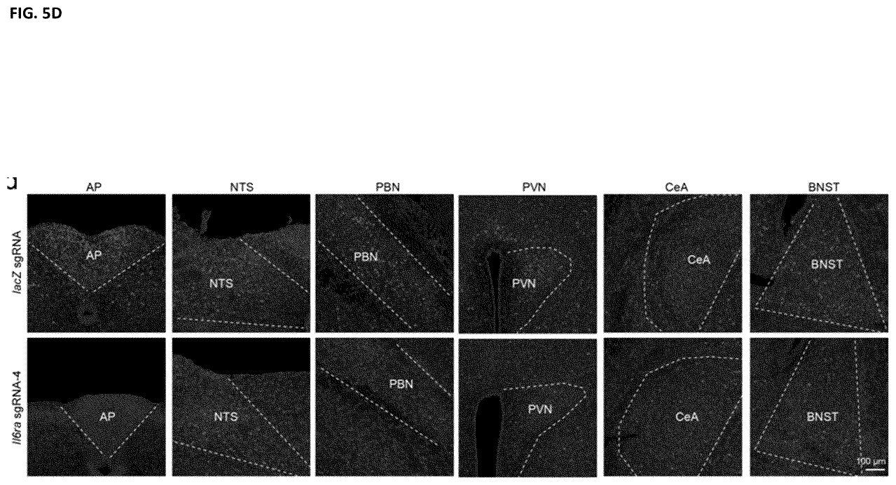

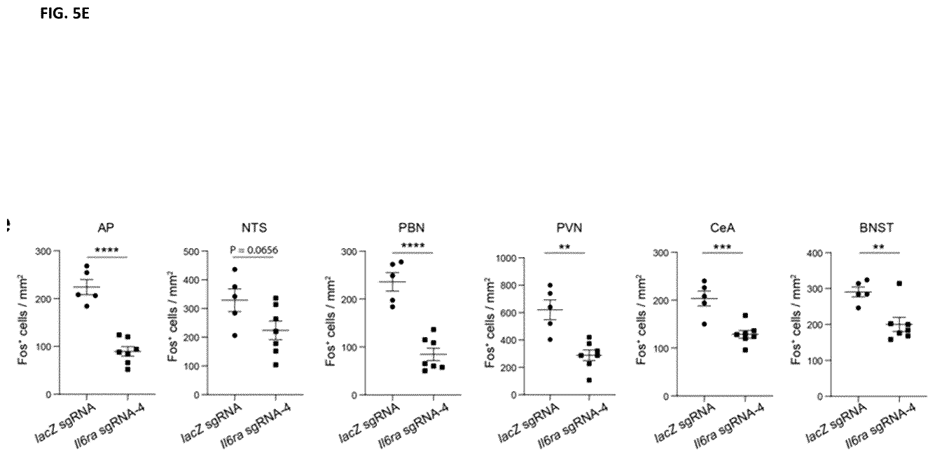

As described above, we again delivered the CRISPR/dCas9 system containing I/6ra-sgRNA-4 or the control lacZ-sgRNA to AP neurons (FIG. 5a). Two weeks after the viral injection, we inoculated FC1245 cells in the pancreas of these mice. We found that the Il6ra-sgRNA-4 mice had improved food and water intake, decreased tissue wasting compared with the control mice (FIG. 5b, 5c). The Il6ra-sgRNA-4 mice also had reduced Fos expression in the AP and its interconnected areas (FIG. 5d, 5e). Thus, suppressing Il6ra in AP neurons also prevents cachexia and reduces AP network hyperactivity in the PDAC model.

### Part F Inhibition of Gfral+ Neurons Attenuates Anorexia in the LLC Model

Given the result that the survival of mice in the C26 model was correlated with the suppression of Il6ra in Gfral+ neurons in the AP (FIG. 4d), and previous findings that Gfral and its ligand GDF-15 (Growth/differentiation factor 15) are involved in cancer-associated cachexia^{28,29}, we reasoned that the activity of Gfral . AP neurons contributes to the development of this syndrome. To test this hypothesis, we sought to selectively manipulate these neurons using the Gfral-p2a-Cre mice⁵⁰ in combination with a Cre-dependent viral approach. As the C26 model necessitates the use of Balb/c or CD2F1 mice⁶⁴, it is incompatible with the Gfral-p2a-Cre mice which have a C57BL/6 genetic background. Therefore, for this experiment, we used the implantable Lewis lung carcinoma (LLC) model, another established murine cancer model exhibiting features of cachexia, albeit milder than those of the C26 model^{18,23,27}.

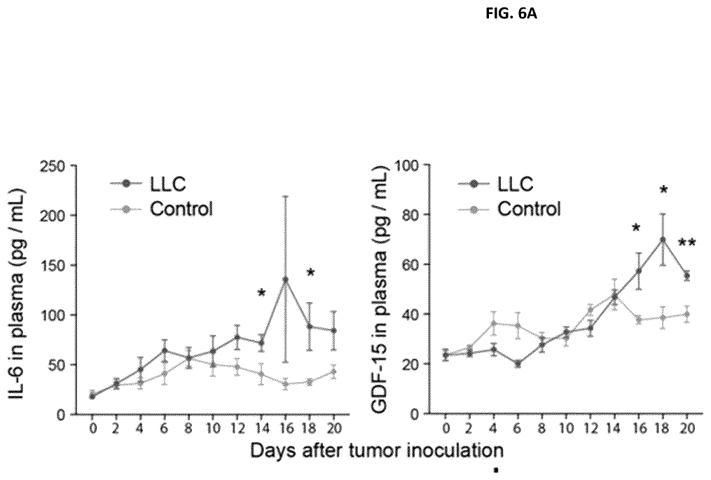

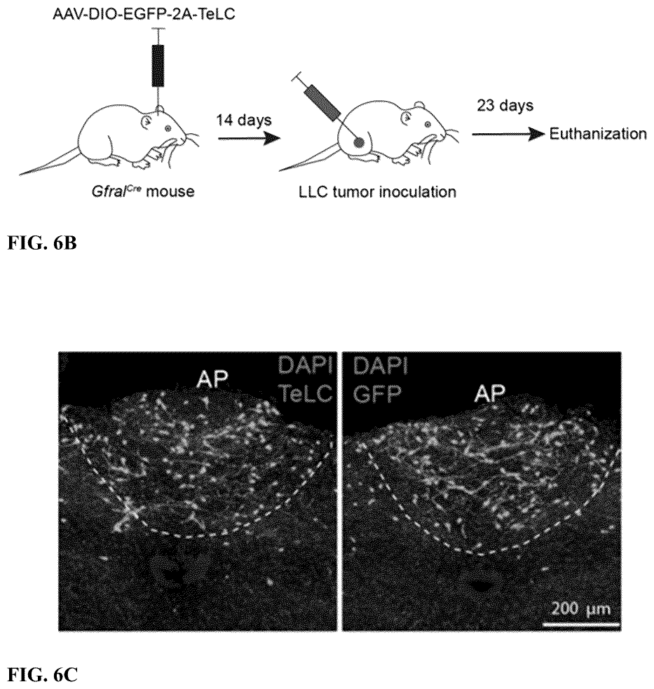

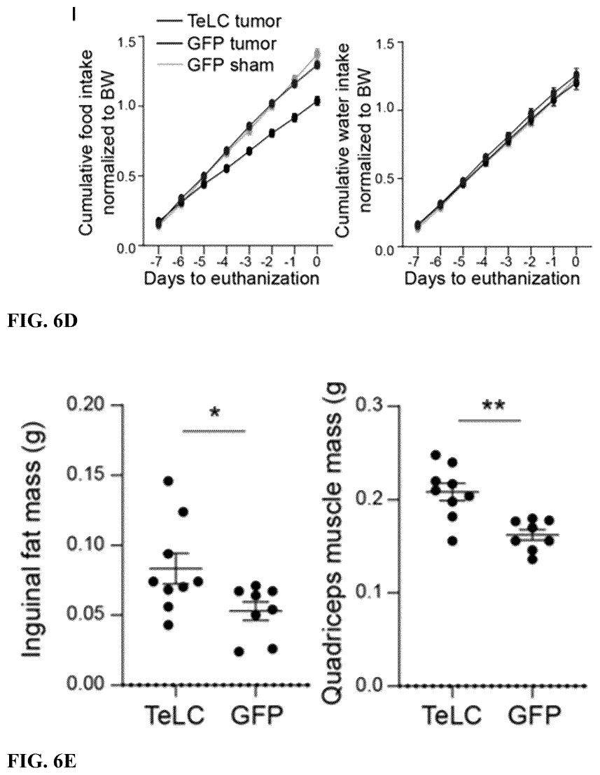

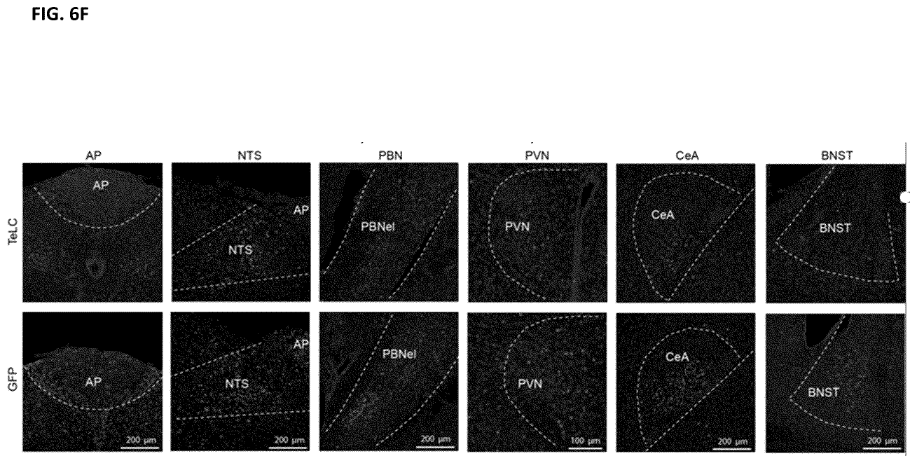

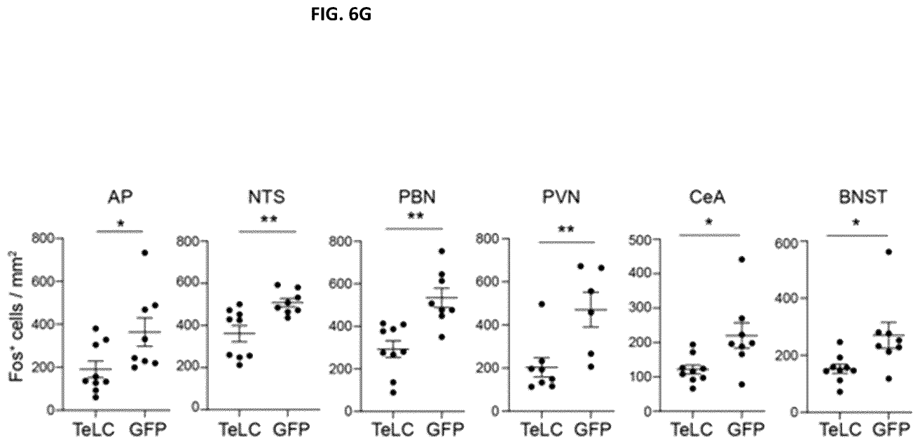

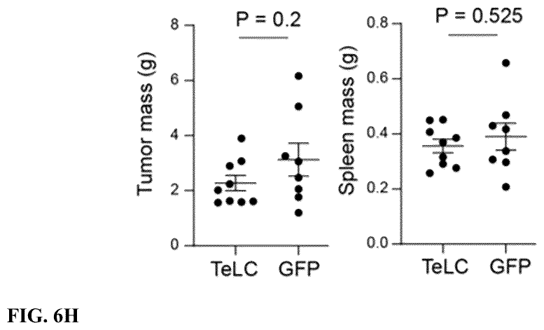

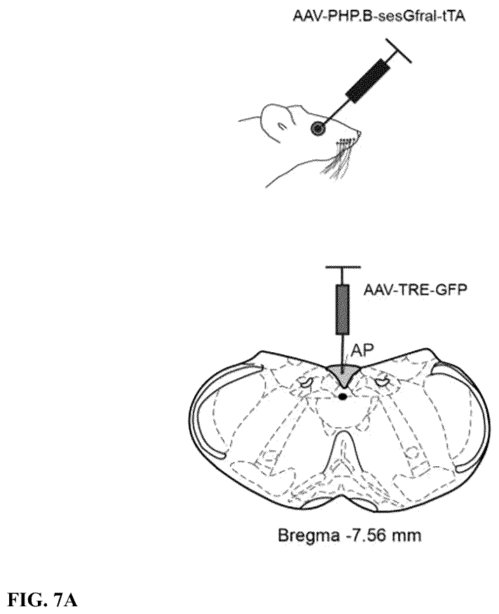

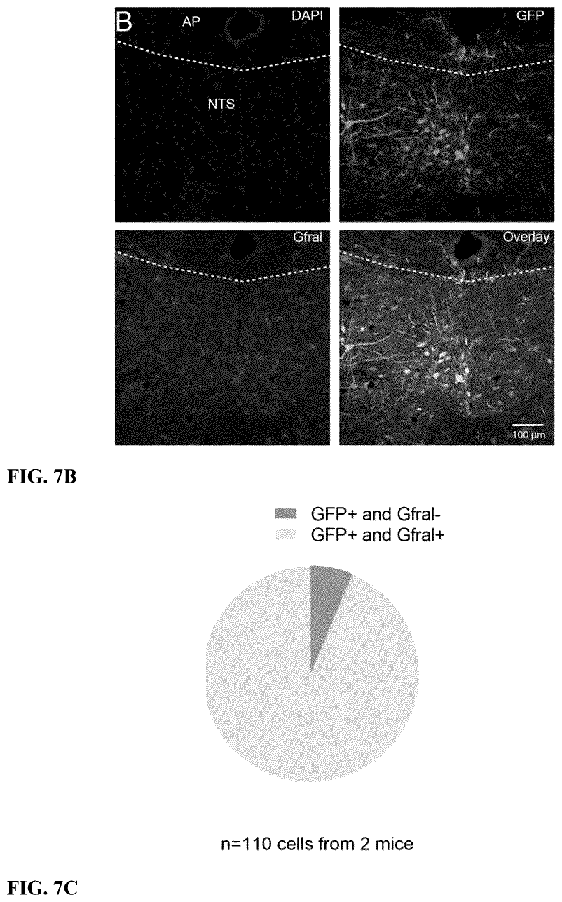

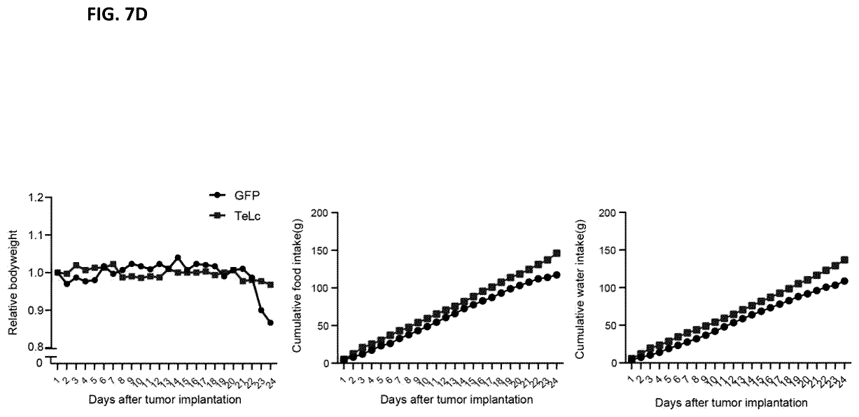

We first measured the levels of IL-6 and GDF-15 in the blood at different timepoints of cancer progression in this model. Plasma IL-6 and GDF-15 were both increased at around two weeks following tumor inoculation (FIG. 6a). To specifically inhibit the activity of Gfral+ neurons, we injected the AP of Gfral-p2a-Cre mice bilaterally with an adeno-associated virus (AAV) expressing the tetanus toxin light chain (TeLC, which blocks neurotransmitter release⁶⁵), or GFP (as a control) in a Cre-dependent manner. Two weeks later, these mice were inoculated with the LLC (FIG. 6b, 6c). Interestingly, compared with the GFP mice, the TeLC mice exhibited increased food intake at the late stage of cancer progression (FIG. 6d) and increased fat and muscle mass at the endpoint (FIG. 6e), at levels comparable to sham control mice (i.e., those without tumors). Moreover, the TeLC mice showed reduced Fos expression than the GFP mice in the AP, PBN, PVN, CeA and BNST (FIG. 6f, 6g). The two groups had similar tumor and spleen mass (FIG. 6h). These results indicate that reducing Gfral+AP neuron activity attenuates the cachectic phenotypes. In addition, other areas in the AP network are likely also involved in this process.

### Summary of Working Examples

Our study identifies AP neurons as a critical mediator of the function of IL-6 that leads to cancer-associated cachexia in mice. We show that, first, circulating IL-6 rapidly enters the AP and causes AP neuron activation within hours. Second, periphery tumors result in increased IL-6 and hyperactivity in the AP, and induce enhanced excitatory synaptic drive onto AP neurons. Third, neutralization of IL-6 in the brain of tumor-bearing mice, via i.e.v. infusion of an IL-6 antibody, prevents cachexia, counteracts the cachexia-associated hyperactivity in the AP network, and markedly prolongs lifespan. Fourth, specific suppression of//6ra in AP neurons with CRISPR/dCas9 interference has similar effects. Lastly, specific suppression of the activities of Gfral+ neurons, which partially overlap with Il6rat cells in the AP (FIG. 1h) and are involved in the effects of Il6ra suppression (FIG. 4d), also attenuates cancer-associated cachectic phenotypes and AP network hyperactivity.

The AP network showing cachexia-associated hyperactivity includes the PBN, the PVN, the BNST, and the CeA besides the AP. These structures are interconnected^{50,55-59} and have been implicated in regulating feeding behavior and metabolism^{50,58,66-73} In particular, the AP and the neighboring NTS, as well as the PBN and the PVN have previously been implicated in cancer-associated cachexia^{17,27-29}. The AP sends direct projections to the PBN and the NTS, and the NTS also directly projects to the PBN as well as the PVN^{50,59,74}. Given previous findings that both the PBN^{27,50,57,58,74-76} and the PVN^{16,77,78} are involved in feeding suppression, it is possible that the AP drives cancer-associated anorexia via the AP - - - >PBN, the AP - - - >NTS - - - >PBN, or the AP - - - >NTS - - - >PVN pathway.

A notable observation is that the AP also drives weight loss independent of anorexia during cancer progression (Data of FIG. 18), consistent with findings that cancer-associated cachexia involves active catabolic processes in addition to anorexia, and the tissue wasting can only be partially reversed by nutritional support¹⁸⁻²⁰. Interestingly, multiple nuclei in the AP network are connected with mechanisms that promote catabolic process in peripheral organs^{66,67,70,73,79} providing an anatomical basis for this function of the AP.

Recent studies indicate that Gfral is exclusively expressed by neurons in the AP and the NTS⁸⁰⁻⁸³, and systemic administration of GDF-15 activates GFRAL+ neurons in the AP and induces vomiting and anorexia^{28,50,84-87}. Furthermore, neutralization of Gfral or GDF-15 with antibodies ameliorates cancer-associated cachectic phenotypes in animals^{28,29}. Thus, GDF-15 may also influence cancer-associated cachexia, like IL-6, through the AP network. However, as GDF-15 functions as a central alert to the organism in response to a broad range of stressors⁸⁸, including infection, blockade of GDF-15/GFRAL is likely to have detrimental effects if used as a therapeutic strategy. Indeed, it has recently been shown that GDF-15 is essential for surviving bacterial and viral infections⁶⁹.

IL-6 has long been known as a key contributor to cancer-associated cachexia^{18-20,23,24,35,36} Efforts exploring IL-6 as a potential therapeutic target thus far have been focused on peripheral IL-6 or IL-6 receptors, and relied on systemic application of antibodies against these molecules 18,23,38 However, such systemic approach may not be effective in reducing IL-6 signaling in the brain. Furthermore, as IL-6 is a pleiotropic cytokine essential for immune and metabolic functions, with receptors widely distributed in the entire organism^{23,89}, systemic neutralization of IL-6 or its receptors will compromise these functions globally and likely cause severe side effects^{90,91}. Our results from multiple cancer models suggest that targeting IL-6 signaling in the brain, or more specifically in the AP, could be an effective treatment for cancer-associated cachexia.

### Methods Working Example 2

Male mice aged 2-4 months were used in all the experiments. Mice were housed under a 12-h light/dark cycle (7 a.m. to 7 p.m. light) in groups of 2-5 animals, with a room temperature (RT) of 22° C. and humidity of 50%. Food and water were available ad libitum before experiments. All experimental procedures were approved by the Institutional Animal Care and Use Committee of Cold Spring Harbor Laboratory and performed in accordance with the US National Institutes of Health guidelines. The Balbc mice (strain number: 000651) were purchased from Jackson laboratory. The Gfral-p2a-Cre mice was generated by Stephen Liberles⁵⁰.

### Immunohistochemistry

Immunohistochemistry experiments were performed following standard procedures. Briefly, mice were anesthetized with Euthasol (0.2 ml; Virbac, Fort Worth, Texas, USA) and transcardially perfused with 30 ml of PBS, followed by 30 ml of 4% paraformaldehyde (PFA) in PBS. Brains were extracted and further fixed in 4% PFA overnight followed by cryoprotection in a 30% PBS-buffered sucrose solution for 36 h at 4° C. Coronal sections (50 μm in thickness) were cut using a freezing microtome (Leica SM 2010R). Brain sections were first washed in PBS (3×5 min), incubated in PBST (0.3% Triton X-100 in PBS) for 30 min at RT and then washed with PBS (3×5 min). Next, sections were blocked in 5% normal goat serum in PBST for 30 min at RT and then incubated with the primary antibody overnight at 4° C. Sections were washed with PBS (5×15 min) and incubated with the fluorescent secondary antibody at RT for 2 h. After washing with PBS (5×15 min), sections were mounted onto slides with Fluoromount-G (eBioscience, San Diego, California, USA). Images were taken using an LSM 780 laser-scanning confocal microscope (Carl Zeiss, Oberkochen, Germany).

The primary antibodies and dilutions used in this study were: rabbit anti-Fos (1:500, Santa Cruz, sc-52), mouse anti-FLAG (1:1000, Sigma-Aldrich, F1804), rabbit anti-mCherry (1:1,000; Abcam, ab167453, GR3213077-3). The fluorophore-conjugated secondary antibodies and dilutions used were Alexa Fluor 488 goat anti-rabbit IgG (H+L; 1:500; A-11008, Invitrogen), Alexa Fluor 647 goat anti-rabbit IgG (H+L; 1:500; A-21244, Invitrogen), Alexa Fluor 594 goat anti-mouse IgG (H+L; 1:500; A-11005, Invitrogen).

### Retro-Orbital Injection of Exogenous IL-6 and its Detection in the Brain

Biotinylated human IL-6 solution (Acrobiosystems, IL-6-H8218; 2 μg/ml dissolved in saline) was injected into Balbc mice (100 μl per mouse) via retro-orbital injection. For retro-orbital injection, briefly, the animal was anaesthetized with 2% isoflurane. A 27-gauge needle on a 0.5 mL insulin syringe was used for the injection. The animal was placed on its side on a heat pad. The gauge needle was inserted at approximately a 30-45° angle to the eye, lateral to the medial canthus, through the conjunctival membrane. There is a bit of resistance that causes the eye to retreat back into the sinus until the needle pierces through the conjunctiva. The needle was positioned behind the globe of the eye in the retro-bulbar sinus. The biotinylated human IL-6 solution was injected slowly into the retro-bulbar sinus. After the injection, the needle was removed gently and the animal was returned to homecage for recovery. 3 hours after the injection, the animals were sacrificed and transcardially perfused with 30 ml of PBS, followed by 30 ml of 4% paraformaldehyde (PFA) in PBS. Brains were extracted and further fixed in 4% PFA overnight followed by cryoprotection in a 30% PBS-buffered sucrose solution for 36 h at 4° C. Coronal sections (50 μm in thickness) were cut using a freezing microtome (Leica SM 2010R). Brain sections were incubated in Streptavidin solution (1:1000, ThermoFisher, Alexa Fluor™ 647 conjugate, dissolved in 0.3% PBST) in room temperature for 2 hours. After washing with PBS (5×15 min), sections were mounted onto slides with Fluoromount-G (eBioscience). Images were taken using an LSM 780 laser-scanning confocal microscope (Carl Zeiss).

### Fluorescence In Situ Hybridization

Single molecule fluorescent in situ hybridization (smFISH) (RNAscope, ACDBio) was used to detect the expression of Glp1r, Il6ra, Fos, and Gfral mRNAs in the area postrema of Balbc mice. For tissue preparation, mice were first anesthetized under isoflurane and then decapitated. Their brain tissue was first embedded in cryomolds (Sakura Finetek, Catalog number 4566) filled with M-1 Embedding Matrix (Thermo Scientific, Catalog number 1310) then quickly fresh-frozen on dry ice. The tissue was stored at −80° C. until it was sectioned with a cryostat. Cryostat-cut sections (16-μm thick) containing the entire area postrema were collected through the rostro-caudal axis in a series of four slides, and quickly stored at −80° C. until processed. Hybridization was carried out using the RNAscope kit (ACDBio). On the day of the experiment, frozen sections were postfixed in 4% PFA in RNA-free PBS (hereafter referred to as PBS) at RT for 15 min, then washed in PBS, dehydrated using increasing concentrations of ethanol in water (50%, once; 70%, once; 100%, twice; 5 min each). Sections were then dried at RT and incubated with Protease IV for 30 min at RT. Sections were washed in PBS three times (5 min each) at RT, then hybridized. Probes against Glp1r (Catalog number 418851-C3, dilution 1:50), Il6ra (Catalog number 438931-01, dilution 1:50), Fos (Catalog number 316921-C2, dilution 1:50), and Gfral (Catalog number 417021-C2, dilution 1:50) were applied to the area postrema sections. Hybridization was carried out for 2 h at 40° C. After that, sections were washed twice in 1× Wash Buffer (Catalog number 310091; 2 min each) at RT, then incubated with the amplification reagents for three consecutive rounds (30 min, 15 min and 30 min, at 40° C.). After each amplification step, sections were washed twice in 1× Wash Buffer (2 min each) at RT. Finally, fluorescence detection was carried out for 15 min at 40° C. Sections were then washed twice in 1× Wash Buffer (2 min each), incubated with DAPI for 2 min, washed twice in 1× Wash Buffer (2 min each), then mounted with a coverslip using mounting medium. Images were acquired using an LSM780 confocal microscope equipped with 20× and 40× lenses, and visualized and processed using ImageJ and Adobe Illustrator. Cell counting and mean fluorescence intensity quantification of the images were performed with ImageJ.

Single guide RNA (sgRNA) design and lentiviral production for CRISPR/dCas9 interference sgRNAs targeting the Il6ra transcription start site (TSS) were designed using CHOPCHOP⁹². Seven Il6ra sgRNAs (sgRNA-1 to sgRNA-7) as well as a sgRNA targeting the lacZ promoter (LacZ sgRNA) were cloned into the Lenti U6-sgRNA/Ef1a-mCherry plasmid (Addgene #114199), as described previously^{93,94}. The eight sgRNA plasmids, Lenti SYN-FLAG-dCas9-KRAB-MeCP2 plasmid (Addgene #155365), and the two helper plasmids pCMV-VSV-G (Addgene #8454) and psPAX2 (Addgene #12260) were purified with the NucleoBond Xtra Midi EF kit (Takara 740420). Il6ra knockdown efficiency was assessed by transient transfection of sgRNA and dCas9-KRAB-MeCP2 into the mHypoA hypothalamic neural cell line (Cedarlane Labs, clone clu-175). 3 μg of each sgRNA plasmid and 3 μg of dCas9-KRAB-MeCP2 plasmid were co-nucleofected into 1×10⁶ mHypoA cells resuspended in a 1:1 mixture of Ingenio Electroporation reagent (Mirus Bio 50111) and OptiMEM (Gibco 31985062), using program A-033 on the Nucleofector 2b (Lonza). The cells were harvested 60 hours post-nucleofection. DAPI- and mCherry-positive cells were collected by FACS. The Il6ra mRNA was extracted and the knockdown efficiency was measured by RT-qPCR. The two most effective sgRNAs, sgRNA-4 (−23 to −41 of TSS) and sgRNA-6 (−163 to −182 of TSS), resulting in 67% and 35% knockdown of Il6ra expression in mHypoA cells, respectively, were used for in vivo experiments. FLAG-dCas9-KRAB-MeCP2, Il6ra sgRNA-4, Il6ra sgRNA-6, and lacZ sgRNA lentiviruses were produced in HEK293T cells. Lentiviral pellets were resuspended in 30 μL DPBS, aliquoted and flash-frozen on dry ice, and stored at −80° C. Physical and functional titers were obtained using the Lenti-X qRT-PCR Titration Kit (Takara 631235) and qPCR of genomic DNA following HEK293T transduction⁹⁵, respectively.

TABLE 5

| | Physical titer | Functional titer |
|---|---|---|
| | (genomic | (transducing |
| Lentiviral preparation | copies/uL) | units/uL) |
| FLAG-dCas9-KRAB-MeCP2 (prep 1) | 3.0E+11 | 1.2E+07 |
| FLAG-dCas9-KRAB-MeCP2 (prep 2) | 8.0E+10 | 1.1E+07 |
| Il6ra sgRNA-4 | 3.0E+12 | 1.4E+08 |
| Il6ra sgRNA-6 | 5.0E+11 | 3.8E+07 |
| lacZ sgRNA | 3.0E+12 | 8.8E+07 |

### Plasmids for Lentiviral Production:

lenti SYN-FLAG-dCas9-KRAB-MeCP2 was a gift from Jeremy Day (Addgene plasmid #155365; http://n2t.net/addgene: 155365; RRID:Addgene_155365) and Duke, C. G., Bach, S. V., Revanna, J. S., Sultan, F. A., Southern, N. T., Davis, M. N., Carullo, N. V. N., Bauman, A. J., Phillips, R. A., 3rd, & Day, J. J. (2020). An Improved CRISPR dCas9 Interference Tool for Neuronal Gene Suppression. Frontiers in genome editing, 2:9. https://doi.org/10.3389/fgeed.2020.00009; each of which are incorporated by reference herein. lenti U6-sgRNA/EF1a-mCherry was a gift from Jeremy Day (Addgene plasmid #114199; http://n2t.net/addgene: 114199; RRID: Addgene_114199); Savell, K. E., Bach, S. V., Zipperly, M. E., Revanna, J. S., Goska, N. A., Tuscher, J. J., Duke, C. G., Sultan, F. A., Burke, J. N., Williams, D., Ianov, L., & Day, J. J. (2019). A Neuron-Optimized CRISPR dCas9 Activation System for Robust and Specific Gene Regulation. eNeuro, 6 (1), ENEURO.0495-18.2019. https://doi.org/10.1523/ENEURO.0495-18.2019 each of which are incorporated by reference herein.

pCMV-VSV-G was a gift from Bob Weinberg (Addgene plasmid #8454; http://n2t.net/addgene: 8454; RRID: Addgene_8454); Lentivirus-delivered stable gene silencing by RNAi in primary cells. Stewart S A, Dykxhoorn D M, Palliser D, Mizuno H, Yu E Y, An D S, Sabatini D M, Chen I S, Hahn W C, Sharp P A, Weinberg R A, Novina C D. RNA 2003 Apr; 9 (4): 493-501. 10.1261/rna.2192803 PubMed 12649500; each incorporated by reference herein.

psPAX2 was a gift from Didier Trono (Addgene plasmid #12260; http://n2t.net/addgene: 12260; RRID: Addgene_12260) incorporated by reference herein.

### Adeno-Associated Viruses (AAVs)

The AAV-CMV-DIO-EGFP=2A-TeLC vector was a gift from Dr. Wei Xu at UT Southwestern. A custom virus (AAV-DJ) based on this vector was produced by WZ Biosciences Inc (Rockville, MD 20855). pAAV-hSyn-DIO-EGFP was purchased from Addgene (Watertown, MA 02472, USA). All viruses were aliquoted and stored at −80° C. until use.

### Stereotaxic Surgery

All surgery was performed under aseptic conditions and body temperature was maintained with a heating pad. Standard surgical procedures were used for stereotaxic injection. Briefly, mice were anesthetized with isoflurane (3% at the beginning and 1% for the rest of the surgical procedure), and were positioned in a stereotaxic injection frame and on top of a heating pad maintained at 35° C. A digital mouse brain atlas was linked to the injection frame to guide the identification and targeting of different brain areas (Angle Two Stereotaxic System, http://myNeuroLab.com). We used the following coordinates for injections in the area postrema: −7.65 mm from bregma, 0 mm lateral from the midline, and 4.7 mm vertical from the skull surface. For virus injection, we made a small cranial window (1-2 mm²) for each mouse, through which a glass micropipette (tip diameter, ˜5 μm) containing viral solution was lowered down to the target. For AAVs, about 0.3 μl of viral solution was injected. For lentiviruses, 0.2-0.3 μl of viral mixture (the dCas9 and the sgRNA viruses were mixed at a volume: volume ratio of 2:1) was injected. Viral solutions were delivered with pressure applications (5-20 psi, 5-20 ms at 1 Hz) controlled by a Picospritzer III (General Valve) and a pulse generator (Agilent). The speed of injection was ˜0.1 μl/10 min. We waited for at least 10 min following the injection before slowly removing the injection pipette. After injection, the incision was sealed by surgical sutures and the animal was returned to homecage for recovery.

### Colon-26 (C26) Adenocarcinoma Cells

C26 cells were cultured in complete growth medium consisting of RPMI 1640 medium with Glutamine (#11-875-093; Thermo Fisher) containing 10% of heat-inactivated Fetal Bovine Serum (FBS) (#10-438-026; Thermo Fisher) and 1× Penicillin-Streptomycin solution (#15-140-122; Thermo Fisher) under sterile conditions. 1× Trypsin-EDTA (#15400054; Thermo Fisher) was used for cell dissociation. Cells were resuspended in FBS-free RPMI and viable cells were counted using a Vi-Cell counter prior to subcutaneous injection of 2×10⁶ viable cells diluted in 100 μL RPMI into the right flank of each BALB/c mouse.

### Lewis Lung Carcinoma (LLC) Cells

LL/2 (LLC1) cells were obtained from ATCC (American Type Culture Collection; #CRL-1642) and cultured in Dulbecco's Modified Eagle's Medium (DMEM) (#30-2002; ATCC) complete growth medium, with 10% of heat-inactivated FBS (#10-438-026; Thermo Fisher) and 1× Penicillin-Streptomycin solution (#15-140-122; Thermo Fisher) under sterile conditions. 1× Trypsin-EDTA (#15400054; Thermo Fisher) was used for cell dissociation. Cells were resuspended in FBS-free DMEM and viable cells were counted using a Vi-Cell counter prior to subcutaneous injection of. 2×10⁶ viable cells were diluted in 100 μL FBS-free DMEM and were subcutaneous injected into the right flank of each C57BL/6 mouse.

### Intracerebroventricular (i.e.v.) Infusion of Anti-IL-6

On day 10 or 12 post-C26 injection, an osmotic device of 200 μL volume and a release rate of 0.5 μL/hour consisting of a cannula, connecting line, metal flow moderator and pump (#AP=2001; Alzet) was placed in a subcutaneous pocket and stereotactically implanted into the right lateral ventricle of the C26-tumour bearing BALB/c mice for a period of 14 days. Prior to use, the infusion device was assembled and equilibrated in saline overnight. The pump was filled with either the In VivoMAb rat anti-mouse IL-6 (clone MP5-20F3, #BE0046; BioXCell) or an In VivoMAb rat IgG1 isotype control (anti-HRPN, #BE0088; BioXCell). Both antibodies were diluted in PBS to achieve continuous infusion of a 5 mg/mL dose. Pump replacement surgery was performed after 14 days. The coordinate for targeting the lateral ventricle was-0.5 mm from bregma, 1.25 mm lateral from the midline, and 2.5 mm vertical from the skull surface.

### Measuring Bodyweight, Food Intake, and Water Intake

Food and water intake monitoring cages (BioDAQ Unplugged, Research Diets, Inc., New Brunswick, NJ 08901 USA) were used to measure the food intake and water intake of the animals. Mice were singly housed in these cages. Food and water were placed in an extended hopper which can be reached by the animal. The bodyweight of the animal, weight of the food and water in the hopper were measured daily at 4 μm. The cachectic mice which lost >20% of bodyweight were sacrificed and the tissues were collected for further analysis. Blood glucose concentrations were measured from whole venous blood using an automatic glucose monitor (Bayer HealthCare Ascensia Contour).

### Blood and Plasma Measurements

Blood glucose concentrations were measured from whole venous blood using an automatic glucose monitor (Bayer HealthCare Ascensia Contour). Tail vein bleeding was performed using a scalpel via tail venesection without restraint. Blood samples were collected from tail bleed using heparin-coated hematocrit capillary tubes to avoid coagulation. Samples were then centrifuged at 14,000 rpm for 5 min at 4° C. Plasma was collected in a new tube, snap frozen in liquid nitrogen and stored at −80° C. IL-6 and GDF-15 levels were measured in plasma using the mouse IL-6 Quantikine ELISA Kit (#M6000B; R&D Systems) and the Mouse/Rat GDF-15 Quantikine ELISA Kit (#MGD150; R&D) respectively.

### Brain Tissue Lysis and IL-6 Quantification

Mice were transcardially perfused with saline and the area postrema was collected, snap frozen in liquid nitrogen and stored at −80° C. until further analysis. Tissue was placed into 2-mL round-bottom homogenizer tubes pre-loaded with Stainless Steel beads (#69989; Qiagen) and filled up with lysis buffer (#AA-LYS-16 ml; RayBiotech) supplemented with Protease Inhibitor Cocktail (#AA-PI; Raybiotech) and Phosphatase Inhibitor Cocktail Set I (#AA-PHI-I; RayBiotech). Samples were homogenized in Tissue Lyser II (#85300; Qiagen) for 5 minutes and then lysates were centrifuged at 4° C. for 20 minutes at maximum speed. The supernatant was harvested and kept on ice if testing fresh or sored at −80° C. The Bicinchoninic Acid (BCA) Method was used to determine protein concentration in lysates. IL-6 levels were quantified in the lysates using a Mouse IL-6 ELISA specific for lysates (#ELM-IL6-CL-1; RayBiotech).

### In Vitro Electrophysiology

Acute slices were obtained from two-to three-month-old mice. Mice were anaesthetized with isoflurane (4%) before rapid decapitation. The brain was rapidly removed, and coronal slices (300 μm) containing the AP were cut using a HM650 Vibrating-blade Microtome (Thermo Fisher Scientific). Slices were cut in ice-cold dissection buffer (110.0 mM Choline chloride, 25.0 mM NaHCO₃, 1.25 mM NaH₂PO₄, 2.5 mM KCl, 25.0 mM glucose, 0.5 mM CaCl₂), 7.0 mM MgCl₂, 11.6 mM ascorbic acid, and 3.1 mM pyruvic acid, and bubbled with 95% O₂ and 5% CO₂) and subsequently transferred to a recovery chamber containing artificial cerebrospinal fluid (ACSF) solution (containing 118 mM NaCl, 2.5 mM KCl, 26.2 mM NaHCO₃, 1 mM NaH₂PO₄, 20 mM Glucose, 2 mM CaCl₂) and 2 mM MgCl₂, pH 7.4, and saturated with 95% O₂ and 5% CO₂) at 34° C. The slices were maintained at 34° C. for at least 40 minutes and subsequently at room temperature (20-24° C.). Recordings were made in a continuously flow of ACSF and bubbled with 95% 02/5% CO₂.

Whole-cell patch-clamp recordings were obtained with Multiclamp 700B amplifiers and pCLAMP 10 software (Molecular Devices; Sunnyvale, California, USA) and was guided using an Olympus BX51 Microscope equipped with both transmitted and epifluorescence light sources (Olympus Corporation, Shinjuku, Tokyo, Japan).

Synaptic responses were recorded at holding potentials of-70 mV (for AMPA receptor-mediated responses), and 0 mV (for GABAA receptor-mediated responses) and were low-pass filtered at 1 kHz. The internal solution for voltage-clamp experiments contained 115 mM Cesium methanesulfonate, 20 mM CsCl, 10 mM HEPES, 2.5 mM MgCl₂, 4 mM Na₂-ATP, 0.4 mM Na₃-GTP, 10 mM Na-phosphocreatine, and 0.6 mM EGTA, pH 7.2. Miniature EPSCs were recorded in the presence of tetrodotoxin (1 μM) and picrotoxin (100 μM). Spontaneous IPSCs were recorded in the presence of AP=5 (100 μM) and CNQX (5 μM). The EPSCs and IPSCs were analyzed using Mini Analysis software (Synaptosoft).

### Statistical Analysis

All statistics are indicated where used. Statistical analyses were conducted using GraphPad Prism version 6.0 (GraphPad Software, Inc., La Jolla, CA). Statistical comparisons were performed using Student's t test or ANOVA. All comparisons were two tailed. Statistic hypothesis testing was conducted at a significance level of 0.05.

### REFERENCES

1 Knupfer, H. & Preiss, R. Serum interleukin-6 levels in colorectal cancer patients--a summary of published results. Int J Colorectal Dis 25, 135-140, doi: 10.1007/s00384-009-0818-8 (2010).
2 Salgado, R. et al. Circulating interleukin-6 predicts survival in patients with metastatic breast cancer. Int J Cancer 103, 642-646, doi: 10.1002/ijc. 10833 (2003).
3 Kumari, N., Dwarakanath, B. S., Das, A. & Bhatt, A. N. Role of interleukin-6 in cancer progression and therapeutic resistance. Tumour Biol 37, 11553-11572, doi: 10.1007/s13277-016-5098-7 (2016).
4 Scott, H. R., McMillan, D. C., Crilly, A., McArdle, C. S. & Milroy, R. The relationship between weight loss and interleukin 6 in non-small-cell lung cancer. Br J Cancer 73, 1560-1562, doi: 10.1038/bjc. 1996.294 (1996).
5 Mantovani, G. et al. Cytokine activity in cancer-related anorexia/cachexia: role of megestrol acetate and medroxyprogesterone acetate. Semin Oncol 25, 45-52 (1998).
6 Ando, K. et al. Possible role for tocilizumab, an anti-interleukin-6 receptor antibody, in treating cancer cachexia. J Clin Oncol 31, e69-72, doi: 10.1200/JCO.2012.44.2020 (2013).
7 Prado, B. L. & Qian, Y. Anti-cytokines in the treatment of cancer cachexia. Ann Palliat Med 8, 67-79, doi: 10.21037/apm.2018.07.06 (2019).
8 Strassmann, G., Fong, M., Kenney, J. S. & Jacob, C. O. Evidence for the involvement of interleukin 6 in experimental cancer cachexia. J Clin Invest 89, 1681-1684, doi: 10.1172/JCI115767 (1992).
9 Baltgalvis, K. A. et al. Interleukin-6 and cachexia in ApcMin/+ mice. Am J Physiol Regul Integr Comp Physiol 294, R393-401, doi: 10.1152/ajpregu.00716.2007 (2008).
10 Baltgalvis, K. A. et al. Muscle wasting and interleukin-6-induced atrogin-I expression in the cachectic Apc (Min/+) mouse. Pflugers Arch 457, 989-1001, doi: 10.1007/s00424-008-0574-6 (2009).
11 Zaki, M. H., Nemeth, J. A. & Trikha, M. CNTO 328, a monoclonal antibody to IL-6, inhibits human tumor-induced cachexia in nude mice. Int J Cancer 111, 592-595, doi: 10.1002/ijc.20270 (2004).
12 Fujita, J. et al. Anti-interleukin-6 receptor antibody prevents muscle atrophy in colon-26 adenocarcinoma-bearing mice with modulation of lysosomal and ATP-ubiquitin-dependent proteolytic pathways. Int J Cancer 68, 637-643, doi: 10.1002/(SICI) 1097-0215 (19961127) 68: 5<637::AID-IJC14>3.0.CO; 2-Z (1996).
13 White, J. P. et al. The regulation of skeletal muscle protein turnover during the progression of cancer cachexia in the Apc (Min/+) mouse. PLOS One 6, e24650, doi: 10.1371/journal.pone.0024650 (2011).
14 Ando, K. et al. Tocilizumab, a proposed therapy for the cachexia of Interleukin6-expressing lung cancer. PLOS One 9, e102436, doi: 10.1371/journal.pone.0102436 (2014).
15 Flint, T. R. et al. Tumor-Induced IL-6 Reprograms Host Metabolism to Suppress Anti-tumor Immunity. Cell Metab 24, 672-684, doi: 10.1016/j.cmet.2016.10.010 (2016).
16 Grossberg, A. J., Scarlett, J. M. & Marks, D. L. Hypothalamic mechanisms in cachexia. Physiol Behav 100, 478-489, doi: 10.1016/j.physbeh.2010.03.011 (2010).
17 Olson, B., Diba, P., Korzun, T. & Marks, D. L. Neural Mechanisms of Cancer Cachexia. Cancers (Basel) 13, doi: 10.3390/cancers¹³¹⁶³⁹⁹⁰ (2021).
18 Biswas, A. K. & Acharyya, S. Cancer-Associated Cachexia: A Systemic Consequence of Cancer Progression. Annual Review of Cancer Biology 4, 391-411 (2020).
19 Baracos, V. E., Martin, L., Korc, M., Guttridge, D. C. & Fearon, K. C. H. Cancer-associated cachexia. Nat Rev Dis Primers 4, 17105, doi: 10.1038/nrdp.2017.105 (2018).
20 Fearon, K., Arends, J. & Baracos, V. Understanding the mechanisms and treatment options in cancer cachexia. Nat Rev Clin Oncol 10, 90-99, doi: 10.1038/nrclinonc.2012.209 (2013).
21 Tanaka, Y. et al. Experimental cancer cachexia induced by transplantable colon 26 adenocarcinoma in mice. Cancer Res 50, 2290-2295 (1990).
22 Strassmann, G. & Kambayashi, T. Inhibition of experimental cancer cachexia by anti-cytokine and anti-cytokine-receptor therapy. Cytokines Mol Ther 1, 107-113 (1995).
23 Narsale, A. A. & Carson, J. A. Role of interleukin-6 in cachexia: therapeutic implications. Curr Opin Support Palliat Care 8, 321-327, doi: 10.1097/SPC.0000000000000091 (2014).
24 Fearon, K. C., Glass, D. J. & Guttridge, D. C. Cancer cachexia: mediators, signaling, and metabolic pathways. Cell Metab 16, 153-166, doi: 10.1016/j.cmet.2012.06.011 (2012).
25 Fearon, K. C. & Carter, D. C. Cancer cachexia. Ann Surg 208, 1-5, doi: 10.1097/00000658-198807000-00001 (1988).
26 Braun, T. P. et al. Central nervous system inflammation induces muscle atrophy via activation of the hypothalamic-pituitary-adrenal axis. J Exp Med 208, 2449-2463, doi: 10.1084/jem.20111020 (2011).
27 Campos, C. A. et al. Cancer-induced anorexia and malaise are mediated by CGRP neurons in the parabrachial nucleus. Nat Neurosci 20, 934-942, doi: 10.1038/nn.4574 (2017).
28 Lerner, L. et al. MAP3K11/GDF15 axis is a critical driver of cancer cachexia. J Cachexia Sarcopenia Muscle 7, 467-482, doi: 10.1002/jcsm.12077 (2016).
29 Suriben, R. et al. Antibody-mediated inhibition of GDF15-GFRAL activity reverses cancer cachexia in mice. Nat Med 26, 1264-1270, doi: 10.1038/s41591-020-0945-x (2020).
30 Borner, T. et al. Anorexia-cachexia syndrome in hepatoma tumour-bearing rats requires the area postrema but not vagal afferents and is paralleled by increased MIC-1/GDF15. J Cachexia Sarcopenia Muscle 8, 417-427, doi: 10.1002/jcsm. 12169 (2017).
31 Borner, T., Liberini, C. G., Lutz, T. A. & Riediger, T. Brainstem GLP=1 signalling contributes to cancer anorexia-cachexia syndrome in the rat. Neuropharmacology 131, 282-290, doi: 10.1016/j.neuropharm.2017.12.024 (2018).
32 Navarro, I. B. K., Schraner, M. & Riediger, T. Brainstem prolactin-releasing peptide contributes to cancer anorexia-cachexia syndrome in rats. Neuropharmacology 180, 108289, doi: 10.1016/j.neuropharm.2020.108289 (2020).
33 Mueller, T. C., Bachmann, J., Prokopchuk, O., Friess, H. & Martignoni, M. E. Molecular pathways leading to loss of skeletal muscle mass in cancer cachexia-—can findings from animal models be translated to humans? BMC Cancer 16, 75, doi: 10.1186/s12885-016-2121-8 (2016).
34 Tisdale, M. J. Molecular pathways leading to cancer cachexia. Physiology (Bethesda) 20, 340-348, doi: 10.1152/physiol.00019,2005 (2005).
35 Tan, B. H. et al. Identification of possible genetic polymorphisms involved in cancer cachexia: a systematic review. J Genet 90, 165-177, doi: 10.1007/s12041-011-0027-4 (2011).
36 Stephens, N. A., Skipworth, R. J. & Fearon, K. C. Cachexia, survival and the acute phase response. Curr Opin Support Palliat Care 2, 267-274, doi: 10.1097/SPC.0b013e3283186be2 (2008).
37 Hirata, H. et al. Favorable responses to tocilizumab in two patients with cancer-related cachexia. J Pain Symptom Manage 46, e9-e13, doi: 10.1016/j.jpainsymman.2013.01.009 (2013).
38 Paval, D. R. et al. A systematic review examining the relationship between cytokines and cachexia in incurable cancer. J Cachexia Sarcopenia Muscle 13, 824-838, doi: 10.1002/jcsm. 12912 (2022).
39 Benrick, A. et al. Interleukin-6 gene knockout influences energy balance regulating peptides in the hypothalamic paraventricular and supraoptic nuclei. J Neuroendocrinol 21, 620-628, doi: 10.1111/j.1365-2826.2009.01879.x (2009).
40 Plata-Salaman, C. R. Anorexia induced by activators of the signal transducer gp 130. Neuroreport 7, 841-844, doi: 10.1097/00001756-199602290-00038 (1996).
41 Bossola, M., Luciani, G., Giungi, S. & Tazza, L. Anorexia, fatigue, and plasma interleukin-6 levels in chronic hemodialysis patients. Ren Fail 32, 1049-1054, doi: 10.3109/0886022X.2010.504910 (2010).
42 Rothwell, N. J. et al. Interleukin-6 is a centrally acting endogenous pyrogen in the rat. Can J Physiol Pharmacol 69, 1465-1469, doi: 10.1139/y91-219 (1991).
43 Rothwell, N. J. & Hopkins, S. J. Cytokines and the nervous system II: Actions and mechanisms of action. Trends Neurosci 18, 130-136, doi: 10.1016/0166-2236 (95) 93890-a (1995).
44 Pavlov, V. A., Chavan, S. S. & Tracey, K. J. Molecular and Functional Neuroscience in Immunity. Annu Rev Immunol 36, 783-812, doi: 10.1146/annurev-immunol-042617-053158 (2018).
45 Banks, W. A., Kastin, A. J. & Broadwell, R. D. Passage of cytokines across the blood-brain barrier. Neuroimmunomodulation 2, 241-248, doi: 10.1159/000097202 (1995).
46 Hopkins, S. J. & Rothwell, N. J. Cytokines and the nervous system. I: Expression and recognition. Trends Neurosci 18, 83-88 (1995).
47 Borison, H. L. Area postrema: chemoreceptor circumventricular organ of the medulla oblongata. Prog Neurobiol 32, 351-390, doi: 10.1016/0301-0082 (89) 90028-2 (1989).
48 Miller, A. D. & Leslie, R. A. The area postrema and vomiting. Front Neuroendocrinol 15, 301-320, doi: 10.1006/frne. 1994.1012 (1994).
49 Price, C. J., Hoyda, T. D. & Ferguson, A. V. The area postrema: a brain monitor and integrator of systemic autonomic state. Neuroscientist 14, 182-194, doi: 10.1177/1073858407311100 (2008).
50 Zhang, C. et al. Area Postrema Cell Types that Mediate Nausea-Associated Behaviors. Neuron 109, 461-472 e465, doi: 10.1016/j.neuron.2020.11.010 (2021).
51 Duquenne, M. et al. Leptin brain entry via a tanycytic LepR-EGFR shuttle controls lipid metabolism and pancreas function. Nature Metabolism 3, 1071-1090, doi: 10.1038/s42255-021-00432-5 (2021).
52 Porniece Kumar, M. et al. Insulin signalling in tanycytes gates hypothalamic insulin uptake and regulation of AgRP neuron activity. Nature Metabolism 3, 1662-1679, doi: 10.1038/s42255-021-00499-0 (2021).
53 Clayton, D. F. The genomic action potential. Neurobiol Learn Mem 74, 185-216, doi: 10.1006/nlme.2000.3967 (2000).
54 Guzowski, J. F. et al. Mapping behaviorally relevant neural circuits with immediate-early gene expression. Curr Opin Neurobiol 15, 599-606, doi: 10.1016/j.conb.2005.08.018 (2005).
55 Dong, H. W., Petrovich, G. D. & Swanson, L. W. Topography of projections from amygdala to bed nuclei of the stria terminalis. Brain Res Brain Res Rev 38, 192-246, doi: 10.1016/s0165-0173 (01) 00079-0 (2001).
56 Dong, H. W., Petrovich, G. D., Watts, A. G. & Swanson, L. W. Basic organization of projections from the oval and fusiform nuclei of the bed nuclei of the stria terminalis in adult rat brain. J Comp Neurol 436, 430-455, doi: 10.1002/cne. 1079 (2001).
57 Palmiter, R. D. The Parabrachial Nucleus: CGRP Neurons Function as a General Alarm. Trends Neurosci 41, 280-293, doi: 10.1016/j.tins.2018.03.007 (2018).
58 Roman, C. W., Derkach, V. A. & Palmiter, R. D. Genetically and functionally defined NTS to PBN brain circuits mediating anorexia. Nat Commun 7, 11905, doi: 10.1038/ncomms11905 (2016).
59 Song, C. K., Schwartz, G. J. & Bartness, T. J. Anterograde transneuronal viral tract tracing reveals central sensory circuits from white adipose tissue. Am J Physiol Regul Integr Comp Physiol 296, R501-511, doi: 10.1152/ajpregu.90786.2008 (2009).
60 Duke, C. G. et al. An Improved CRISPR/dCas9 Interference Tool for Neuronal Gene Suppression. Front Genome Ed 2, 9, doi: 10.3389/fgeed.2020.00009 (2020).
61 Yeo, N. C. et al. An enhanced CRISPR repressor for targeted mammalian gene regulation. Nat Methods 15, 611-616, doi: 10.1038/s41592-018-0048-5 (2018).
62 Michaelis, K. A. et al. Establishment and characterization of a novel murine model of pancreatic cancer cachexia. 8, 824-838, doi: https://doi.org/10.1002/jcsm. 12225 (2017).
63 Olson, B. et al. Lipocalin 2 mediates appetite suppression during pancreatic cancer cachexia. Nature Communications 12, 2057, doi: 10.1038/s41467-021-22361-3 (2021).
64 Bonetto, A., Rupert, J. E., Barreto, R. & Zimmers, T. A. The Colon-26 Carcinoma Tumor-bearing Mouse as a Model for the Study of Cancer Cachexia. J Vis Exp, doi: 10.3791/54893 (2016).
65 Xu, W. et al. Distinct neuronal coding schemes in memory revealed by selective erasure of fast synchronous synaptic transmission. Neuron 73, 990-1001, doi: 10.1016/j.neuron.2011.12.036 (2012).
66 Cardoso, F. et al. Neuro-mesenchymal units control ILC2 and obesity via a brain-adipose circuit. Nature 597, 410-414, doi: 10.1038/s41586-021-03830-7 (2021).
67 Deuchars, S. A. & Lall, V. K. Sympathetic preganglionic neurons: properties and inputs. Compr Physiol 5, 829-869, doi: 10.1002/cphy.c140020 (2015).
68 Koren, T. et al. Insular cortex neurons encode and retrieve specific immune responses. Cell 184, 5902-5915 e5917, doi: 10.1016/j.cell.2021.10.013 (2021).
69 Luan, H. H. et al. GDF15 Is an Inflammation-Induced Central Mediator of Tissue Tolerance. Cell 178, 1231-1244 e1211, doi: 10.1016/j.cell.2019.07.033 (2019).
70 Nandi, J. et al. Central mechanisms involved with catabolismurr Opin Clin Nutr Metab Care 5, 407-418, doi: 10.1097/00075197-200207000-00010 (2002).
71 Sternson, S. M. & Eiselt, A. K. Three Pillars for the Neural Control of Appetite. Annu Rev Physiol 79, 401-423, doi: 10.1146/annurev-physiol-021115-104948 (2017).
72 Zhang, X. et al. Brain control of humoral immune responses amenable to behavioural modulation. Nature 581, 204-208, doi: 10.1038/s41586-020-2235-7 (2020).
73 Papazoglou, I. et al. A distinct hypothalamus-to-beta cell circuit modulates insulin secretion. Cell Metab 34, 285-298 e287, doi: 10.1016/j.cmet.2021.12.020 (2022).
74 Xie, Z. et al. The gut-to-brain axis for toxin-induced defensive responses. Cell 185, 4298-4316 e4221, doi: 10.1016/j.cell.2022.10.001 (2022).
75 Chen, J. Y., Campos, C. A., Jarvie, B. C. & Palmiter, R. D. Parabrachial CGRP Neurons Establish and Sustain Aversive Taste Memories. Neuron 100, 891-899 e895, doi: 10.1016/j.neuron.2018.09.032 (2018).
76 Sabatini, P. V. et al. GFRAL-expressing neurons suppress food intake via aversive pathways. Proc Natl Acad Sci USA 118, doi: 10.1073/pnas.2021357118 (2021).
77 Yoo, E. S. et al. Galpha (i/o)—coupled Htr²c in the paraventricular nucleus of the hypothalamus antagonizes the anorectic effect of serotonin agents. Cell Rep 37, 109997, doi: 10.1016/j.celrep.2021.109997 (2021).
78 Pei, H., Sutton, A. K., Burnett, K. H., Fuller, P. M. & Olson, D. P. AVP neurons in the paraventricular nucleus of the hypothalamus regulate feeding. Mol Metab 3, 209-215, doi: 10.1016/j.molmet.2013.12.006 (2014).
79 Morrison, S. F. RVLM and raphe differentially regulate sympathetic outflows to splanchnic and brown adipose tissue. Am J Physiol 276, R962-973, doi: 10.1152/ajpregu. 1999,276.4.R962 (1999).
80 Emmerson, P. J. et al. The metabolic effects of GDF15 are mediated by the orphan receptor GFRAL. Nat Med 23, 1215-1219, doi: 10.1038/nm.4393 (2017).
81 Hsu, J. Y. et al. Non-homeostatic body weight regulation through a brainstem-restricted receptor for GDF15. Nature 550, 255-259, doi: 10.1038/nature24042 (2017).
82 Mullican, S. E. et al. GFRAL is the receptor for GDF15 and the ligand promotes weight loss in mice and nonhuman primates. Nat Med 23, 1150-1157, doi: 10.1038/nm.4392 (2017).
83 Yang, L. et al. GFRAL is the receptor for GDF15 and is required for the anti-obesity effects of the ligand. Nat Med 23, 1158-1166, doi: 10.1038/nm.4394 (2017).
84 Johnen, H. et al. Tumor-induced anorexia and weight loss are mediated by the TGF-beta superfamily cytokine MIC-1. Nat Med 13, 1333-1340, doi: 10.1038/nm1677 (2007).
85 Breen, D. M. et al. GDF-15 Neutralization Alleviates Platinum-Based Chemotherapy-Induced Emesis, Anorexia, and Weight Loss in Mice and Nonhuman Primates. Cell Metab 32, 938-950 e936, doi: 10.1016/j.cmet.2020.10.023 (2020).
86 Altena, R. et al. Growth differentiation factor 15 (GDF-15) plasma levels increase during bleomycin- and cisplatin-based treatment of testicular cancer patients and relate to endothelial damage. PLOS One 10, e0115372, doi: 10.1371/journal.pone.0115372 (2015).
87 Tsai, V. W. W., Husaini, Y., Sainsbury, A., Brown, D. A. & Breit, S. N. The MIC-1/GDF15-GFRAL Pathway in Energy Homeostasis: Implications for Obesity, Cachexia, and Other Associated Diseases. Cell Metab 28, 353-368, doi: 10.1016/j.cmet.2018.07.018 (2018).
88 Lockhart, S. M., Saudek, V. & O'Rahilly, S. GDF15: A Hormone Conveying Somatic Distress to the Brain. Endocr Rev 41, doi: 10.1210/endrev/bnaa007 (2020).
89 Heinrich, P. C. et al. Principles of interleukin (IL)-6-type cytokine signalling and its regulation. Biochem J 374, 1-20, doi: 10.1042/BJ20030407 (2003).
90 Berti, A., Boccalatte, F., Sabbadini, M. G. & Dagna, L. Assessment of tocilizumab in the treatment of cancer cachexia. J Clin Oncol 31, 2970, doi: 10.1200/JCO.2012.48.4147 (2013).
91 Kopf, M. et al. Impaired immune and acute-phase responses in interleukin-6-deficient mice. Nature 368, 339-342, doi: 10.1038/368339a0 (1994).
92 Labun, K. et al. CHOPCHOP v3: expanding the CRISPR web toolbox beyond genome editing. Nucleic Acids Res 47, W171-W174, doi: 10.1093/nar/gkz365 (2019).
93 Savell, K. E. et al. A Neuron-Optimized CRISPR/dCas9 Activation System for Robust and Specific Gene Regulation. eNeuro 6, doi: 10.1523/ENEURO.0495-18.2019 (2019).
94 Savell, K. E., Sultan, F. A. & Day, J. J. A Novel Dual Lentiviral CRISPR-based Transcriptional Activation System for Gene Expression Regulation in Neurons. Bio Protoc 9, e3348, doi: 10.21769/BioProtoc.3348 (2019).
95 Gordon, M. G. et al. lentiMPRA and MPRAflow for high-throughput functional characterization of gene regulatory elements. Nat Protoc 15, 2387-2412, doi: 10.1038/s41596-020-0333-5 (2020).
96 Kern, L., Mittenbühler, M. J., Vesting, A. J., Ostermann, A. L., Wunderlich, C. M., & Wunderlich, F. T. (2018). Obesity-Induced TNFα and IL-6 Signaling: The Missing Link between Obesity and Inflammation-Driven Liver and Colorectal Cancers. Cancers, 11 (1), 24. https://doi.org/10.3390/cancers¹¹⁰¹⁰⁰²⁴

1. A modular RNA molecule comprising:
(i) a 5′ region comprising a sensor domain comprising a stretch of consecutive nucleotides that is complementary to a stretch of consecutive nucleotides of a selected cellular RNA of neuron or neuronal cell of the area postrema of the mammalian central nervous systems that encodes the Gfral gene, wherein the sensor domain comprises at least one stop codon editable by ADAR; and
(ii) a 3′ region comprising a domain encoding one or more effector proteins selected from one or more of the group consisting of a label, a transcriptional activator, and a transcriptional repressor, wherein the protein coding domain is downstream of and in-frame with the sensor domain,
wherein, upon introduction of the modular RNA into a cell comprising an Adar enzyme, the stretch of consecutive nucleotides of the sensor domain and the corresponding nucleotide stretch of the cellular RNA form an RNA duplex comprising the stop codon, wherein the stop codon comprised in the RNA duplex is edited by ADAR in the cell, thereby to permit translation of the protein.

2. The modular RNA molecule of claim 1, in which the effector protein comprises a transcription activator that increases the activity of Gfral-expressing (Gfral+) AP neurons.

3. The modular RNA molecule according to claim 2 in which the transcriptional activator is selected from the group consisting of: IL6a, sodium channel, mutant AMPA receptor, GluA4, and combinations thereof.

4. The modular RNA molecule according to claim 3 in which the transcriptional activator comprises a sodium channel.

5. The RNA molecule according to claim 4 in which the sodium channel comprises mNaChBac.

6. The RNA molecule according to claim 3 in which the transcriptional activator comprises a mutant AMPA receptor.

7. The RNA molecule according to claim 6 in which the mutant AMPA receptor comprises GluA2-LA83Y-R845A.

8. The modular RNA molecule according to claim 1 in which the effector protein comprises a transcriptional repressor that decreases the activity of Gfral-expressing (Gfral+) AP neurons.

9. The modular RNA molecule according to claim 8 in which the transcriptional repressor is selected from the group consisting of: IL6aR, Tetanus Toxin Light Chain (TeLC), a dominant negative Ras, a dominant negative STAT3, GluA4 C-tail, and combinations thereof.

10. The modular RNA molecule according to claim 1 in which the molecule further encodes a self-cleaving 2A peptide positioned between the sensor domain and the 3′ protein coding domain.

11. A composition comprising:
i) a first nucleic acid comprising a modular RNA molecule comprising:
(a) a sensor domain comprising a stretch of consecutive nucleotides that is complementary to a corresponding stretch of consecutive nucleotides of a selected cellular RNA of neuron or neuronal cell of the area postrema of the mammalian central nervous systems that encodes the Gfral gene, wherein the sensor domain comprises at least one stop codon editable by ADAR; and
(b) a first protein-coding domain encoding an effector protein selected from the group consisting of a label, a transcriptional activator, and a transcriptional repressor, wherein the first protein-coding region is downstream of and in-frame with the sensor domain, and
ii) a second nucleic acid comprising a second protein coding domain,
wherein, upon introduction of the nucleic acid into a cell comprising an Adar enzyme, the stretch of consecutive nucleotides of the sensor domain and the corresponding nucleotide stretch of the cellular RNA form an RNA duplex comprising the stop codon, wherein the stop codon comprised in the RNA duplex is edited by ADAR in the cell, thereby to permit translation of the protein.

12. A nucleic acid delivery vehicle comprising: (i) the modular RNA molecule of claim 1 and/or (ii) DNA encoding the modular RNA molecule of claim 1.

13. The modular RNA molecule of claim 1 in which the modular RNA molecule is encoded by a DNA vector.

14. The composition of claim 11, in which said first and second nucleic acid are encoded by one or more DNA vectors.

15. A pharmaceutical composition comprising the modular RNA molecule of claim 1 or delivery vehicle thereof, or cell thereof, and a pharmaceutically acceptable carrier, excipient and/or diluent.

16. A cell comprising: (i) the modular RNA molecule of claim 1, or (ii) a composition comprising said modular RNA molecule thereof or (iii) a delivery vehicle thereof.

17. A kit comprising the modular RNA molecule of claim 1, a composition thereof, a delivery vehicle thereof, or a pharmaceutical composition thereof and packaging and/or instructions therefore.

18. A method for treating a disease or disorder in a mammal, the method comprising administering to a subject in need thereof a therapeutically effective amount of a modular RNA molecule of claim 1, a composition thereof, a delivery vehicle thereof, a pharmaceutical composition thereof or a cell thereof to permit translation of the 3′ encoded protein or the effector protein in selected cells of the subject, thereby to produce the protein in the cells, wherein production of the protein in the cells provides for treatment of the disease or disorder in the mammal.

19. The method according to claim 18 in which the disease or disorder is selected from the group consisting of obesity and cachexia.

20. A nucleic acid delivery vehicle comprising the nucleic acid composition of claim 11, and/or DNA encoding the composition of claim 11.

21. A method to suppress IL-6 mediated neural activity in the area postrema (AP) of a mammal said method comprising administering to said mammal a therapeutically effective amount of an agent, said agent comprising
(i) a modular RNA molecule according to claim 1 or a nucleic acid composition thereof or a delivery vehicle thereof, wherein said stretch of consecutive nucleotides of said sensor domain is complementary to a stretch of consecutive nucleotides of a selected cellular RNA of an AP neuron cell encoded by a gene encoding Gfral , and wherein said 3′ region comprises a transcription activator and a first effector protein, and
(ii) a second nucleic acid comprises a coding region for a second effector protein under the control of an inducible promoter induced by said transcriptional activator, wherein said second effector protein is a suppressor of said selected cellular RNA,
(iii) wherein said decrease in the amount of said selected cellular Gfral RNA in said mammal reduces IL-6 mediated neural activity in the area postrema (AP) of said mammal.

22. The method of claim 21, wherein said mammal is afflicted with cancer-associated cachexia, and wherein said reduction IL-6 mediated neural activity in the area postrema (AP) of said mammal decreases the cancer-associated cachexia in said mammal.

23. The method of claim 21, wherein the suppressor of said selected cellular RNA is selected from the group consisting of: IL6ra shRNA, tetanus toxin light chain (TeLC); a dominant Ras, a dominant negative STAT3 and GluA4.

24. The method of claim 21, wherein said stretch of consecutive nucleotides of said sensor domain is complementary to a stretch of consecutive nucleotides of a selected cellular RNA of an AP neuron cell.

25. A method to increase IL-6 mediated neural activity in the area postrema (AP) of a mammal, said method comprising administering to said mammal a therapeutically effective amount of an agent, said agent comprising:
(i) a modular RNA molecule according to claim 1 or a nucleic acid composition thereof, or a delivery vehicle thereof, wherein said stretch of consecutive nucleotides of said sensor domain is complementary to a stretch of consecutive nucleotides of a selected cellular RNA of an AP neuron cell encoded by a gene encoding Gfral , and wherein said 3′ region comprises a transcription activator and a first effector protein, and
(ii) a second nucleic acid comprises a coding region for a second effector protein under the control of an inducible promoter induced by said transcriptional activator, wherein said second effector protein is an activator of said selected cellular RNA,
(iii) wherein a decrease in the amount of said selected cellular RNA increases IL-6 mediated neural activity in the area postrema (AP) of said mammal afflicted with obesity, thereby decreasing obesity in said mammal afflicted with obesity.

26. The method of claim 25, wherein said mammal is afflicted with obesity and wherein said increase in IL-6 mediated neural activity in the area postrema (AP) of said mammal decreases the obesity in said mammal.

27. The method of claim 25, wherein the activator of said selected cellular RNA is selected from the group consisting of: IL6rα, a sodium channel, the sodium channel mNaChBac, a mutant AMPA receptor, and GluA4.


SEQUENCE LISTING (28 sequences)

SEQ ID NO: 1 - DNA, 63 bp, unidentified
  source 1..63: mol_type = genomic DNA; note = porcine teschovirus-1
ggaagcggagctactaacttcagcctgctgaagcaggctggagacgtggagaaccctggacct

SEQ ID NO: 2 - protein, 22 aa, unidentified
  source 1..22: note = porcine teschovirus-1
GSGATNFSLLKQAGDVEENPGP

SEQ ID NO: 3 - DNA, 901 bp, synthetic construct
  source 1..901: mol_type = other DNA
catcattttggcaaagaattggatccgccaccatgggagaactactctatgttgttgtgtgcatggcagttacctgtggaattcttttcttggtgatgctcaagttaaggatacaaagtgaaaaaagagatccctcatccatcgaaatagctggaggtgtcatcattcagtgagctgcagatcacttaccaaccacatgtctgtgtgactaaccaatggaaaattacatttgccaataacgcaatttaagatggatttgacaatatttagtcattatatgtaacagtgactggtacagtaatataccacaatgatcacagatctgtttttgtttttgtttttaatgtttgagtaaatacttgttgtggtgtcataactagttgataacattttctttaaagacaacaggtgtcatgtaaaatgtgacaaatttgctggaagactatcaatccacatatcaacttctatcttatggaactaatcataattagtgtgtgcagttttctgaacaaggttatagttttccattaagttggtaaaattaaaatgctaagtagaatattgagtatacttgttatttatatattcttacttagtgtccaatcattaaacaaattggtaacattgaacatatttagttagatgactgcttatgaaaataagaactgacatcttacaaattttataatttaaatagtattgaattttactttttatttggtatgttaagattcataatatataaagcagctacattggttgagaaaagtcaatggttactccagtaatgatatactttgtgaatttatttatttttgctaattaatgatcctgaatgtaatcatgatgaaataaaaaagacatacttaaaaagcttatcgataatcaacctctggattac

SEQ ID NO: 4 - DNA, 303 bp, synthetic construct
  source 1..303: mol_type = other DNA
gacgtggaggagaaccctggacctgaattctgcaaagctgatatgagtaccagtactggtatttataactctagcatgaagacttccttatattgcaatttacctcttcattctaatttattgctctttgattttgtgtagttgcattcactctcctgccaggtcaatgtgactaaattaattccaatacaaacagaaaaggtcttaagtaggattgaatggtacttttgttttgtgctttgttgctctgtcaggctggattttgctcaccaaggcgcgccagagggcagaggaagtcttcta

SEQ ID NO: 5 - DNA, 316 bp, synthetic construct
  source 1..316: mol_type = other DNA
ggaggagaaccctggacctgaattctttaagtatgtcttttttatttcatcatgcttacattcaggatcattaattagcaaaaataaagaaattcacaaagtatatcattactagagtaaccattgccttttctcaaccaatgtagctgctttatatattatgaatcttaacataccaaataaaaagaaaattcaatactatttaaattataaaatttgtaagatgtcagttcttattttcataagcagtcatctaactaaatatgttcaatgttaccaatttgttggcgcgccagagggcagaggaagtcttcta

SEQ ID NO: 6 - DNA, 301 bp, synthetic construct
  source 1..301: mol_type = other DNA
ggaggagaaccctggacctgaattcaaagtaaaattcaatactattgaaattataaaatttgtaagatgtcagttcttattttcatacgcagtcatcgaactaaatatgttcaatgttaccaatttgtttaatgattagacactaagtaagaatatatacataacaagtatactcaatattctacttagcattttaattttaccaacttactggaaaactataaccttgttcagaaaactgcacacactaattatcattagttccataagaggcgcgccagagggcagaggaagtcttcta

SEQ ID NO: 7 - DNA, 295 bp, synthetic construct
  source 1..295: mol_type = other DNA
ggaggagaaccctggacctgaattcctaaatatgttcaatgttaccaatttgtttaatgattggacactaagtaagaatatacaaataacaagtatactcaatattctacttagcattttaattttaccaactgaatagaaaactataaccttgttcagaaaactgcacacactaattatgattagttccataagatacaagttgatatgtggattgatagtcttccagcaaatttgtcacattttacatgacacctgttgtcttggcgcgccagagggcagaggaagtcttcta

SEQ ID NO: 8 - DNA, 298 bp, synthetic construct
  source 1..298: mol_type = other DNA
ggaggagaaccctggacctgaattcatttgtcacattttacatgacacctgttgtctttacagaaaatgttatcaactagttatgacaccacaacaagtatttactcaaacattaaaaacaaaaacaaaaacagatctgcgatcattgtagtatattactgtaccagtcactgttacatataatgactacatattgtcaaatccatcttacattgcgttattggcaaatgtaattttccattggttagtcacacagacatgtggttggggcgcgccagagggcagaggaagtcttcta

SEQ ID NO: 9 - DNA, 295 bp, synthetic construct
  source 1..295: mol_type = other DNA
ggaggagaaccctggacctgaattccaaacattaaaaacaaaaacaaaaacagatctgtgctcattgtggtatattactgtaccagtcactgttacatataatgacgaaatattgtcaaatccatcttacattgcgttattggcaaatgtaattttccattagttagtcacacagacatgtggttggtacgtgatctgcagctcactgaatgatgacacctccagctatttcgatggatgtgggatctcttttttcactttgtatggcgcgccagagggcagaggaagtcttcta

SEQ ID NO: 10 - DNA, 286 bp, synthetic construct
  source 1..286: mol_type = other DNA
ggaggagaaccctggacctgaattcaaacaaaaacaaaaacagatctgtgatcattgtggtatattactgtaccagtcactgttacatataatgactaaatattgtcaaatccatcttaaattgcgttattagcaaatgtacattttccattagttagtcacacagacatgtggttggtacgtgatctgcagctcactgaatgatgacacctccagctatttcgatggatgtgggatctcttttttcactttgtatggcgcgccagagggcagaggaagtcttcta

SEQ ID NO: 11 - DNA, 295 bp, synthetic construct
  source 1..295: mol_type = other DNA
ggaggagaaccctggacctgaattctcaaacattaaaaacaaaaacaaaaacagatctgtgatcattgtggtatattactgtaccagtcactgttacatatactgcctaaatattgtcaaatccatcttaaattgcgttattggcaaatgtaattttccattggttagtcacacagacatgtagttggtaagtgatctgcagctcactgaagatgacacctccagctatttcgatggatgtgggatctcttttttcactttgtatggcgcgccagagggcagaggaagtcttcta

SEQ ID NO: 12 - DNA, 337 bp, synthetic construct
  source 1..337: mol_type = other DNA
ggagaaccctggacctgaattcggcaagtggggggtggaactatattcagtgcacatggcttgctgtgaagagtttctttggattgctgacagggtatatcagattgagcacagtcacaaaaagccaacatctgggcagtgtgaaaaggcatattttgatagaagaaccgtatagctgcttggcagtgtttcacatcacacagatttccatttgctgagcgtgcttgaaggtaaagggccaactgtgcattacaaaccacatcccctacacacgcctctgtcacctccaaacaagactgcatctgttggcgcgccagagggcagaggaagtcttcta

SEQ ID NO: 13 - DNA, 367 bp, synthetic construct
  source 1..367: mol_type = other DNA
ggagaaccctggacctgaattccaactgtgcattacaaaccacatcccctacacacgcctctgtcacctccaaacaagactgcatctgtttgaatccatgatagaaaggagtagtttagattccatttatctttattgtccttttccatgttatctgtcttattagtgcacttttttccaaaaagtttgtttactgtacagtggaggtcatccatacaaagacactcttttaaattggaaatttttttccaccaaagcctggatactcaggttacaatgtagtgaattatttatcttgcaggagtcacctggagtaaggcaggtgtcttccattgatggcgcgccagagggcagaggaagtcttcta

SEQ ID NO: 14 - DNA, 367 bp, synthetic construct
  source 1..367: mol_type = other DNA
ggagaaccctggacctgaattccatgctaatgcaggtctcatcttcatggcacttcccagttatgtggggccagcattctgtctggaatgttcggtagtgtgtcctgcagaattcatcatttcggcaagtgtgaattacactgaggcaagtggggggtggaactatattcagtgcacatagcttgctgtgaagagtttctttggattgctgacagggtatatcagattgagcacagtcacaaaaagccaacatctgggcagtgttaaaaggcatattttgatagaagaaccgtatggctgcttggcagtgtttcacatcacacagatttccatttgcggcgcgccagagggcagaggaagtcttcta

SEQ ID NO: 15 - DNA, 367 bp, synthetic construct
  source 1..367: mol_type = other DNA
ggagaaccctggacctgaattccatgctaatgcaggtctcatcttcatggcacttcccagttatgtggggccagcattctgtctggaatgttcggtagtgtgtcctgcagaattcatcatttcggcaagtgtgaattacactgaggcaagtggggggtagaactatattcagtgcacatagcttgctgtgaagagtttctttggattgctgacagggtatatcagattgagcacagtcacaaaaagccaacatctgggcagtgttaaaaggcatattttgatagaagaaccgtatggctgcttggcagtgtttcacatcacacagatttccatttgcggcgcgccagagggcagaggaagtcttcta

SEQ ID NO: 16 - DNA, 401 bp, synthetic construct
  source 1..401: mol_type = other DNA
ggagaaccctggacctgaattcaaaggagtagtagttagattccatttatctttattgtccttttccatgttatctgtcttattggtgcacttttttccaaaaagtttgtttactgtacagtggaggtcatccatacaaagacactctttaaattggaaatttttttccaccaaagcctagatactcaggttacaatgtagtgaattatttatcttgcaggagtcacctggagtaaggcaggtgtcttccattgatctccatgactgctcacagccatttgcatcaatcaagcatttctgtattaaatgtgcacaatcatttgtttgggaagaggattcattttctgagcttaacgtaacagccaggaaaaggcgcgccagagggcagaggaagtcttcta

SEQ ID NO: 17 - DNA, 343 bp, synthetic construct
  source 1..343: mol_type = other DNA
ggagaaccctggacctgaattcaaaggagtagtagttagattccatttatctttattgtccttttccatgttatctgtcttattggtgcacttttttccaaaaagtttgtttactgtacagtggaggtcatccatacaaagacactctttaaattagaaatttttttccaccaaagcctagatactcaggttacaatgtagtgaattatttatcttgcaggagtcacctggagtaaggcaggtgtcttccattgatctccatgactgctcacagccatttgcatcaatcaagcatttctgtattaaatgtgcaggcgcgccagagggcagaggaagtcttcta

SEQ ID NO: 18 - DNA, 499 bp, synthetic construct
  source 1..499: mol_type = other DNA
ggagaaccctggacctgaattcgatgagggatctcttttttcactttgtatccttaacttgagcatcaccaagaaaagaattccacagaaatgctggaaaatcatgcacacgtgttcttcagcctgtgtaacgcccctgcaagcacagggtaaacaggtctcatcttcatggcacttcccagttatgtggggccagcattctgtctggaatgttcggaaaagtcacaaaaagccaacatctgggcagtgtgaaaaggcatattttgatagaagaaccgtatagctgcttggcagtgtttcacatcacacagatttccatttgctgagcgtgcttgaaggtaaattatctgtcttattggtgcacttttttccaaaaagtttgtttactgtacagtggaggtcaaaatgtgcacaatcatttgtttgggaagaggattcattttctgagcttaacgtaacagccaggaaaggcgcgccagagggcagaggaagtcttcta

SEQ ID NO: 19 - DNA, 466 bp, synthetic construct
  source 1..466: mol_type = other DNA
ggagaaccctggacctgaattcgatgagggatctcttttttcactttgtatccttaacttgagcatcaccaagaaaagaattccacagaaatgctggaaaatcatgcacacgtgttcttcagcctgtgtaacgcccctgcaagcacagggtaaaagcattctgtctggaatgttcggtagtgtgtcctgcataattcatcatttcggcaagtgtaaatctgtcttattggtgcacttttttccaaaaagtttgtttactgtacagtggaggtcatccatacaaagacactctttaaattagaaatttttttccaccaaagcctggatactcaggttacaatgtagtgaattatttatcttgcaggagtcaaaatgtgcacaatcatttgtttgggaagaggattcattttctgagcttaacgtaacagccaggaaaggcgcgccagagggcagaggaagtcttcta

SEQ ID NO: 20 - DNA, 547 bp, synthetic construct
  source 1..547: mol_type = other DNA
ggagaaccctggacctgaattcgatgagggatctcttttttcactttgtatccttaacttgagcatcaccaagaaaagaattccacagaaatgctggaaaatcatgcacacgtgttcttcagcctgtgtaacgcccctgcaagcacagggtaaaataattcatcatttcggcaagtgtgaattacacggaggcaagtggggggtggaactatattcagtgcacatggcttgctgtgaagagtttctttagattgctgacagggtatatcagattgagcacagtcacaaaaagccaacatctgggcagtgtgaaaaggcatattttgatagaagaaccgtatggctgcttggcagtgtttcacatcacacagatttccatttgctgagcaaattatctgtcttattggtgcacttttttccaaaaagtttgtttactgtacagtggaggtcaaaatgtgcacaatcatttgtttgggaagaggattcattttctgagcttaacgtaacagccaggaaaggcgcgccagagggcagaggaagtcttcta

SEQ ID NO: 21 - DNA, 475 bp, synthetic construct
  source 1..475: mol_type = other DNA
ggagaaccctggacctgaattcgatgagggatctcttttttcactttgtatccttaacttgagcatcaccaagaaaagaattccacagaaatgctggaaaatcatgcacacgtgttcttcagcctgtgtaacgcccctgcaagcacagggtaaaggggtggaactatattcagtgcacatggcttgctgtgaagagtttctttagattgctgacagggtatatcagattgagcacagtcacaaaaagccaacaaaaggtaaagggccaactgtgcattacaaaccacatcccctacacacgcctctgtcacctccaaaattatctgtcttattggtgcacttttttccaaaaagtttgtttactgtacagtggaggtcaaaatgtgcacaatcatttgtttgggaagaggattcattttctgagcttaacgtaacagccaggaaaggcgcgccagagggcagaggaagtcttcta

SEQ ID NO: 22 - DNA, 616 bp, synthetic construct
  source 1..616: mol_type = other DNA
ggagaaccctggacctgaattcgatgagggatctcttttttcactttgtatccttaacttgagcatcaccaagaaaagaattccacagaaatgctggaaaatcatgcacacgtgttcttcagcctgtgtaacgcccctgcaagcacagggtaaacaggtctcatcttcatggcacttcccagttatgtggggccagcattctgtctggaatgttcggaaactggaatgttcggtagtgtgtcctgcagaattcatcatttcggcaagtgtgaattacactgaggcaagtggggggtagaactatattcagtgcacatagcttgctgtgaagagtttctttggattgctgacagggtatatcagattgagcacagtcacaaaaagccaacatctgaaaggtaaagggccaactgtgcattacaaaccacatcccctacacacgcctctgtcacctccaaaattatctgtcttattggtgcacttttttccaaaaagtttgtttactgtacagtggaggtcaaaatgtgcacaatcatttgtttgggaagaggattcattttctgagcttaacgtaacagccaggaaaggcgcgccagagggcagaggaagtcttcta

SEQ ID NO: 23 - DNA, 63 bp, Thosea asigna virus
  source 1..63: mol_type = other DNA
ggaagcggagagggcacaggaagtctgctaacatgcggtgacgtcgaggagaatcctggacct

SEQ ID NO: 24 - protein, 21 aa, Thosea asigna virus
GSGEGRGSLLTCGDVEENPGP

SEQ ID NO: 25 - DNA, 69 bp, Equine rhinitis A virus
  source 1..69: mol_type = genomic DNA
ggaagcggacagtgtactaattatgctctcttgaaattggctggagatgttgagagcaaccctggacct

SEQ ID NO: 26 - protein, 23 aa, Equine rhinitis A virus
GSGQCTNYALLKLAGDVESNPGP

SEQ ID NO: 27 - DNA, 75 bp, Foot-and-mouth disease virus
  source 1..75: mol_type = other DNA
ggaagcggagtgaaacagactttgaattttgaccttctcaagttggcgggagacgtggagtccaaccctggacct

SEQ ID NO: 28 - protein, 25 aa, Foot-and-mouth disease virus
GSGVKQTLNFDLLKLAGDVESNPGP